SEQUENCE LISTING (61 sequences)

SEQ ID NO: 1 - DNA, 3393 bp, Homo sapiens
attcgggggcgcgagctgccccagtaaatggaaatgttttctaacatataaaaacctacagaagaagaaaataattttctggatcaaattagaagtctgtattatattgatgtctccagattcaaatatattagaaagcagccgtggagacaaccatcttcattttgggagaaataactaaagcccgcctcaagcattagaactacagacaaaccctgatgcgacctctccagattgtcccaagtcgattgatttcccagctatattgtggcctgaagcctccagcgtccacacgaaaccagatttgcctgaaaatggctcggccaagttcaagtatggcagattttcgaaagttttttgcaaaagcaaagcacatagtcatcatctcaggagctggtgttagtgcagaaagtggtgttccgaccttcagaggagctggaggttattggagaaaatggcaagcccaggacctggcgactcccctggcctttgcccacaacccgtcccgggtgtgggagttctaccactaccggcgggaggtcatggggagcaaggagcccaacgccgggcaccgcgccatagccgagtgtgagacccggctgggcaagcagggccggcgagtcgtggtcatcacccagaacatcgatgagctgcaccgcaaggctggcaccaagaaccttctggagatccatggtagcttatttaaaactcgatgtacctcttgtggagttgtggctgagaattacaagagtccaatttgtccagctttatcaggaaaaggtgctccagaacctggaactcaagatgccagcatcccagttgagaaacttccccggtgtgaagaggcaggctgcgggggcttgctgcgacctcacgtcgtgtggtttggagaaaacctggatcctgccattctggaggaggttgacagagagctcgcccactgtgatttatgtctagtggtgggcacttcctctgtggtgtacccagcagccatgtttgccccccaggtggctgccaggggcgtgccagtggctgaatttaacacggagaccaccccagctacgaacagattcaggtttcatttccagggaccctgtggaacgactcttcctgaagcccttgcctgtcatgaaaatgaaactgtttcttaagtgtcctggggaagaaagaaattacagtatatctaagaactaggccacacgcagaggagaaatggtcttatgggtggtgagctgagtactgaacaatctaaaaatagcctctgattccctcgctggaatccaacctgttgataagtgatgggggtttagaagtagcaaagagcacccacattcaaaagtcacagaactggaaagttaattcatattatttggtttgaactgaaacgtgaggtatctttgatgtgtatggttggttattgggagggaaaaattttgtaaattagattgtctaaaaaaaatagttattctgattatatttttgttatctgggcaaagtagaagtcaaggggtaaaaaccctactattctgatttttgcacaagttttagtggaaaataaaatcacactctacagtaggtaatttattgtataaagacattaccccacgatatggctttattagggactttttttttttttttttgagacagagtttcactcttgttgcccaggctggagtgcagtggtgcgatctcagctcacagcaacctccgcctcccgggttcaagagattctcctgcctcagcctcatgagtagctgggattacaggtatgtaccaccacacccagctaattttgtatttttagtagagacggggtttctccatgttggtcaggctggtcttaaactctcgacctcaggtgatctgcccgcctcggcctcccaaagtgctgagattacgggcatgagccaccgcacccggcttactgggggctttttaaccttgtttggctacattacctcagtgaacaaggggaagctcacaacaggaactcacacaaagaaggaagagaacagtaccaacaaggtagaacatcacgatgagaagaaagaatgatgcaaatatgtggacccccaggataaacacagctgcccaaaatttagctctggcccccttagatgggggaagtcaacagtgaagtgtttctcagcatcataaaggcagaagctgtggttcctgtggggcctgccaaccgttcccagatgctgaactctgctgggagttttttccatgggactttaaaaaatgatgcccttaggttgggccagacctctgttaacttcagtagggatggcaccaggttcaagaggccaaagaagagacctggagctagtgaaggaaacatagggtttatttggggaaccttacagggtggtccagtggccgcgggctggacagaactgcaaccacttataaaaagcatgcagtttacatagcactttcactcagcaccctcccctcagcagcctccacgtggcaaccctcacttcttaagttattgctgtcagatgcatctgccatacagggtcattctcaggggatgcttaagttatttctgtcaggtacatcttccatacactttactaccttggagtaaagtagtaagaatacagctttttccttaacctttaccagctaactcagtgcttaggggccttggaatgcctgctgtccagcaggtgtcacaggcctgactgggaggcatggccattatcaacagtgtatgaaggtcacatatggctccctgaagtgattacatacttggaccacatcacctcagccccttgcaaaattgcatttaatgttacacccttggcttttgtagaaacaggcaacaagacactatcatataaaactttgtactgcattgcaaggcataacctttaataaatcccagtggtcctttgtgtagggaacggggatgctcatagcctatggggcggctggagaaccagtcagggaccctgagggctcgatgtacactttacatgggtgggccaaggagtttacactgagggcactggtaatacctgtaatagtcttatagtacttataaagcagttttgcacataaaataccatcatgcacttataagtttgttcttgggctgctccaagttaactttattcattttcctagtgttagtgtctcagggagtctgattatatttttgatttgtaatttctatctgactaaggcctagagatttcaaaactgttctttgcaattcctctcatactgaacctctggttcagcagctttttttgttgttgttgttttcaagttttatcatttttgttcctatttggttttgtcgtttttaaattgagaattgcttctaaaacagaagacatgaaaagagaattaaaaatacaatatatgtgtaagata

SEQ ID NO: 2 - protein, 310 aa, Homo sapiens
MRPLQIVPSRLISQLYCGLKPPASTRNQICLKMARPSSSMADFRKFFAKAKHIVIISGAGVSAESGVPTFRGAGGYWRKWQAQDLATPLAFAHNPSRVWEFYHYRREVMGSKEPNAGHRAIAECETRLGKQGRRVVVITQNIDELHRKAGTKNLLEIHGSLFKTRCTSCGVVAENYKSPICPALSGKGAPEPGTQDASIPVEKLPRCEEAGCGGLLRPHVVWFGENLDPAILEEVDRELAHCDLCLVVGTSSVVYPAAMFAPQVAARGVPVAEFNTETTPATNRFRFHFQGPCGTTLPEALACHENETVS

SEQ ID NO: 3 - DNA, 930 bp, Artificial Sequence
  Recombinant nucleic acid
atgcgacctctccagattgtcccaagtcgattgatttcccagctatattgtggcctgaagcctccagcgtccacacgaaaccagatttgcctgaaaatggctcggccaagttcaagtatggcagattttcgaaagttttttgcaaaagcaaagcacatagtcatcatctcaggagctggtgttagtgcagaaagtggtgttccgaccttcagaggagctggaggttattggagaagatggcaagcccaggacctggcgactcccctggcctttgcccacaacccgtcccgggtgtgggagttctaccactaccggcgggaggtcatggggagcagggagcccaacgccgggcaccgcgccatagccgagtgtgagacccggctgggcaagcagggccggcgagtcgtggtcatcacccagaacatcgatgagctgcatcgcagggctggcaccaggaaccttctggagatccatggtagcttatttaaaactcgatgtacctcttgtggagttgtggctgagaattacaagagtccaatttgtccagctttatcaggaaaaggtgctccagaacctggaactcaagatgccagcatcccagttgagaaacttccccggtgtgaagaggcaggctgcgggggcttgctgcgacctcacgtcgtgtggtttggagaaaacctggatcctgccattctggaggaggttgacagagagctcgcccactgtgatttatgtctagtggtgggcacttcctctgtggtgtacccagcagccatgtttgccccccaggtggctgccaggggcgtgccagtggctgaatttaacacggagaccaccccagctacgaacagattcaggtttcatttccagggaccctgtggaacgactcttcctgaagcccttgcctgtcatgaaaatgaaactgtttct

SEQ ID NO: 4 - protein, 310 aa, Artificial Sequence
  Recombinant protein
MRPLQIVPSRLISQLYCGLKPPASTRNQICLKMARPSSSMADFRKFFAKAKHIVIISGAGVSAESGVPTFRGAGGYWRRWQAQDLATPLAFAHNPSRVWEFYHYRREVMGSREPNAGHRAIAECETRLGKQGRRVVVITQNIDELHRRAGTRNLLEIHGSLFKTRCTSCGVVAENYKSPICPALSGKGAPEPGTQDASIPVEKLPRCEEAGCGGLLRPHVVWFGENLDPAILEEVDRELAHCDLCLVVGTSSVVYPAAMFAPQVAARGVPVAEFNTETTPATNRFRFHFQGPCGTTLPEALACHENETVS

SEQ ID NO: 5 - DNA, 4503 bp, Homo sapiens
atgacgaggattttgacagctttcaaagtggtgaggacactgaagactggttttggctttaccaatgtgactgcacaccaaaaatggaaattttcaagacctggcatcaggctcctttctgtcaaggcacagacagcacacattgtcctggaagatggaactaagatgaaaggttactcctttggccatccatcctctgttgctggtgaagtggtttttaatactggcctgggagggtacccagaagctattactgaccctgcctacaaaggacagattctcacaatggccaaccctattattgggaatggtggagctcctgatactactgctctggatgaactgggacttagcaaatatttggagtctaatggaatcaaggtttcaggtttgctggtgctggattatagtaaagactacaaccactggctggctaccaagagtttagggcaatggctacaggaagaaaaggttcctgcaatttatggagtggacacaagaatgctgactaaaataattcgggataagggtaccatgcttgggaagattgaatttgaaggtcagcctgtggattttgtggatccaaataaacagaatttgattgctgaggtttcaaccaaggatgtcaaagtgtacggcaaaggaaaccccacaaaagtggtagctgtagactgtgggattaaaaacaatgtaatccgcctgctagtaaagcgaggagctgaagtgcacttagttccctggaaccatgatttcaccaagatggagtatgatgggattttgatcgcgggaggaccggggaacccagctcttgcagaaccactaattcagaatgtcagaaagattttggagagtgatcgcaaggagccattgtttggaatcagtacaggaaacttaataacaggattggctgctggtgccaaaacctacaagatgtccatggccaacagagggcagaatcagcctgttttgaatatcacaaacaaacaggctttcattactgctcagaatcatggctatgccttggacaacaccctccctgctggctggaaaccactttttgtgaatgtcaacgatcaaacaaatgaggggattatgcatgagagcaaacccttcttcgctgtgcagttccacccagaggtcaccccggggccaatagacactgagtacctgtttgattcctttttctcactgataaagaaaggaaaagctaccaccattacatcagtcttaccgaagccagcactagttgcatctcgggttgaggtttccaaagtccttattctaggatcaggaggtctgtccattggtcaggctggagaatttgattactcaggatctcaagctgtaaaagccatgaaggaagaaaatgtcaaaactgttctgatgaacccaaacattgcatcagtccagaccaatgaggtgggcttaaagcaagcggatactgtctactttcttcccatcacccctcagtttgtcacagaggtcatcaaggcagaacagccagatgggttaattctgggcatgggtggccagacagctctgaactgtggagtggaactattcaagagaggtgtgctcaaggaatatggtgtgaaagtcctgggaacttcagttgagtccattatggctacggaagacaggcagctgttttcagataaactaaatgagatcaatgaaaagattgctccaagttttgcagtggaatcgattgaggatgcactgaaggcagcagacaccattggctacccagtgatgatccgttccgcctatgcactgggtgggttaggctcaggcatctgtcccaacagagagactttgatggacctcagcacaaaggcctttgctatgaccaaccaaattctggtggagaagtcagtgacaggttggaaagaaatagaatatgaagtggttcgagatgctgatgacaattgtgtcactgtctgtaacatggaaaatgttgatgccatgggtgttcacacaggtgactcagttgttgtggctcctgcccagacactctccaatgccgagtttcagatgttgagacgtacttcaatcaatgttgttcgccacttgggcattgtgggtgaatgcaacattcagtttgcccttcatcctacctcaatggaatactgcatcattgaagtgaatgccagactgtcccgaagctctgctctggcctcaaaagccactggctacccattggcattcattgctgcaaagattgccctaggaatcccacttccagaaattaagaacgtcgtatccgggaagacatcagcctgttttgaacctagcctggattacatggtcaccaagattccccgctgggatcttgaccgttttcatggaacatctagccgaattggtagctctatgaaaagtgtaggagaggtcatggctattggtcgtacctttgaggagagtttccagaaagctttacggatgtgccacccatctatagaaggtttcactccccgtctcccaatgaacaaagaatggccatctaatttagatcttagaaaagagttgtctgaaccaagcagcacgcgtatctatgccattgccaaggccattgatgacaacatgtcccttgatgagattgagaagctcacatacattgacaagtggtttttgtataagatgcgtgatattttaaacatggaaaagacactgaaaggcctcaacagtgagtccatgacagaagaaaccctgaaaagggcaaaggagattgggttctcagataagcagatttcaaaatgccttgggctcactgaggcccagacaagggagctgaggttaaagaaaaacatccacccttgggttaaacagattgatacactggctgcagaatacccatcagtaacaaactatctctatgttacctacaatggtcaggagcatgatgtcaattttgatgaccatggaatgatggtgctaggctgtggtccatatcacattggcagcagtgtggaatttgattggtgtgctgtctctagtatccgcacactgcgtcaacttggcaagaagacggtggtggtgaattgcaatcctgagactgtgagcacagactttgatgagtgtgacaaactgtactttgaagagttgtccttggagagaatcctagacatctaccatcaggaggcatgtggtggctgcatcatatcagttggaggccagattccaaacaacctggcagttcctctatacaagaatggtgtcaagatcatgggcacaagccccctgcagatcgacagggctgaggatcgctccatcttctcagctgtcttggatgagctgaaggtggctcaggcaccttggaaagctgttaatactttgaatgaagcactggaatttgcaaagtctgtggactacccctgcttgttgaggccttcctatgttttgagtgggtctgctatgaatgtggtattctctgaggatgagatgaaaaaattcctagaagaggcgactagagtttctcaggagcacccagtggtgctgacaaaatttgttgaaggggcccgagaagtagaaatggacgctgttggcaaagatggaagggttatctctcatgccatctctgaacatgttgaagatgcaggtgtccactcgggagatgccactctgatgctgcccacacaaaccatcagccaaggggccattgaaaaggtgaaggatgctacccggaagattgcaaaggcttttgccatctctggtccattcaacgtccaatttcttgtcaaaggaaatgatgtcttggtgattgagtgtaacttgagagcttctcgatccttcccctttgtttccaagactcttggggttgacttcattgatgtggccaccaaggtgatgattggagagaatgttgatgagaaacatcttccaacattggaccatcccataattcctgctgactatgttgcaattaaggctcccatgttttcctggccccggttgagggatgctgaccccattctgagatgtgagatggcttccactggagaggtggcttgctttggtgaaggtattcatacagccttcctaaaggcaatgctttccacaggatttaagataccccagaaaggcatcctgataggcatccagcaatcattccggccaagattccttggtgtggctgaacaattacacaatgaaggtttcaagctgtttgccacggaagccacatcagactggctcaacgccaacaatgtccctgccaccccagtggcatggccgtctcaagaaggacagaatcccagcctctcttccatcagaaaattgattagagatggcagcattgacctagtgattaaccttcccaacaacaacactaaatttgtccatgataattatgtgattcggaggacagctgttgatagtggaatccctctcctcactaattttcaggtgaccaaactttttgctgaagctgtgcagaaatctcgcaaggtggactccaagagtcttttccactacaggcagtacagtgctggaaaagcagcatag

SEQ ID NO: 6 - protein, 1500 aa, Homo sapiens
MTRILTAFKVVRTLKTGFGFTNVTAHQKWKFSRPGIRLLSVKAQTAHIVLEDGTKMKGYSFGHPSSVAGEVVFNTGLGGYPEAITDPAYKGQILTMANPIIGNGGAPDTTALDELGLSKYLESNGIKVSGLLVLDYSKDYNHWLATKSLGQWLQEEKVPAIYGVDTRMLTKIIRDKGTMLGKIEFEGQPVDFVDPNKQNLIAEVSTKDVKVYGKGNPTKVVAVDCGIKNNVIRLLVKRGAEVHLVPWNHDFTKMEYDGILIAGGPGNPALAEPLIQNVRKILESDRKEPLFGISTGNLITGLAAGAKTYKMSMANRGQNQPVLNITNKQAFITAQNHGYALDNTLPAGWKPLFVNVNDQTNEGIMHESKPFFAVQFHPEVTPGPIDTEYLFDSFFSLIKKGKATTITSVLPKPALVASRVEVSKVLILGSGGLSIGQAGEFDYSGSQAVKAMKEENVKTVLMNPNIASVQTNEVGLKQADTVYFLPITPQFVTEVIKAEQPDGLILGMGGQTALNCGVELFKRGVLKEYGVKVLGTSVESIMATEDRQLFSDKLNEINEKIAPSFAVESIEDALKAADTIGYPVMIRSAYALGGLGSGICPNRETLMDLSTKAFAMTNQILVEKSVTGWKEIEYEVVRDADDNCVTVCNMENVDAMGVHTGDSVVVAPAQTLSNAEFQMLRRTSINVVRHLGIVGECNIQFALHPTSMEYCIIEVNARLSRSSALASKATGYPLAFIAAKIALGIPLPEIKNVVSGKTSACFEPSLDYMVTKIPRWDLDRFHGTSSRIGSSMKSVGEVMAIGRTFEESFQKALRMCHPSIEGFTPRLPMNKEWPSNLDLRKELSEPSSTRIYAIAKAIDDNMSLDEIEKLTYIDKWFLYKMRDILNMEKTLKGLNSESMTEETLKRAKEIGFSDKQISKCLGLTEAQTRELRLKKNIHPWVKQIDTLAAEYPSVTNYLYVTYNGQEHDVNFDDHGMMVLGCGPYHIGSSVEFDWCAVSSIRTLRQLGKKTVVVNCNPETVSTDFDECDKLYFEELSLERILDIYHQEACGGCIISVGGQIPNNLAVPLYKNGVKIMGTSPLQIDRAEDRSIFSAVLDELKVAQAPWKAVNTLNEALEFAKSVDYPCLLRPSYVLSGSAMNVVFSEDEMKKFLEEATRVSQEHPVVLTKFVEGAREVEMDAVGKDGRVISHAISEHVEDAGVHSGDATLMLPTQTISQGAIEKVKDATRKIAKAFAISGPFNVQFLVKGNDVLVIECNLRASRSFPFVSKTLGVDFIDVATKVMIGENVDEKHLPTLDHPIIPADYVAIKAPMFSWPRLRDADPILRCEMASTGEVACFGEGIHTAFLKAMLSTGFKIPQKGILIGIQQSFRPRFLGVAEQLHNEGFKLFATEATSDWLNANNVPATPVAWPSQEGQNPSLSSIRKLIRDGSIDLVINLPNNNTKFVHDNYVIRRTAVDSGIPLLTNFQVTKLFAEAVQKSRKVDSKSLFHYRQYSAGKAA

SEQ ID NO: 7 - DNA, 969 bp, Artificial Sequence
  Recombinant nucleic acid
atgcgacctctccagattgtcccaagtcgattgatttcccagctatattgtggcctgaagcctccagcgtccacacgaaaccagatttgcctgaaaatggctcggccaagttcaagtatggcagattttcgaaagttttttgcaaaagcaaagcacatagtcatcatctcaggagctggtgttagtgcagaaagtggtgttccgaccttcagaggagctggaggttattggagaaaatggcaagcccaggacctggcgactcccctggcctttgcccacaacccgtcccgggtgtgggagttctaccactaccggcgggaggtcatggggagcaaggagcccaacgccgggcaccgcgccatagccgagtgtgagacccggctgggcaagcagggccggcgagtcgtggtcatcacccagaacatcgatgagctgcaccgcaaggctggcaccaagaaccttctggagatccatggtagcttatttaaaactcgatgtacctcttgtggagttgtggctgagaattacaagagtccaatttgtccagctttatcaggaaaaggtgctccagaacctggaactcaagatgccagcatcccagttgagaaacttccccggtgtgaagaggcaggctgcgggggcttgctgcgacctcacgtcgtgtggtttggagaaaacctggatcctgccattctggaggaggttgacagagagctcgcccactgtgatttatgtctagtggtgggcacttcctctgtggtgtacccagcagccatgtttgccccccaggtggctgccaggggcgtgccagtggctgaatttaacacggagaccaccccagctacgaacagattcaggtttcatttccagggaccctgtggaacgactcttcctgaagcccttgcctgtcatgaaaatgaaactgtttctgaattcggaggagactacaaggacgacgatgacaagtaa

SEQ ID NO: 8 - protein, 322 aa, Artificial Sequence
  Recombinant protein
MRPLQIVPSRLISQLYCGLKPPASTRNQICLKMARPSSSMADFRKFFAKAKHIVIISGAGVSAESGVPTFRGAGGYWRKWQAQDLATPLAFAHNPSRVWEFYHYRREVMGSKEPNAGHRAIAECETRLGKQGRRVVVITQNIDELHRKAGTKNLLEIHGSLFKTRCTSCGVVAENYKSPICPALSGKGAPEPGTQDASIPVEKLPRCEEAGCGGLLRPHVVWFGENLDPAILEEVDRELAHCDLCLVVGTSSVVYPAAMFAPQVAARGVPVAEFNTETTPATNRFRFHFQGPCGTTLPEALACHENETVSEFGGDYKDDDDK

SEQ ID NO: 9 - DNA, 969 bp, Artificial Sequence
  Recombinant nucleic acid
atgcgacctctccagattgtcccaagtcgattgatttcccagctatattgtggcctgaagcctccagcgtccacacgaaaccagatttgcctgaaaatggctcggccaagttcaagtatggcagattttcgaaagttttttgcaaaagcaaagcacatagtcatcatctcaggagctggtgttagtgcagaaagtggtgttccgaccttcagaggagctggaggttattggagaagatggcaagcccaggacctggcgactcccctggcctttgcccacaacccgtcccgggtgtgggagttctaccactaccggcgggaggtcatggggagcagggagcccaacgccgggcaccgcgccatagccgagtgtgagacccggctgggcaagcagggccggcgagtcgtggtcatcacccagaacatcgatgagctgcatcgcagggctggcaccaggaaccttctggagatccatggtagcttatttaaaactcgatgtacctcttgtggagttgtggctgagaattacaagagtccaatttgtccagctttatcaggaaaaggtgctccagaacctggaactcaagatgccagcatcccagttgagaaacttccccggtgtgaagaggcaggctgcgggggcttgctgcgacctcacgtcgtgtggtttggagaaaacctggatcctgccattctggaggaggttgacagagagctcgcccactgtgatttatgtctagtggtgggcacttcctctgtggtgtacccagcagccatgtttgccccccaggtggctgccaggggcgtgccagtggctgaatttaacacggagaccaccccagctacgaacagattcaggtttcatttccagggaccctgtggaacgactcttcctgaagcccttgcctgtcatgaaaatgaaactgtttctgaattcggaggagactacaaggacgacgatgacaagtaa

SEQ ID NO: 10 - protein, 322 aa, Artificial Sequence
  Recombinant protein
MRPLQIVPSRLISQLYCGLKPPASTRNQICLKMARPSSSMADFRKFFAKAKHIVIISGAGVSAESGVPTFRGAGGYWRRWQAQDLATPLAFAHNPSRVWEFYHYRREVMGSREPNAGHRAIAECETRLGKQGRRVVVITQNIDELHRRAGTRNLLEIHGSLFKTRCTSCGVVAENYKSPICPALSGKGAPEPGTQDASIPVEKLPRCEEAGCGGLLRPHVVWFGENLDPAILEEVDRELAHCDLCLVVGTSSVVYPAAMFAPQVAARGVPVAEFNTETTPATNRFRFHFQGPCGTTLPEALACHENETVSEFGGDYKDDDDK

SEQ ID NO: 11 - DNA, 5186 bp, Artificial Sequence
  Recombinant nucleic acid
ttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttggggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatcaagtttgtacaaaaaagcaggctgccaccatgcgacctctccagattgtcccaagtcgattgatttcccagctatattgtggcctgaagcctccagcgtccacacgaaaccagatttgcctgaaaatggctcggccaagttcaagtatggcagattttcgaaagttttttgcaaaagcaaagcacatagtcatcatctcaggagctggtgttagtgcagaaagtggtgttccgaccttcagaggagctggaggttattggagaagatggcaagcccaggacctggcgactcccctggcctttgcccacaacccgtcccgggtgtgggagttctaccactaccggcgggaggtcatggggagcagggagcccaacgccgggcaccgcgccatagccgagtgtgagacccggctgggcaagcagggccggcgagtcgtggtcatcacccagaacatcgatgagctgcatcgcagggctggcaccaggaaccttctggagatccatggtagcttatttaaaactcgatgtacctcttgtggagttgtggctgagaattacaagagtccaatttgtccagctttatcaggaaaaggtgctccagaacctggaactcaagatgccagcatcccagttgagaaacttccccggtgtgaagaggcaggctgcgggggcttgctgcgacctcacgtcgtgtggtttggagaaaacctggatcctgccattctggaggaggttgacagagagctcgcccactgtgatttatgtctagtggtgggcacttcctctgtggtgtacccagcagccatgtttgccccccaggtggctgccaggggcgtgccagtggctgaatttaacacggagaccaccccagctacgaacagattcaggtttcatttccagggaccctgtggaacgactcttcctgaagcccttgcctgtcatgaaaatgaaactgtttctgaattcggaggagactacaaggacgacgatgacaagtaaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgac

SEQ ID NO: 12 - DNA, 6122 bp, Artificial Sequence
  Recombinant nucleic acid
tggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgatatcgccgccaccatgcgacctctccagattgtcccaagtcgattgatttcccagctatattgtggcctgaagcctccagcgtccacacgaaaccagatttgcctgaaaatggctcggccaagttcaagtatggcagattttcgaaagttttttgcaaaagcaaagcacatagtcatcatctcaggagctggtgttagtgcagaaagtggtgttccgaccttcagaggagctggaggttattggagaagatggcaagcccaggacctggcgactcccctggcctttgcccacaacccgtcccgggtgtgggagttctaccactaccggcgggaggtcatggggagcagggagcccaacgccgggcaccgcgccatagccgagtgtgagacccggctgggcaagcagggccggcgagtcgtggtcatcacccagaacatcgatgagctgcatcgcagggctggcaccaggaaccttctggagatccatggtagcttatttaaaactcgatgtacctcttgtggagttgtggctgagaattacaagagtccaatttgtccagctttatcaggaaaaggtgctccagaacctggaactcaagatgccagcatcccagttgagaaacttccccggtgtgaagaggcaggctgcgggggcttgctgcgacctcacgtcgtgtggtttggagaaaacctggatcctgccattctggaggaggttgacagagagctcgcccactgtgatttatgtctagtggtgggcacttcctctgtggtgtacccagcagccatgtttgccccccaggtggctgccaggggcgtgccagtggctgaatttaacacggagaccaccccagctacgaacagattcaggtttcatttccagggaccctgtggaacgactcttcctgaagcccttgcctgtcatgaaaatgaaactgtttctgaattcggaggagactacaaggacgacgatgacaagtaaaagcttctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagc

SEQ ID NO: 13 - DNA, 23 bp, Artificial Sequence
  Primer
gttattggagaagatggcaagcc

SEQ ID NO: 14 - DNA, 23 bp, Artificial Sequence
  Primer
ggcttgccatcttctccaataac

SEQ ID NO: 15 - DNA, 23 bp, Artificial Sequence
  Primer
catggggagcagggagcccaacg

SEQ ID NO: 16 - DNA, 23 bp, Artificial Sequence
  Primer
cgttgggctccctgctccccatg

SEQ ID NO: 17 - DNA, 33 bp, Artificial Sequence
  Primer
ctgcaccgcagggctggcaccaggaaccttctg

SEQ ID NO: 18 - DNA, 33 bp, Artificial Sequence
  Primer
cagaaggttcctggtgccagccctgcggtgcag

SEQ ID NO: 19 - protein, 8 aa, Artificial Sequence
  Synthetic peptide
DYKDDDDK

SEQ ID NO: 20 - protein, 9 aa, Artificial Sequence
  Synthetic peptide
NANNPDWDF

SEQ ID NO: 21 - protein, 11 aa, Artificial Sequence
  Primer
CEQKLISEEDL

SEQ ID NO: 22 - protein, 11 aa, Artificial Sequence
  Synthetic peptide
YGRKKRRQRRR

SEQ ID NO: 23 - protein, 18 aa, Artificial Sequence
  Synthetic peptide
RGGRLSYSRRRFSTSTGR

SEQ ID NO: 24 - protein, 10 aa, Artificial Sequence
  Synthetic peptide
RRLSYSRRRF

SEQ ID NO: 25 - protein, 12 aa, Artificial Sequence
  Synthetic peptide
PIRRRKKLRRLK

SEQ ID NO: 26 - protein, 12 aa, Artificial Sequence
  Synthetic peptide
RRQRRTSKLMKR

SEQ ID NO: 27 - protein, 15 aa, Artificial Sequence
  Synthetic peptide
RRRRNRTRRNRRRVR

SEQ ID NO: 28 - protein, 19 aa, Artificial Sequence
  Synthetic peptide
KMTRAQRRAAARRNRWTAR

SEQ ID NO: 29 - protein, 13 aa, Artificial Sequence
  Synthetic peptide
TRRQRTRRARRNR

SEQ ID NO: 30 - protein, 13 aa, Artificial Sequence
  Synthetic peptide
GRKKRRQRRRPPQ

SEQ ID NO: 31 - protein, 13 aa, Artificial Sequence
  Synthetic peptide
GRRRRRRRRRPPQ

SEQ ID NO: 32 - protein, 15 aa, Artificial Sequence
  Synthetic peptide
RQIKWFQNRRMKWKK

SEQ ID NO: 33 - protein, 17 aa, Artificial Sequence
  Synthetic peptide
KLALKLALKLALALKLA

SEQ ID NO: 34 - protein, 27 aa, Artificial Sequence
  Synthetic peptide
MGLGLHLLVLAAALQGAWSQPKKKRKV

SEQ ID NO: 35 - protein, 27 aa, Artificial Sequence
  Synthetic peptide
GALFLGWLGAAGSTMGAWSQPKKKRKV

SEQ ID NO: 36 - protein, 27 aa, Artificial Sequence
  Synthetic peptide
GALFLGFLGAAGSTMGAWSQPKKKRKV

SEQ ID NO: 37 - protein, 27 aa, Artificial Sequence
  Synthetic peptide
GALFLGFLGAAGSTMGAWSQPKSKRKV

SEQ ID NO: 38 - protein, 21 aa, Artificial Sequence
  Synthetic peptide
KETWFETWFTEWSQPKKKRKV

SEQ ID NO: 39 - protein, 27 aa, Artificial Sequence
  Synthetic peptide
GWTLNSAGYLLGKINLKALAALAKKIL

SEQ ID NO: 40 - protein, 21 aa, Artificial Sequence
  Synthetic peptide
KETWWETWWTEWSQPKKKRKV

SEQ ID NO: 41 - protein, 6 aa, Artificial Sequence
  Synthetic peptide
PFVYLI

SEQ ID NO: 42 - protein, 19 aa, Artificial Sequence
  Synthetic peptide
KKAKNKQLGHEEDYALGKD

SEQ ID NO: 43 - protein, 9 aa, Artificial Sequence
  Synthetic peptide
KKKEKEVKK

SEQ ID NO: 44 - protein, 15 aa, Artificial Sequence
  Synthetic peptide
KTAHIVLEDGTKMKG

SEQ ID NO: 45 - protein, 25 aa, Artificial Sequence
  Synthetic peptide
KISLPHPMEIGENLDGTLKSRKRRK

SEQ ID NO: 46 - protein, 15 aa, Artificial Sequence
  Synthetic peptide
KKKNDFEQGELYLKE

SEQ ID NO: 47 - protein, 15 aa, Artificial Sequence
  Synthetic peptide
KDKYKQIFLGGVDKR

SEQ ID NO: 48 - protein, 22 aa, Artificial Sequence
  Synthetic peptide
KGKKLVKKKIGKKDAGKKEGKC

SEQ ID NO: 49 - protein, 17 aa, Artificial Sequence
  Synthetic peptide
KKNSEGLLKNKEKNQKL

SEQ ID NO: 50 - protein, 13 aa, Artificial Sequence
  Synthetic peptide
KDAYIKKQNLEKA

SEQ ID NO: 51 - protein, 16 aa, Artificial Sequence
  Synthetic peptide
KAFKNKETLIIEPEKN

SEQ ID NO: 52 - protein, 13 aa, Artificial Sequence
  Synthetic peptide
KDVEKKLNKVTKF

SEQ ID NO: 53 - protein, 16 aa, Artificial Sequence
  Synthetic peptide
KELGEKISQLKDELKT

SEQ ID NO: 54 - protein, 12 aa, Artificial Sequence
  Synthetic peptide
KKIVAENHLKKI

SEQ ID NO: 55 - protein, 14 aa, Artificial Sequence
  Synthetic peptide
RKKVETEAKIKQKL

SEQ ID NO: 56 - protein, 28 aa, Artificial Sequence
  Synthetic peptide
KKETKGPAAENLEAKPVQAPTVKKAEKD

SEQ ID NO: 57 - protein, 24 aa, Artificial Sequence
  Synthetic peptide
KKFGGQDIFMTEEQKKYYNAMKKL

SEQ ID NO: 58 - protein, 33 aa, Artificial Sequence
  Synthetic peptide
KKDTQTKSIISETSNKIDTEIASLKTLMESSKL

SEQ ID NO: 59 - protein, 34 aa, Artificial Sequence
  Synthetic peptide
KLGKMDRVVLGWTAVFWLTAMVEGLQVTVPDKKK

SEQ ID NO: 60 - protein, 15 aa, Artificial Sequence
  Synthetic peptide
KYKIKTIQDLVSLKE

SEQ ID NO: 61 - protein, 14 aa, Artificial Sequence
  Synthetic peptide
KRKMRKGQHLDLKA

(19) United States
(12) Patent Application Publication
(10) Pub. No.: US 2023/0181672 A1
(43) Pub. Date: Jun. 15, 2023

(54) ABERRANT POST-TRANSLATIONAL MODIFICATIONS (PTMS) IN METHYL- AND PROPIONIC ACIDEMIA AND A MUTANT SIRTUIN (SIRT) TO METABOLIZE PTMS

(71) Applicant: The U.S.A., as represented by the Secretary, Department of Health and Human Services, Bethesda, MD (US)

(72) Inventors: Charles P. Venditti, Potomac, MD (US); PamelaSara Elbaz Head, Rockville, MD (US)

(73) Assignee: The U.S.A., as represented by the Secretary, Department of Health and Human Services, Bethesda, MD (US)

(21) Appl. No.: 17/923,394

(22) PCT Filed: Apr. 20, 2021
(86) PCT No.: PCT/US2021/028228

§ 371 Date: Nov. 4, 2022

Related U.S. Application Data
(60) Provisional application No. 63/021,179, filed on May 7, 2020.

(51) Int. Cl. A61K 38/00; C07K 14/765

(52) U.S. Cl. CPC A61K 38/00; C07K 14/765; C07K 2319/43; C12N 2750/14143

(57) ABSTRACT

This application provides the first observation of methylmalonylation/malonylation in organic acidemias (OAs), such as methylmalonic acidemia (MMA) and propionic acidemia (PA), which results in modification of enzymes in key pathways dysregulated in OAs, including sirtuin 5 (SIRT5). Hyperacylation of SIRT5 prevents it from de-acylating CPS1 (including removing methymalonyllation), which prevents activation of CPS1 and likewise, inhibits a key component of the glycine cleavage system, GCSH. Based on these observations, provided herein is a mutant form of SIRT5 containing four mutated lysines that cannot accept acyl groups, methods of its use for treating OA patients, and kits.

### CROSS REFERENCE TO RELATED APPLICATIONS

This application claims the benefit of U.S. Provisional Application No. 63/021,179, filed May 7, 2020, which is herein incorporated by reference in its entirety.

### STATEMENT OF GOVERNMENT INTEREST

The instant application was made with government support from National Human Genome Research Institute (NHGRI), which is part of the National Institutes of Health (NIH) under project numbers 1 ZIAHG 200318-15. The United States government has certain rights in this invention.

### FIELD

Provided herein is a mutant form of sirtuin 5 (SIRTS) containing four mutated lysines that cannot accept acyl groups on catalytic lysines, and methods of its use for treating organic acidemia (OA) patients.

### BACKGROUND

Organic acidemias (OAs), such as methylmalonic acidemia (MMA) and propionic acidemia (PA), are a group of inborn errors of metabolism that typically arise from defects in the catabolism of amino, organic and fatty acids. OAs are difficult to treat and have multisystemic manifestations, leading to increased morbidity and mortality. Methods for improved treatments and diagnosis of OAs are needed.

### SUMMARY

The inventors have unexpectedly discovered a new pathophysiological consequence of impaired acyl-CoA metabolism in OAs: the accumulation of aberrant posttranslational modifications (PTMs) that modify enzymes in critical intracellular pathways, especially during periods of increased stress. In addition, a new PTM, methylmalonylation, was identified in samples from mice and humans with methylmalonic acidemia (MMA). The accumulation of methylmalonylation, malonylation, and propionylation acylation PTMs on proteins in critical metabolic pathways may explain the diverse, and severe, effects observed in patients with MMA.

The inventors have also found that in addition to known demalonylation activity, the deacylase enzyme SIRTS also exhibits demethylmalonylation and depropionylation activity, which should allow for reversal of these aberrant PTMs in OAs. However, hyperacylation of SIRTS itself inhibits its enzymatic activity, preventing it from deacylating lysine residues on key metabolic targets including carbamoylphosphate synthase 1 (CPS1) and glycine cleavage system H protein (GCSH or Protein H).

Hyperacylation of enzyme CPS1 is known to inhibit its activity in the urea cycle, resulting in hyperammonemia. A common symptom of MMA is hyperammonemia, and the inventors discovered hyperacylated CPS1 enzyme in MMA patient liver tissue samples compared to controls. Another common symptom of OAs is ketotic hyperglycinemia (KH) which results from reduced functionality of the glycine cleavage pathway. Although OA associated KH was first characterized in the 1960s, the underlying mechanism of glycine cleavage pathway inactivation was still unknown. The inventors determined that aberrant acylation of GSCH in MMA inhibits its protein activity leading to inactivation of this pathway. To restore CPS1 function, GCSH function, and the function of other hyperacylated metabolic enzyme targets in MMA and other OAs, a novel acylation resistant mutant SIRT5 protein was generated. This novel SIRT5 construct includes four lysine sites mutated to the deacylated lysine (K) mimic residue, arginine (R). This novel mutant SIRT5 protein is active (e.g., can remove methylmalonyl PTMs from pre-acylated BSA lysines in vitro), even when hyperacylated itself. Expression of the novel mutant SIRT5 protein in vivo in MMA mice increased body weight, diminished methylmalonylation, and reduced blood ammonia levels. This novel mutant SIRT5 protein can therefore be used as a therapeutic for OA patients. Provided herein is an isolated mutant SIRT5 protein having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 4 or SEQ ID NO: 10, wherein the mutant SIRT protein retains an arginine at one or more of positions 79, 112, 148, and 152 of SEQ ID NO: 4 or SEQ ID NO: 10. In some examples, an isolated mutant SIRT5 protein having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 4 or SEQ ID NO: 10 retains the arginine at positions 79, 112, 148, and 152 of SEQ ID NO: 4 or SEQ ID NO: 10. In some examples, an isolated mutant SIRT5 protein comprises or consists of the protein sequence of SEQ ID NO: 4 or SEQ ID NO: 10. In some examples, an isolated mutant SIRT5 protein further includes a purification tag, such as a FLAG-, GST- or Myc-tag (e.g., see SEQ ID NO: 10), or other molecule (e.g., an immunoglobulin Fc domain). In some examples, an isolated mutant SIRT5 protein further includes a cell penetrating peptide. Variants of the disclosed isolated mutant SIRT5 proteins, which retain an arginine at one or more of positions 79, 112, 148, and 152 of SEQ ID NO: 4 or SEQ ID NO:

10 (such as retains arginine at all four of positions 79, 112, 148, and 152 of SEQ ID NO: 4 or SEQ ID NO: 10), can be further modified, for example to include one or more additional amino acid substitutions, deletions, and/or additions, such as one or more conservative substitutions, but retain deacylation activity (e.g., ability to remove propionylation, methylmalonylation and/or malonylation from substrate) when hyperacylated.

Also provided are isolated nucleic acid molecules that encode a disclosed mutant SIRT5 protein. Such nucleic acid molecules can be DNA or RNA, such as cDNA and mRNA, as well as modified forms thereof, such as peptide nucleic acids. In some examples, the isolated nucleic acid molecule has at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 3 or SEQ ID NO: 9, and encodes an arginine at one or more of positions 79, 112, 148, and 152 of SEQ ID NO: 4 or SEQ ID NO: 10, such as encodes an arginine at all of positions 79, 112, 148, and 152 of SEQ ID NO: 4 or SEQ ID NO: 10. The disclosed mutant SIRT5 protein coding sequences can be operably linked to a promoter, such as a constitutive or inducible promoter. In some examples, the promoter is a tissue-specific promoter or organelle-specific promoter, such as a mitochondrial-specific promoter (e.g., the light-strand promoter (LSP) or the heavy-strand promoter (HSP) of the mitochondrial genome). Also provided are vectors that include a mutant SIRT5 protein coding sequence, such as a plasmid or viral vector (e.g., adeno-associated virus (AAV) or lentivirus). Also provided are host cells that include such vectors or include a mutant SIRT5 protein coding sequence (for example, as part of a lipid nanoparticle (LNP)), such as a bacterium, mammalian cell (e.g., human cell, such as a cell of a subject with OA, such as MMA or PA) or yeast cell. Also provided are compositions that include one or more of the disclosed mutant SIRT5 proteins, or one or more nucleic acid molecules encoding a mutant SIRT5 protein (such as a vector or plasmid). Such a composition can further include a pharmaceutically acceptable carrier, such as water or saline. The disclosed compositions can further include one or more other therapeutic agents, such as those used to treat an OA, such as MMA, IVA, GA1, or PA. In one example, a composition further includes one or more of L-carnitine, hydroxycobalamin, vitamin B12, an antibiotic, sodium benzoate, N-carbamylglutamate or combinations thereof. In one example, a composition further includes one or more of a MMUT, MMAA, MMAB, MMACHC, MMACHD, LMBRD1, MCEE, PCCA or PCCB protein or nucleic acid molecule encoding such. In some examples, the composition is liquid. In some examples, the composition is frozen. In some examples, the composition is lyophilized The disclosed compositions can be present in a vessel, such as a glass or plastic container, such as a syringe.

The disclosure also provides methods of diagnosis and treatment. For example, provided are methods of diagnosing an OA, such as MMA, IVA, GA1, or PA. The method can include detecting or measuring one or more proteins (such as carbamoyl phosphate synthetase (CPS1), glycine cleavage system H protein (GCSH or Protein H), SIRT5, TFAM, OPA1, a protein listed in FIG. 4, or any protein that is a member of any of the protein pathways shown in FIG. 5A and/or FIG. 5B) with hyperacylation in the form of propionyl, malonyl, methylmalonyl, or other OA specific acylation in a sample from a subject having or suspected of having an OA, wherein detecting one or more proteins posttranslationally modified with hyperacylation diagnoses the subject with OA, and the response to therapy. Also provided are methods of treating an OA that include administering a therapeutically effective amount of an isolated mutant SIRT5 protein, mutant SIRT5 protein coding sequence (such as a vector expressing such as coding sequence), or a composition comprising such molecules, to a subject having OA, thereby treating the OA. In some examples, the methods of treating OA further include detecting or measuring (1) one or more proteins hyperacylated in a sample from the treated subject having or suspected of having an OA, (2) blood ammonia levels in a sample from the treated subject having or suspected of having an OA, (3) body weight of the treated subject having or suspected of having an OA, (4), methylmalonylation of a protein (such as CPS1,

GCSH, or SIRT5) in a sample from the treated subject having or suspected of having an OA, (5) methylmalonylation of any protein that is differentially modified in the disease state selected from the list of proteins in FIG. 4, or (6) any combination of (1)-(5). The methods also include monitoring an OA subject, by detecting one or more proteins hyperacylated in a sample from the subject having OA, wherein the subject having OA previously received a liver and/or kidney transplant, or a gene editing, gene addition, mRNA or enzyme replacement therapy. In some examples, the detection step includes contacting or testing the sample with an anti-acyllysine specific antibody (e.g., an anti-methylmalonyllysine antibody) for reactivity or detecting one or more proteins using mass spectrometry. In some examples, the sample analyzed is a blood sample, plasma sample, urine sample, cerebrospinal fluid sample, or liver biopsy sample.

Also provided are methods of reducing acylation post-translational modifications (PTMs) (such as methylmalonylation, malonylation, and/or propionylation) of proteins (such as CPS1, GCSH, or SIRT5) in a subject having an OA, such as MMA, IVA, GA1, or PA. Such a method can include administering a therapeutically effective amount of an isolated mutant SIRT5 protein, a mutant SIRT5 protein coding sequence (such as an adeno-associated viral (AAV) or other vector encoding a mutant SIRT5 protein), or a composition comprising such molecules, to a subject having OA, thereby reducing acylation of proteins in the subject having OA. In some examples, the methods of reducing acylation PTMs further include detecting or measuring (1) one or more proteins hyperacylated in a sample from the treated subject having or suspected of having an OA, (2) blood ammonia levels in a sample from the treated subject having or suspected of having an OA, (3) body weight of the treated subject having or suspected of having an OA, (4), methylmalonylation of a protein (such as CPS1, GCSH, or SIRT5) in a sample from the treated subject having or suspected of having an OA, (5) methylmalonylation of any protein that is differentially modified in the disease state selected from the list of proteins in FIG. 4, or (6) any combination of (1)-(5).

The disclosed methods can further include administering to the OA subject a therapeutically effective amount of a MMUT, MMAA, MMAB, MMACHC, MMACHD, LMBRD1, or MCEE enzyme (e.g., enzyme replacement therapy), nucleic acid encoding the enzyme, such as a gene therapy vector encoding the enzyme; a low-protein high calorie diet; a diet that avoids isoleucine, valine, threonine, and methionine; L-carnitine; hydroxocobalamin; vitamin B12; one or more antibiotics; sodium benzoate; N-carbamylglutamate; or combinations thereof.

Also provided are kits. In some examples, the kit includes an isolated SIRT5 protein or nucleic acid molecule encoding a SIRT5 protein (such as a recombinant wildtype (WT) SIRT5 or the mutant SIRT5 K4R), ultra-pure, non-acylated BSA and ultra-pure acylated BSA, nicotinamide (NAM); nicotinamide adenine dinucleotide (NAD+); and an anti-methylmalonyllysine antibody (such as a polyclonal antibody generated from any one of the following peptide sequences: KKAKNKQLGHEEDYALGKD (SEQ ID NO: 42), KKKEKEVKK (SEQ ID NO: 43), KTAHIVLEDGTKMKG (SEQ ID NO: 44), KISLPHPMEIGENLDGTLKSRKRRK (SEQ ID NO: 45), KKKNDFEQGELYLKE (SEQ ID NO: 46), KDKYKQIFLGGVDKR (SEQ ID NO: 47), KGKKLVKKKIGKKDAGKKEGKC (SEQ ID NO: 48), KKNSEGLLKNKEKNQKL (SEQ ID NO: 49), KDAYIKKQNLEKA (SEQ ID NO: 50), KAFKNKETLIIEPEKN (SEQ ID NO: 51), KDVEKKLNKVTKF (SEQ ID NO: 52), KELGEKISQLKDELKT (SEQ ID NO: 53), KKIVAENHLKKI (SEQ ID NO: 54), RKKVETEAKIKQKL (SEQ ID NO: 55), KKETKGPAAENLEAKPVQAPTVKKAEKD (SEQ ID NO: 56), KKFGGQDIFMTEEQKKYYNAMKKL (SEQ ID NO: 57), KKDTQTKSIISETSNKIDTEIASLKTLMESSKL (SEQ ID NO: 58), KLGKMDRVVLGWTAVFWLTAMVEGLQVTVPDKKK (SEQ ID NO: 59), KYKIKTIQDLVSLKE (SEQ ID NO: 60), and KRKMRKGQHLDLKA (SEQ ID NO: 61)). The components of the kit can each be present in a vessel, such as a glass or plastic container, such as a syringe. Such a kit can further include one or more other therapeutic agents, such as those used to treat an OA, such as MMA, IVA, GAl, or PA. In one example, a kit further includes one or more of L-carnitine, hydroxycobalamin, vitamin B12, an antibiotic, sodium benzoate, N-carbamylglutamate or combinations thereof. In one example, a kit further includes one or more of a MMUT, MMAA, MMAB, MMACHC, MMACHD, LMBRD1, MCEE, PCCA or PCCB protein or a nucleic acid molecule encoding such.

The foregoing and other objects and feature of the disclosure will become more apparent from the following detailed description, which proceeds with reference to the accompanying figures.

### BRIEF DESCRIPTION OF THE DRAWINGS

FIG. 1 is a schematic drawing showing a summary of posttranslational modifications (PTMs), specifically acylations in MMA. The massive concentration of methylmalonyl-CoA and propionyl-CoA in cells of MMA patients leads to the non-enzymatic formation of acylated-lysine. Removal of these PTMs from targets (e.g., CPS1) by SIRT(s) maintains homeostasis.

FIGS. 2A-2D are Western blots showing PTMs in isolated MMA. (FIG. 2A) Western blots of fatty acid free, nonacetylated, ultra-pure BSA following incubation with increasing concentrations of methylmalonyl-CoA (MM-CoA) under alkaline conditions were immunoblotted with an anti-malonyllysine antibody which was observed to exhibit bi-specificity with methylmalonyllysine (top), but anti-acetyllysine antibody (middle) and anti-succinyllysine antibody (bottom) did not demonstrate bi-specificity to methylmalonyllysine. Westerns were ponceau stained for total BSA levels. (FIG. 2B) Western blot of hepatic extracts from two Mmut^{+/-}Tg^{INS-MCK-Mmmut} (Mmut control) and two Mmut^{-/-}Tg^{INS-MCK-Mmut} (Mmut mutant or MMA) mice as well as two control human hepatic extracts (C5/C28) and two MMA patient hepatic extracts (P4/P5). Stains for methylmalonyl/malonylation and β-actin. Numerous bands in the MMA mice and MMA patient liver extracts, not seen in the controls or seen at much higher levels, are present. These heavy staining bands represent proteins that have been aberrantly modified. (FIG. 2C) Western blot of hepatic extracts from two Acsf3^{(+/-)} (Acsf3 control) mice, two Acsf3^{-/-} (Acfs3 mutant) mice, two Mmut^{+/-}Tg^{INS-MCK-Mmmut} (Mmut control) mice, and two Mmut^{-/-}Tg^{INS-MCK-Mmut} (Mmut mutant or MMA) mice. Stains for methylmalonyl/malonylation and β-actin. Numerous bands in the MMA mice are seen at much higher levels than observed in the controls or Acsf3 deficient mice. These heavy staining bands represent proteins that have been aberrantly modified. (FIG. 2D) Western showing CPS1 from hepatic extracts of three control livers and three MMA patient livers (top) with corresponding immunoprecipitation using an anti-CPSlor anti-IgG Rabbit antibody (bottom). Stains for CPS1, β-actin, MMUT, and methylmalonylation/malonylation. Only the MMA patients show aberrantly modified CPS1.

FIG. 3 is a schematic drawing showing the method used to purify malonylated and methylmalonylated proteins from MMA and control mouse hepatic extracts using the bi-reactive anti-malonyllysine/methylmalonyllysine antibody used in FIGS. 2A-2D. The antibody was covalently bound to agarose beads to generate affinity columns. Hepatic lysates from wildtype (WT) and Mmut^{-/-}Tg^{INS-MCK-Mmut} mice were incubated on separate columns to purify malonylated and methylmalonylated proteins. Modified proteins were eluted from columns under acidic conditions and subjected to tandem mass spectrometry analysis. Malonylated peptides were differentiated from methylmalonylated peptides by mass shift on tandem mass spectrometry analysis. There was a greater enrichment of both modifications in Mmut^{-/-}Tg^{INS-MCK-Mmut} lysate compared to WT.

FIGS. 4A-4B is a table showing malonylated and methylmalonylated proteins from the hepatic extracts of Mmut^{-/-}; Tg^{INS-MCK-Mut} and control mice. Eight proteins (Aass, Acaa2, Atp5po, Ccdc40, Glud1, Plin4, Rida, Slc25a5 and Tsks) were both malonylated and methylmalonylated.

FIGS. 5A-5G include a DAVID GO analysis of murine MMA malonylome (FIG. 5A) and murine MMA methylmalonylome (FIG. 5B) with targeted validation of several targets to show aberrant acylation in MMA by Western and IP. DAVID GO is a gene ontology Database for Annotation, Visualization, and Integrated Discovery to which the list of hyperacylated proteins found in MMA liver tissue by mass spectrometry analysis was uploaded to determine which protein pathways would be most affected by aberrant PTMs. The urea cycle enzyme CPS1 was modified in both Mmut^{-/-}; Tg^{INS-MCK-Mut} hepatic extracts (FIG. 5C) and liver extracts from patients with MMA (FIG. 5D). Mass spectrometry analysis revealed increased methylmalonylation on Krebs cycle proteins Mdh2, Idh2, Dlst, and Aco2 and all of these proteins along with Ogdh had increased total levels in hepatic extracts from Mmut^{-/-}; Tg^{INS-MCK-Mut} mice compared to control mice (FIG. 5E). Follow up immunoprecipitation analysis confirmed increased malonylation/methylmalonylation on one such protein Idh2 in MMA mice and not controls (FIG. 5F). Other important metabolic enzymes, such as Glud 1, were modified in Mmut^{-/-}; Tg^{INS-MCK-Mmut} hepatic extracts (FIG. 5G).

FIG. 6 depicts the results of an exemplary protein, carbamoylphosphate synthase 1 (CPS1), a critical enzyme in the urea cycle and nitrogen metabolism, that was identified by immunoprecipitation using the bifunctional anti-body, followed by tandem mass spectrometry. CPS1 was modified with both malonyllysine/methylmalonyllysine on 15 lysine sites in Mmut^{-/-}; Tg^{INS-MCK-Mmut} and each modification type appeared to have preference for specific lysines on the protein structure as demonstrated in the figure. The modified lysines on the CPS1 crystal structure 5DOT obtained from RCSB are highlighted.

FIG. 7 shows Western blots of the various subunits of the glycine decarboxylase (GLDC) enzyme complex/glycine cleavage pathway proteins P (GLDC), L (DLD), T (AMT) and H (GCSH) in hepatic extracts of controls of MMA patients.

FIG. 8 is a Western blot of enzymes that are lipoylated in liver extracts from controls compared to MMA patients. Protein H (GCSH) lipoylation appears to be selectively reduced in the MMA patient liver extracts.

FIG. 9 shows Western blots obtained following immunoprecipitation showing specific loss of lipoylation on Protein H (GCSH). In the panel on the right, hepatic extracts from controls compared to MMA patients were probed using the corresponding antibodies. Protein H (GCSH) levels increased in total amount in the patient liver extracts compared to controls. The same hepatic lysates were immunoprecipitated for Protein H (GCSH) and stained for lipoic acid (left panel — top). The blot was then stripped and reprobed for Protein H (GCSH) to confirm that Protein H (GCSH) was lipoylated in control but not MMA patient samples.

FIG. 10 shows Western blots obtained following immunoprecipitation and demonstrate that loss of lipoylation on Protein H (GCSH) was caused by an acylation PTM. Hepatic extracts from controls and MMA patients were analyzed by Western blot (right) and immunoprecipitated using an anti-GCSH antibody followed by staining with anti-malonyl/methylmalonyl antibody (left). The left panel shows that Protein H (GCSH) is aberrantly modified, presumably by methylmalonylation, only in the MMA patient samples.

FIG. 11 shows a model of the inactivation of Protein H (GCSH) depicted as a ribbon (RCSB 2EDG) by methylmalonylation at the active site lysine residue that is normally lipoylated.

FIGS. 12A-12E show the results of targeted exploration of methylmalonylation of targets that control mitochondrial DNA replication, morphology, and the resulting effects on mitochondrial DNA copy number in MMA mice and patients with MMA. It was hypothesized that aberrant acylation of enzymes responsible for mtDNA stability, transcription and copy number as well as enzymes involved in the maintenance of mitochondrial structure would lead to reduced protein functionality and account for these disease phenotypes. Mitochondrial transcription factor A (Tfam), which maintains mtDNA copy number and regulates the expression of mtDNA genes, such as electron transport chain subunits, was examined first. To determine if Tfam was aberrantly modified, immunoprecipitation experiments were performed against Tfam from both hepatic and renal tissues from MMA mice and controls. In both instances, increased malonylation/methylmalonylation was observed on Tfam purified from MMA mice but not control mice (FIGS. 12A-12B). Also observed was increased co-immunoprecipitation of mitochondrial DNA-directed RNA polymerase (Polrmt), an essential mtDNA transcription enzyme recruited to mtDNA promoters by Tfam where it forms a transcription complex with Tfam, and mitochondrial transcription factor B2 (Tfb2m), in MMA mouse hepatic extracts compared to controls (FIG. 12A). Additionally, it was discovered that Polrmt exhibits increased propionylation in MMA mice compared to controls (FIG. 12A). Reduced function of Tfam and Polrmt would in turn lead to reduced mtDNA copy number as well as reduced transcription of mtDNA encoded enzymes such as those that compose portions of the electron transport chain. To examine this possibility, mtDNA copy number was examined in both human and mouse MMA hepatic extracts compared to controls (FIGS. 12C-12D). Both human and mouse MMA tissue exhibited significantly reduced mtDNA copy number compared to their respective controls, which was determined by performing real-time PCR (RT-PCR) for L strand encoded ND6 and/or H strand encoded COXI, further indicating aberrant MMA specific acylation of mitochondrial regulatory proteins could contribute to pathophysiology (FIGS. 12C-12D). Optic atrophy 1 (OPA1), a dynamin-related GTPase, which regulates mitochondrial fission and fusion, was also studied. Long isoforms of Opal demonstrated increased propionylation while short isoforms exhibited increased malonylation/methylmalonylation in MMA mice compared to controls (FIG. 12E).

FIGS. 13A-13B show Western blots of an in vitro assay of (FIG. 13A) SIRT1 or (FIG. 13B) SIRT5 enzymatic activity against methylmalonylated BSA substrate. Purified SIRT1 or SIRT5 was incubated with methylmalonylated BSA, with and without the SIRT co-factor, NAD+, and with or without the SIRT inhibitor nicotinamide, (NAM). Lane 3 (*) and lane 4 (*) show that the respective SIRTs can remove the methylmalonyltion from BSA.

FIG. 14 is a Western blot showing SIRT5 mediates de-propionylation activity using an in vitro assay of SIRT5 enzymatic activity against propionylated BSA substrate. SIRT5 was pre-modified with or without propionyl groups (propionylated-SIRT5 condition in boxes), with and without co-factor NAD+, and with or without inhibitor nicotinamide (NAM). As shown in lane 3, SIRT5 mediates the depropionylation of BSA, however, when SIRT5 is propionylated in vitro prior to incubation with substrate (lane 4), (SIRT5 propionylation appears as a band in the bottom panel in lane 4) the activity is impaired.

FIG. 15 is a flow chart providing an overview of the method used to identify lysine residues in SIRT5 for mutation, resulting in K4R SIRT5 (SEQ ID NO: 4, containing point mutations K79R, K112R, K148R, and K152R).

FIGS. 16A-16B show SIRT1 and SIRT5 activity against methylmalonylation and hyperacylation and effect of mutagenesis. (FIG. 16A) In vitro assay of SIRT1 or SIRT5 enzymatic activity against methylmalonylated BSA substrate. SIRT1 or SIRT5 was pre-modified with or without methylmalonyl groups (methylmalonylated-SIRT1/SIRT5 condition in boxes), with and without co-factor NAD+, and with or without inhibitor nicotinamide (NAM). Hypermethylmalonylation of either SIRT inhibits deacylation activity on methylmalonated BSA. (FIG. 16B) In vitro assay of SIRT5 K4R enzymatic activity against methylmalonylated BSA substrate. Methylmalonylation does not affect the activity of SIRT5 K4R despite being hypermethylmalonylated.

FIG. 17 shows the results of a modified in vitro deacylation assay using SIRT5 K4R on MMA mice (Mmut^{-/-}; Tg^{INS-MCK-Mmut}, and control mice (Mmut^{-/-}; Tg^{INS-MCK-Mmut} ) hepatic tissue lysates (SEQ ID NO: 4). The removal of methylmalonylation and propionylation of proteins in the extracts incubated with SIRT5 K4R.

FIGS. 18A-18G show effects of SIRT5 K4R-FLAG treatment. (FIG. 18A) Four of the six SIRT5 K4R-FLAG treated Mmut^{-/-}; Tg^{INS-MCK-Mut} mice exhibited hight levels of SIRT5 K4R-FLAG expression via Western blot analysis of their liver tissue extract. (FIG. 18B) Four mice demonstrated a significant increase in percent body weight compared to the GFP-treated Mmut^{-/-}; Tg^{INS-MCK-Mut} control mice as determined by student t-test (* P-value <0.05) indicating SIRT5 activity lessened disease phenotype via reversal of excessive MMA specific PTMs. Methylmalonylation and propionylation from hepatic extracts of representative Mmut^{-/-}; Tg^{INS-MCK-Mmut} mice treated with SIRT5 K4R-FLAG show reduced global methylmalonylation (FIG. 18C) but not propionylation (FIG. 18D) compared to GFP-treated Mmut^{-/-}; Tg^{INS-MCK-Mmut} controls indicating SIRT5 K4R-FLAG is capable of removing methylmalonylation but not propionylation in vivo. SIRT5 K4R-FLAG treatment led to reduced aberrant methylmalonylation of Cpsl through immunoprecipitation and Western blot analysis on hepatic extracts from SIRT5 K4R-FLAG and GFP-treated control mice (FIG. 18E) which led to reduced levels of blood ammonia levels (FIG. 18F) in SIRT5 K4R-FLAG-treated Mmut^{-/-}; Tg^{INS-MCK-Mumt} mice as compared to GFP—treated Mmut^{-/-}; Tg^{INS-MCK-Mumt} mice as well as untreated Mmut^{-/-}; Tg^{INS-MCK-Mumt} (**P<0.01, *P<0.05). Reduced acylation of Cpsl is known to increase enzymatic activity and upregulate the urea cycle. SIRT5 K4R-FLAG treatment rescued lipoylation of H-protein in the livers of Mmut^{-/-}; Tg^{INS-MCK-Mumt} mice compared to GFP-treated controls (FIG. 18G).

### SEQUENCE LISTING

The nucleic and amino acid sequences are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on Apr. 19, 2021, 65.2 KB, which is incorporated by reference herein. In the accompanying sequence listing:

SEQ ID NOS: 1 and 2 provide exemplary human SIRT5 nucleic acid and protein sequences, respectively. Source: GenBank Accession Nos. NM_001376803.1 and NP_001363732.1. Coding sequence nt 219-1151. Mature peptide amino acids 37-310.

SEQ ID NOS: 3 and 4 provide exemplary SIRT5 K4R mutant nucleic acid and protein sequences, respectively.

SEQ ID NOS: 5 and 6 provide exemplary human CPS1 nucleic acid and protein sequences, respectively. Source: GenBank Accession Nos. Y15793.1 and CAA75785.1.

SEQ ID NOS: 7 and 8 provide exemplary SIRT5 nucleic acid and protein sequences containing a FLAG tag at the C-terminus, respectively.

SEQ ID NOS: 9 and 10 provide exemplary SIRT5 K4R mutant nucleic acid and protein sequences containing a FLAG tag at the C-terminus, respectively.

SEQ ID NO: 11 provides an exemplary AAV sequence encoding the SIRT5 K4R mutant under the control of the TBG (thyroid binding globulin) promoter in an AAV backbone. Also designated as AAV8 TBG SIRT5 K4R.

SEQ ID NO: 12 provides an exemplary AAV sequence in a chicken beta actin backbone encoding the SIRT5 K4R mutant. Also designated as AAV CBA SIRT5 K4R.

SEQ ID NOS: 13-14 provide forward and reverse primers, respectively, for introducing a K79R substitution into human SIRT5.

SEQ ID NOS: 15-16 provide forward and reverse primers, respectively, for introducing a K112R substitution into human SIRT5.

SEQ ID NOS: 17-18 provide forward and reverse primers, respectively, for introducing K148R and K1524 substitutions into human SIRT5.

SEQ ID NOS: 19-21 are amino acid sequences of protein tags.

SEQ ID NOS: 22-41 are amino acid sequences of cell-penetrating peptides.

SEQ ID NOS: 42-61 are amino acids sequences of peptides used to generate polyclonal methylmalonyllysine-specific antibodies.

### DETAILED DESCRIPTION

Unless otherwise noted, technical terms are used according to conventional usage. Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Rédei, Encyclopedic Dictionary of Genetics, Genomics, and Proteomics, 2nd Edition, 2003 (ISBN: 0-471-26821-6).

The singular forms “a,” “an,” and “the” refer to one or more than one, unless the context clearly dictates otherwise. For example, the term “comprising a protein” includes single or plural proteins and is considered equivalent to the phrase “comprising at least one protein.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety, as are the GenBank® Accession numbers (for the sequence present on May 7, 2020). In case of conflict, the present specification, including explanations of terms, will control. In addition, the materials, methods, and examples are illustrative only and not intended to be limiting.

Except as otherwise noted, the methods and techniques of the present disclosure are generally performed according to conventional methods and as described in various general and more specific references that are cited and discussed throughout the present specification. See, e.g., Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999.

Administration: To provide or give a subject an agent, such as a mutated SIRT5 protein, or nucleic acid encoding such, by any effective route. Exemplary routes of administration include, but are not limited to, oral, injection (such as subcutaneous, intramuscular, intradermal, intraperitoneal, intravenous, and intratumoral), sublingual, rectal, transdermal, intranasal, vaginal and inhalation routes. In one example, administration is injection into the liver. In one example, administration is systemic. In one example, administration is local, for example into the liver.

Antibody: A polypeptide ligand comprising at least a light chain or heavy chain immunoglobulin variable region which specifically recognizes and binds an epitope of an antigen, such as a protein containing malonyllysine and/or methylmalonyllysine. Antibodies are composed of a heavy and a light chain, each of which has a variable region, termed the variable heavy (V_{H}) region and the variable light (V_{L}) region. Together, the VH region and the VL region are responsible for binding the antigen recognized by the antibody.

Antibodies include portions of antibodies, such as those not having an Fc region, such as Fab fragments, Fab″ fragments, F(ab′)₂ fragments, CH2 deleted Ab, single domain V-region Ab, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, IL); Kuby, J., Immunology, 3^{rd} Ed., W. H. Freeman & Co., New York, 1997.

In some examples, antibodies include immunoglobulins that have an Fc region that is mutated or even deleted to substantially decrease the function of the Fc region. In some examples, the mutation decreases the function of the Fc region, such as an ability to bind to Fcy receptor, by at least 50%, at least 75%, at least 80%, at least 90%, at least 95%, at least 99%, or 100% as compared to the function of the Fc region without the mutation.

Typically, a naturally occurring immunoglobulin has heavy (H) chains and light (L) chains interconnected by disulfide bonds. There are two types of light chain, lambda (X) and kappa (k). There are five main heavy chain classes (or isotypes) which determine the functional activity of an antibody molecule: IgM, IgD, IgG, IgA and IgE.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs.” The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species, such as humans. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “chimeric antibody” has framework residues from one species, such as human, and CDRs (which generally confer antigen binding) from another species, such as a murine antibody.

A “humanized” immunoglobulin is an immunoglobulin including a human framework region and one or more CDRs from a non-human (for example a mouse, rat, or synthetic) immunoglobulin. The non-human immunoglobulin providing the CDRs is termed a “donor,” and the human immunoglobulin providing the framework is termed an “acceptor.” In one embodiment, all the CDRs are from the donor immunoglobulin in a humanized immunoglobulin. Constant regions need not be present, but if they are, they are substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the

CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by means of genetic engineering (see for example, U.S. Pat. No. 5,585,089).

A “human” antibody (also called a “fully human” antibody) is an antibody that includes human framework regions and all of the CDRs from a human immunoglobulin. In one example, the framework and the CDRs are from the same originating human heavy and/or light chain amino acid sequence. However, frameworks from one human antibody can be engineered to include CDRs from a different human antibody. All parts of a human immunoglobulin are substantially identical to corresponding parts of natural human immunoglobulin sequences.

“Specifically binds” refers to the ability of individual antibodies to specifically immunoreact with one or more antigens, such as malonyllysine and methylmalonyllysine (e.g., in some examples an anti-malonyllysine antibody is a bispecific antibody, which specifically binds to both malonyllysine and methylmalonyllysine), relative to binding to unrelated proteins, such as one containing succinyllysine or acetyllysine. For example, a malonyllysine-specific binding agent binds substantially only to proteins containing malonyllysine and/or methylmalonyllysine in vitro or in vivo. As used herein, the term “malonyllysine-specific binding agent” includes antibodies and other agents that bind substantially only to a protein in that preparation that includes malonyllysine and/or methylmalonyllysine. In some examples, an antibody or fragment thereof (such as an anti-malonyllysine molecule) specifically binds to a target (such as a protein containing malonyllysine and/or methylmalonyllysine) with a binding constant that is at least 10³ M⁻¹ greater, 10⁴M⁻¹ greater or 10⁵ M⁻¹ greater than a binding constant for other molecules in a sample or subject. In some examples, an antibody (e.g., monoclonal antibody) or fragments thereof, has an equilibrium constant (Kd) of 1 nM or less. For example, an antibody or fragment thereof binds to a target, such as malonyllysine with a binding affinity of at least about 0.1×10⁻⁸ M, at least about 0.3×10⁻⁸M, at least about 0.5×10⁻⁸ M, at least about 0.75×10⁻⁸ M, at least about 1.0×10⁻⁸ M, at least about 1.3×10⁻⁸ M at least about 1.5×10⁻⁸M, or at least about 2.0×10⁻⁸ M, at least about 2.5×10⁻⁸, at least about 3.0×10⁻⁸, at least about 3.5×10⁻⁸, at least about 4.0×10⁻⁸, at least about 4.5×10⁻⁸, or at least about 5.0×10⁻⁸M. In certain embodiments, a specific binding agent that binds to target has a dissociation constant (Kd) of <104 nM, <100 nM, <10 nM, <1 nM, <0.1 nM, <0.01 nM, or <0.001 nM (e.g., 10⁻⁸M or less, e.g., from 10⁻⁸M to 10⁻¹³M, e.g., from 10⁻⁹M to 10⁻¹³ M). In one embodiment, Kd is measured by a radiolabeled antigen binding assay (RIA) performed with the Fab version of an antibody of interest and its antigen (see, e.g., Chen et al., J. Mol. Biol. 293:865-881, 1999). In another example, Kd is measured using surface plasmon resonance assays using a BIACORES-2000 or a BIACORES-3000 (BIAcore, Inc., Piscataway, N.J.) at 25° C. with immobilized antigen CMS chips at about 10 response units (RU). In some examples, specificity of an antibody, such as an malonyllysine antibody, is determined by in vitro staining of methylmalonylated BSA and/or by mass shift in tandem mass spectrometry analysis.

Carbamoyl phosphate synthetase (CPS1): (e.g., OMIM 608307) A ligase enzyme (EC 6.3.4.16) located in the mitochondria involved in the production of urea. CPS1 transfers an ammonia molecule from glutamine or glutamate to a molecule of bicarbonate that has been phosphorylated by a molecule of ATP. The resulting carbamate is then phosphorylated with another molecule of ATP. The resulting molecule of carbamoyl phosphate leaves the enzyme. The full-length human protein sequence encodes a 1,500-amino acid precursor polypeptide with a deduced molecular mass of 165 kD that shows 94.4% amino acid homology to the rat enzyme precursor. The 165-kD proenzyme is produced in the cytoplasm and transported into the mitochondria where it is cleaved into its mature 160-kD form. CPS1 is expressed in the liver and in epithelial cells of the intestinal mucosa. It is shown herein that in OA patients, such as subjects having MMA or PA, CPS1 includes PTMs such as methylmalonylation, which deactivates CPS1, and this

PTM is not effectively removed by SIRTS, due to hyperacylation inactivation of deacylase activity. The disclosed SIRTS mutants can effectively activate CPS1 by deactylating it.

CPS1 sequences are publically available, for example from the GenBank® sequence database (e.g., Accession Nos. CAA75785.1, NP_001139222.1, NP_058768.1, and AAI26970.1 provide exemplary CPS1 protein sequences, while Accession Nos. Y15793.1 and NM_023525.2 provide exemplary CPS1 nucleic acid sequences). One of ordinary skill in the art can identify additional CPS1 nucleic acid and protein sequences, including CPS1 variants having CPS1 activity.

Contact: Placement in direct physical association, including a solid or a liquid form. Contacting can occur in vitro or ex vivo, for example, by adding a reagent to a sample (such as one containing a mutant SIRTS protein), or in vivo by administering to a subject.

Detect: To determine if a particular agent is present or absent, and in some example further includes quantification of the agent if detected.

Effective amount or Therapeutically effective amount: The amount of agent, such as a mutated SIRTS protein (or nucleic acid encoding such) disclosed herein, that is an amount sufficient to prevent, treat (including prophylaxis), reduce and/or ameliorate the symptoms and/or underlying causes of any of a disorder or disease, such as an OA, such as MMA or PA. In one embodiment, an “effective amount” of a mutated SIRTS protein provided herein (or nucleic acid encoding such) is sufficient to reduce or eliminate a symptom of a disease, such as an OA, such as MMA or PA, for example by deacylating (e.g., de-methylmalonylating, de-propionylating, or de-malonylating) are all forms of acylation enzymes in critical intracellular pathways, including CPS1.

Glycine cleavage system H protein (GCSH or Protein H): (e.g., OMIM 238330) A shuttle protein for methylamine groups in the glycine cleavage pathway. The glycine cleavage pathway is responsible for the catabolism of glycine to CO₂ and ammonia and is regulated by four major proteins H (GCSH), P(GLDC), T(GCST) and L (DLD). To function in this pathway, GCSH is first lipoylated on lysine 107 by enzymes LIPT2 and LIAS. GLDC then decarboxylates glycine generating CO₂ and a methylamine, which is placed onto the lipoic acid PTM of GCSH. GCST then reduces this methylamine-lipoic acid group on GCSH generating ammonia. DLD then oxidizes the lipoic acid group so that GCSH can cycle back around to accept another methyl-amine from GCLD and perpetuate the cycle. Loss of function in any of these proteins results in Nonketotic Hyperglycinemia or NKH, a severe recessive genetic disorder with frequently lethal, neurological symptoms in the neonatal period. However, patients with certain OAs including propionic acidemia, methlymalonic acidemia, and isovalerica acidema, also present with a milder form hyperglycinemia known as ketotic hyperglycinemia or KH. Higher glycine levels in the blood and urine of

OA patients may be associated with worse neurological outcomes as glycine is an inhibitory neurotransmitter.

GCSH sequences are publically available, for example from the GenBank® sequence database (e.g., Accession Nos. CAG33353.1, AAH14745.1, NP_058768.1, and AAH88114.1 provide exemplary GCSH protein sequences, while Accession Nos. CR457072.1, BC014745.1 and NM_133598.2 provide exemplary

GCSH nucleic acid sequences). One of ordinary skill in the art can identify additional GCSH nucleic acid and protein sequences, including GCSH variants having GCSH activity.

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used. Thus, host cells can be transgenic, in that they include nucleic acid molecules that have been introduced into the cell, such as a nucleic acid molecule encoding a SIRTS protein disclosed herein. Exemplary host cells include mammalian cells (e.g., an immortal cell line), bacterial cells (e.g., E. coli), and yeast cells. Isolated: An “isolated” biological component (such as a mutated SIRTS protein or nucleic acid molecule) has been substantially separated, produced apart from, or purified away from other biological components in the cell of the organism in which the component naturally occurs, such as other chromosomal and extrachromosomal DNA and RNA, and proteins. Nucleic acids molecules and proteins which have been “isolated” thus include nucleic acids and proteins purified by standard purification methods. The term also embraces nucleic acid molecules and proteins prepared by recombinant expression in a host cell as well as chemically synthesized nucleic acids. A purified or isolated cell, protein, or nucleic acid molecule can be at least 70%, at least 80%, at least 90%, at least 95%, at least 96%, at least 97%, at least 98%, or at least 99% pure.

Mammal: This term includes both human and non-human mammals. Similarly, the term “subject” includes both human and veterinary subjects (such as cats, dogs, cows, and pigs) and rodents (such as mice and rats).

Metabolic disorder: A disorder that negatively alters the body's processing and distribution of nutrients such as proteins, fats, and carbohydrates. Metabolic disorders can happen when abnormal chemical reactions in the body alter the normal process to degrade nutrients from the diet. Enzymes regulate and perform the metabolism of nutrients such as proteins, fats, and carbohydrates. As such, any enzyme that is involved of branched chain amino acids, fatty acids, and organic acids can cause serious metabolic disorders including organic acidemias. The affected patients can have severe metabolic instability, growth problems, heart, kidney, muscle, bone, neurological and gastrointestinal disorders. Treatment with a SIRT5 protein is described herein to mitigate clinical symptoms and improve laboratory parameters, such as acid-base balance, ammonia levels, amino and organic acid concentrations, and lab values such as blood counts, liver and kidney function tests, seizures, growth failure, pancreatitis, and lethargy. Methylmalonic acidemia (MMA): An inborn error of metabolism wherein the body cannot break down certain proteins and fats, resulting in a buildup of toxic levels of methylmalonic acid in the blood. Also referred to as isolated MMA. This OA disrupts normal amino acid metabolism.

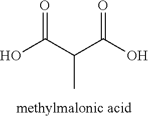

Signs and symptoms usually appear in early infancy and vary from mild to life-threatening. Affected infants can experience vomiting, dehydration, weak muscle tone (hypotonia), developmental delay, lethargy, hepatomegaly, and failure to thrive. Long-term complications can include feeding problems, intellectual disability, chronic kidney disease, and pancreatitis. Without treatment, MMA can lead to coma and death. Mutations in the MMUT (methylmalonyl-CoA mutase, EC 5.4.99.2, which can be partial (mut-) or complete (mut0) enzyme deficiency), MMAA (metabolism of cobalamin associated A), MMAB (metabolism of cobalamin associated B), MMACHC (metabolism of cobalamin associated C), MMACHD (metabolism of cobalamin associated D), LMBRD1 (lysosomal cobalamin transporter), and MCEE (methylmalonyl-CoA epimerase) genes cause different types of MMA. Currently MMA is treated with a low-protein, high-calorie diet, certain medications, antibiotics and, in some cases, organ transplantation. The present disclosure provides a novel diagnostic method and treatment for MMA, including detecting methylmalonyllysine modified enzymes (e.g., CPS1) and treatment using a mutant SIRT5 containing four mutated lysines that cannot accept acyl groups.

Operably linked A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence (such as a mutated SIRT5 coding sequence). Generally, operably linked DNA sequences are contiguous and, where necessary to join two protein coding regions, in the same reading frame.

Organic acidemias (OAs): A class of diseases characterized by loss-of-function mutations in amino acid, fatty acid, and cholesterol catabolic pathway enzymes. These inborn errors of metabolism typically arise from defects in the catabolism of amino- and fatty acids. OAs are difficult to treat and have multisystemic manifestations, including accumulation of pathway acyl-CoA intermediates, and accumulation of acylic acids (toxic metabolites), leading to increased morbidity and mortality. Examples include methylmalonic acidemia (MMA), propionic acidemia (PA), isovaleric acidemia (IVA), and glutaric acidemia type 1 (GA1).

Pharmaceutically acceptable carriers: The pharmaceutically acceptable carriers useful in this invention are conventional. Remington: The Science and Practice of Pharmacy, 22n^{d} ed., London, UK: Pharmaceutical Press, 2013) describes compositions and formulations suitable for pharmaceutical delivery of the disclosed mutated SIRT5 proteins (or nucleic acid molecules encoding such) herein disclosed. In a specific example, the carrier is water or physiological saline.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Post-translational modifications (PTM): Alterations made to a protein after the protein is synthesized, which include covalent modifications. These alterations can modify the activity of the protein (e.g., increase or decrease the activity of the modified protein). PTMs can occur on the amino acid side chains or at the protein's C- or N-termini. PTMs can alter an amino acid by modifying an existing functional group or introducing a new one, such as phosphate. Several types of PTM exist, such as phosphorylation, glycosylation, glutarylation, methylation, propionylation, lipidation, and carbonylation. Protein acylation involves the covalent placement of an acyl group to a lysine residue of a protein. Deacylation enzymes, sirtuins, control metabolism at many levels usually through deacylation of lysine residues on a multitude of enzymes. In a specific example, a PTM includes methylmalonylation.

Promoter: An array of nucleic acid control sequences that direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

Propionic acidemia (PA): An inborn error of metabolism wherein patients have non-functional propionyl-CoA carboxylase, and thus cannot convert propionyl-CoA to methylmalonyl-CoA. This results in a buildup of propionyl-CoA, propionic acid, ketones, ammonia, and other toxins in the blood and urine. This OA disrupts normal amino acid metabolism.

Signs and symptoms usually appear in immediately in newborns. Affected infants can experience poor feeding, vomiting, dehydration, acidosis, low muscle tone (hypotonia), seizures, and lethargy. The effects of PA quickly become life-threatening. Long-term complications can include chronic kidney disease, cardiomyopathy, and prolonged QTc interval. Without treatment, PA can lead to coma and death. Mutations in the PCCA or PCCB genes cause PA. Currently PA is treated with a low-protein, high-calorie diet. The protein mixture administered is devoid of methionine, threonine, valine, and isoleucine. Patients can receive L-carnitine treatment, antibiotics and, in some cases, liver transplantation. The present disclosure provides a novel diagnostic method and treatment for PA, including detecting methylmalonyllysine modified enzymes (e.g., CPS1) and treatment using a mutant SIRT5 containing four mutated lysines that cannot accept acyl groups.

Purification tag/linker A sequence of amino acids attached or linked to a protein of interest (such as a mutant SIRT5 protein), which can assist in purification or isolation of the protein of interest. Such tags include his (polyhistidine), immunoglobulin Fc domain, FLAG (DYKDDDDK; SEQ ID NO: 19), GST, 51 (NANNPDWDF; SEQ ID NO: 20), and Myc (CEQKLISEEDL; SEQ ID NO: 21) tags. In some examples, such a tag can be attached to the C-terminus or the N-terminus of the protein of interest (such as a mutant SIRT5 protein provided herein).

Recombinant: A recombinant nucleic acid molecule is one that has a sequence that is not naturally occurring (e.g., a mutated SIRT5 protein) or has a sequence that is made by an artificial combination of two otherwise separated segments of sequence. This artificial combination can be accomplished by routine methods, such as chemical synthesis or by the artificial manipulation of isolated segments of nucleic acids, such as by genetic engineering techniques. Similarly, a recombinant protein is one encoded for by a recombinant nucleic acid molecule. Similarly, a recombinant or transgenic cell is one that contains a recombinant nucleic acid molecule and expresses a recombinant protein.

Remove or Separate: To divide or move apart, for example by taking something away. Sample: Any biological specimen obtained from a subject, such as a mammalian subject, that contains nucleic acid molecules and/or proteins. Biological samples include all clinical samples useful for detection of disease (for example, an OA) in subjects, including, but not limited to, cells, tissues, and bodily fluids, such as blood; derivatives and fractions of blood (such as serum and plasma); cerebrospinal fluid; urine; biopsied or surgically removed tissue (such as a liver or kidney biopsy or sample); fine needle aspirates; sputum; and saliva.

Sequence identity of amino acid sequences: The similarity between amino acid (or nucleotide) sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of the mutated SIRTS proteins and coding sequences disclosed herein are typically characterized by possession of at least about 80%, at least 90%, at least 95%, at least 96%, at least 97%, at least 98% or at least 99% sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or at least 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided. Thus, a mutant SIRTS protein disclosed herein can have at least 80%, at least 85%, at least 90%, at least 91%, at least 92%, at least 95%, at least 96%, at least 97%, at least 98% or at least 99% sequence identity to SEQ ID NO: 4, and retains the K79R, K112R, K148R, and K152R substitutions, and has de-acylating activity and resistance to acylation inactivation (e.g., SEQ ID NO: 4 retaining the K79R, K112R, K148R, and K152R substitutions and further having one or more, such as 1, 2, 3, 4, 5, 6, 7, 8, 9, 10, 11, 12, 13, 14, 15, 16, 17, 18, 19 or 20 additional mutations, such as amino acid substitutions, deletions, additions, or combinations thereof).

Similarly, exemplary mutated SIRT5 coding sequences in some examples have at least 60%, at least 70%, at least 75%, at least 80%, at least 85%, at least 90%, at least 92%, at least 95%, at least 98%, or at least 99% sequence identity to SEQ ID NO: 3, while encoding a protein retaining the K79R, K112R, K148R, and K152R substitutions, and having de-acylating activity and resistance to acylation inactivation. Silent mating type information regulation 2 homolog (Sirtin): A class of proteins that possess either mono-ADP-ribosyltransferase, or deacylase activity, including deacetylase, desuccinylase, demalonylase, demyristoylase and depalmitoylase activity. Sirtuins are a family of signaling proteins involved in metabolic regulation. They are ancient in animal evolution and appear to possess a highly conserved structure throughout all kingdoms of life. Whereas bacteria and archaea encode either one or two sirtuins, eukaryotes encode several sirtuins in their genomes. Members of the sirtuin family are characterized by a sirtuin catalytic core domain composed of two subdomains, connected by several loops that form a binding cleft for the nicotinamide and ribose moieties of NAD+ and the acyllysine substrate. In humans, seven SIRTs have been identified, SIRT1 through SIRT7. For exemplary review see Feldman et al., J. Biol. Chem. 276:42419-27, 2012).

Sirtuin 1 (SIRT1): (e.g., OMIM 604479) Also known as NAD-dependent deacetylase sirtuin-1. Sirtuin 1 is a member of the sirtuin family of proteins, homologs of the Sir2 gene in S. cerevisiae. In humans, SIRT1 is located in the nucleus and the cytoplasm. SIRT1 de-acetylates and de-propionylates many proteins, thereby activating or deactivating the protein. SIRT1 is a regulator of glucose and fat metabolism in response to energetic challenges. SIRT1 sequences are publically available, for example from the GenBank® sequence database (e.g., Accession Nos. AAH12499.1, NP_001139222.1, and AAI52315.1 provide exemplary SIRT1 protein sequences, while Accession Nos. BC012499.1, NM_001145750.2 and NM_019812.3 provide exemplary SIRT1 nucleic acid sequences). One of ordinary skill in the art can identify additional SIRT1 nucleic acid and protein sequences, including SIRT1 variants having SIRT1 activity.

Sirtuin 5 (SIRT5): (e.g., OMIM 604483) Sirtuin 5 is a member of the sirtuin family of proteins, homologs of the Sir2 gene in S. cerevisiae. In humans, SIRT5 is located in the mitochondria. SIRT5 has desuccinylation, demalonylation, deglutarylation, and de-acetylating activity, capable of removing succinyl, malonyl, glutaryl, and acetyl groups from the lysine residues of proteins. It is shown herein that SIRT5 also has depropionylation and demethylmalonylation activity. SIRT5 de-acylates and regulates carbamoyl phosphate synthetase (CPS1), the rate-limiting and initiating step of the urea cycle in liver mitochondria. De-acylation of CPS1 stimulates its enzymatic activity.

SIRT5 sequences are publically available, for example from the GenBank® sequence database (e.g., Accession Nos. NP_001363732.1, AAH87898.1, and NP_001126552.1 provide exemplary SIRT5 protein sequences, while Accession Nos. NM_178848.3, NM_012241.5, and NM_001133080.1 provide exemplary SIRT5 nucleic acid sequences). Specific exemplary native SIRT5 sequences are shown in SEQ ID NOS: 1-2. One of ordinary skill in the art can identify additional SIRT5 nucleic acid and protein sequences, including SIRT5 variants.

In one example, a mutant SIRT5 protein is one that includes one or more mutations at native lysine residues, such as amino acid substitutions such as K to R substitutions), which (1) retain de-acylating activity, but are resistant to acylation inactivation, (2) reduce aberrant methylmalonylation, (3) reduce levels of blood ammonia in a subject with OA, or combinations of (1)-(3). One example SIRT5 mutant protein, K4R, is shown in SEQ ID NO: 4. Thus, one example, mutant SIRT5 is a variant of SIRT5 with de-acylating activity and resistance to acylation inactivation, reduces aberrant methylmalonylation, and reduces blood ammonia levels in a subject with OA (e.g., SEQ ID NO: 4 or a variant thereof that retains the K79R, K112R, K148R, and K152R substitutions, such as one having at least 90%, at least 95%, at least 96%, at least 97%, at least 98% or at least 99% sequence identity to SEQ ID NO: 4 or 10, that retains the K79R, K112R, K148R, and K152R substitutions).

Subject: Any mammal, such as humans, non-human primates, pigs, sheep, cows, dogs, cats, rodents and the like which is to be the recipient of the particular treatment, such as treatment with a mutated SIRT5 protein (or corresponding nucleic acid molecule) provided herein. In two non-limiting examples, a subject is a human subject or a murine subject. In some examples, the subject has an OA, such as an MMA (e.g., isolated MMA), or PA. In some examples, the subject has elevated methylmalonylation of proteins, such as SIRT5 and CPS1.

Transduced and Transformed: A virus or vector “transduces” a cell when it transfers nucleic acid into the cell. A cell is “transformed” or “transfected” by a nucleic acid transduced into the cell when the DNA becomes stably replicated by the cell, either by incorporation of the nucleic acid into the cellular genome, or by episomal replication.

Numerous methods of transfection are known, such as: chemical methods (e.g., calcium-phosphate transfection), physical methods (e.g., electroporation, microinjection, particle bombardment), fusion (e.g., liposomes), receptor-mediated endocytosis (e.g., DNA-protein complexes, viral envelope/capsid-DNA complexes) and by biological infection by viruses such as recombinant viruses (Wolff, J. A., ed, Gene Therapeutics, Birkhauser, Boston, USA (1994)). In the case of infection by retroviruses, the infecting retrovirus particles are absorbed by the target cells, resulting in reverse transcription of the retroviral RNA genome and integration of the resulting provirus into the cellular DNA.

Transgene: An exogenous gene supplied by a vector. In one example, a transgene includes a mutated SIRT5 coding sequence.

Vector: A nucleic acid molecule as introduced into a host cell, thereby producing a transformed host cell. A vector may include nucleic acid sequences that permit it to replicate in the host cell, such as an origin of replication. A vector may also include a mutated SIRT5 coding sequence and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

Vitamin B12 deficiency: A condition that occurs when blood and tissue levels of B12 are too low. Vitamin B12 deficiency can occur, for example, when a subject has a decreased ability to absorb vitamin B12 from the stomach or intestines, does not intake sufficient levels of B12 or has an increased requirement for B12.

### Overview

Provided herein is the identification of a new pathophysiological consequence of impaired acyl-CoA metabolism in OAs: the accumulation of aberrant posttranslational modifications (PTMs) that modify enzymes in critical intracellular pathways, especially during periods of increased stress. The OAs methylmalonic acidemia (MMA) and the related disorder propionic acidemia (PA) comprise a relatively common and heterogeneous group of inborn errors of metabolism. Most affected individuals display severe multisystemic disease characterized by metabolic instability, chronic renal disease, neurological complications, and in propionic acidemia, cardiomyopathy. The treatment of these disorders currently entails adherence to a low protein diet, carnitine supplementation and vigilant clinical monitoring. However, despite meticulous medical management, patients with MMA and PA suffer from substantial morbidity related to the disease. Thus, new treatments are needed.

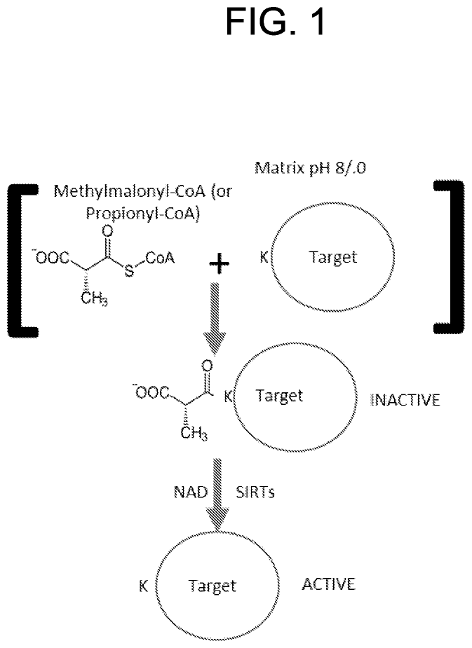

The basis for the dysregulation of critical pathways in intermediary metabolism in MMA and PA were previously unknown. Given the overabundance of mitochondrial acyl-CoA species, specifically propionyl- and methymalonyl-CoA, that form in cells as a consequence of these enzyme deficiencies, and further, that acylation in the mitochondria may be mediated through a non-enzymatic process, it was hypothesized that metabolically active cell types known to be affected by the secondary mitochondriopathy of MMA, such as hepatocytes, will exhibit excessive acylation-class posttranslational modifications (PTMs). A schematic of the relationships between PTM and SIRTs in MMA and PA is presented in FIG. 1.

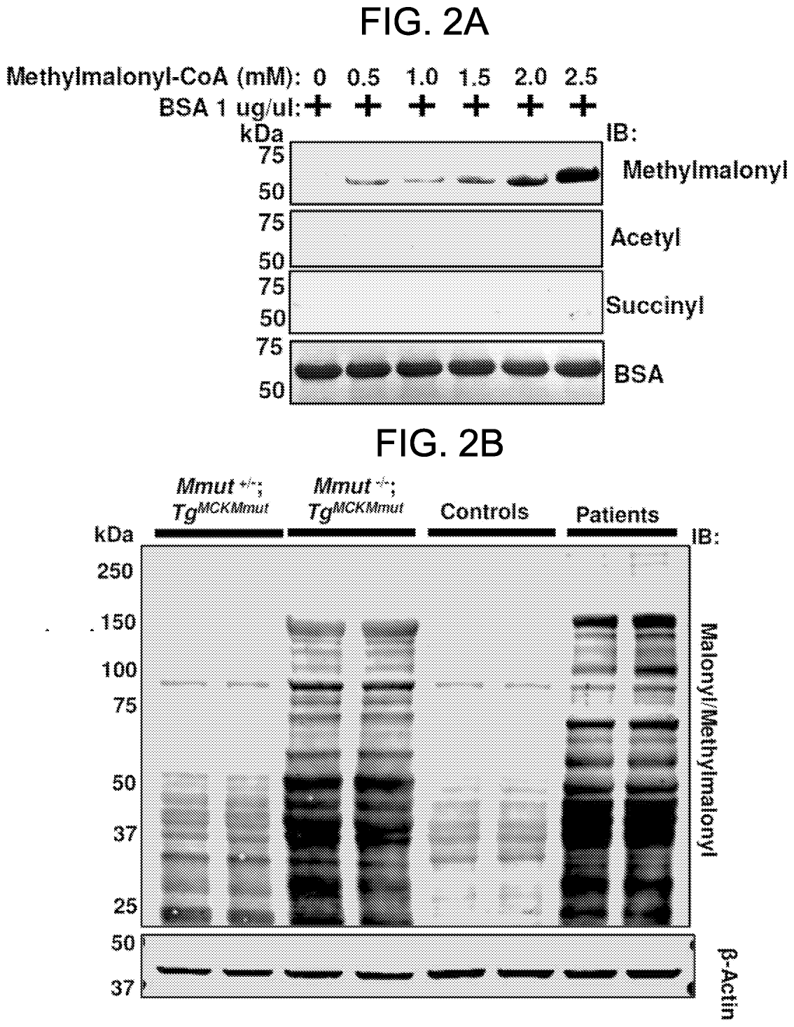

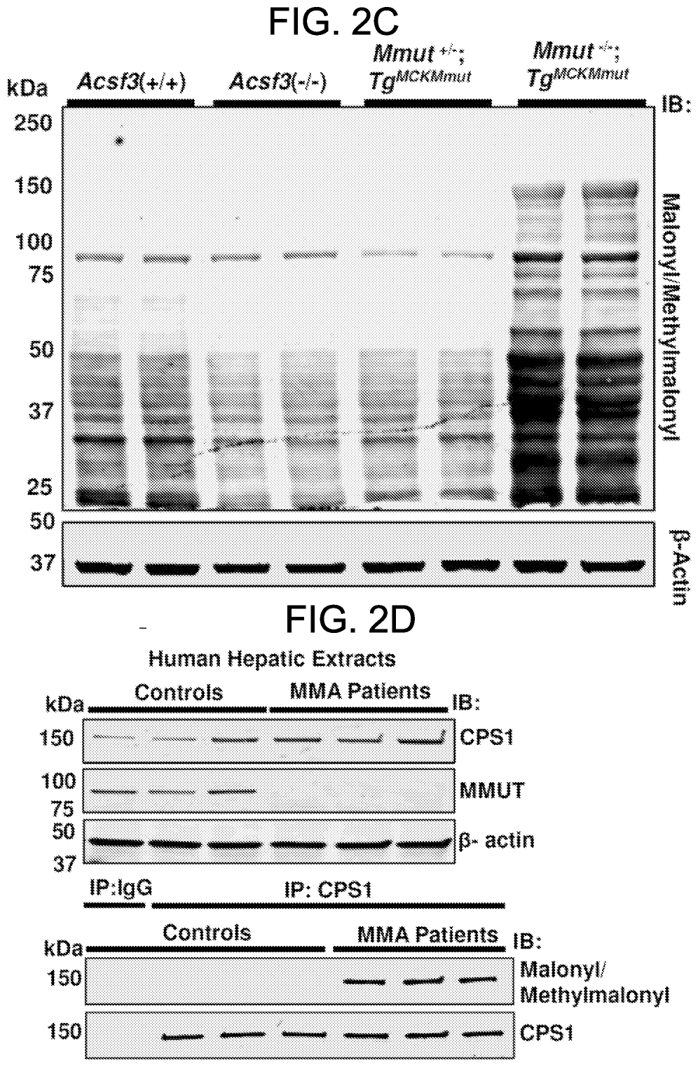

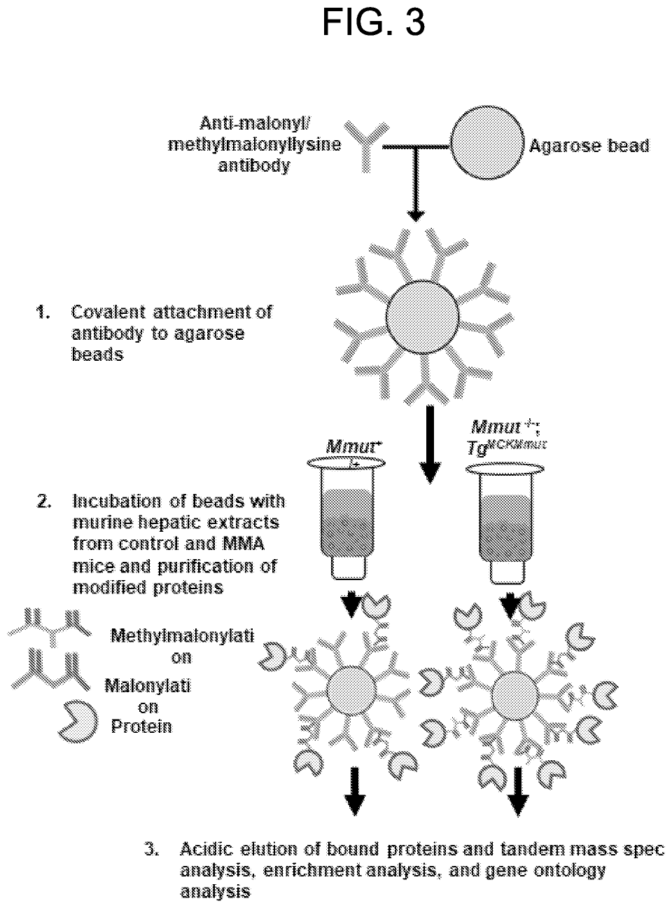

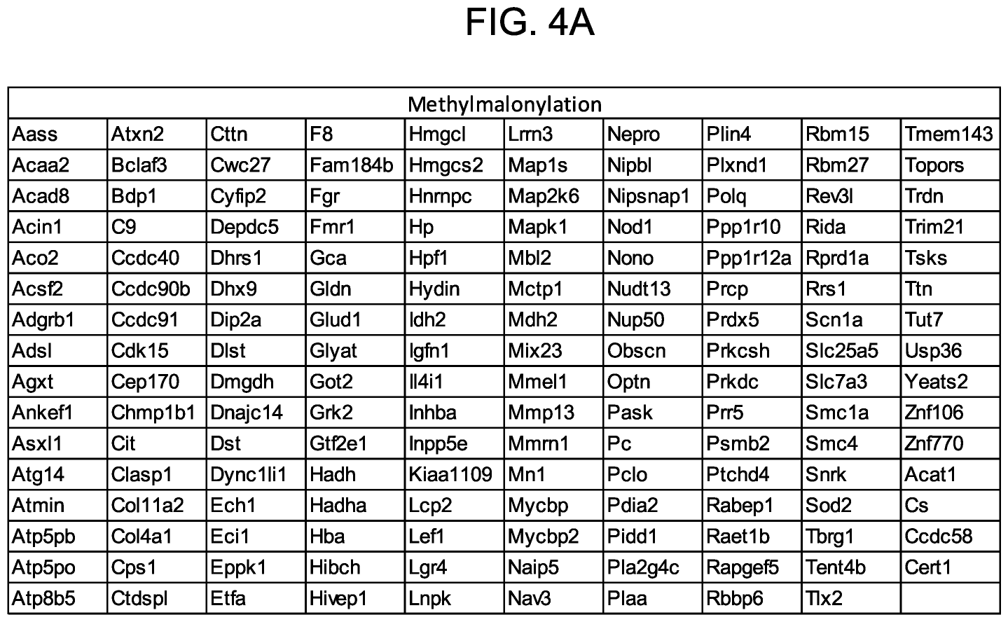

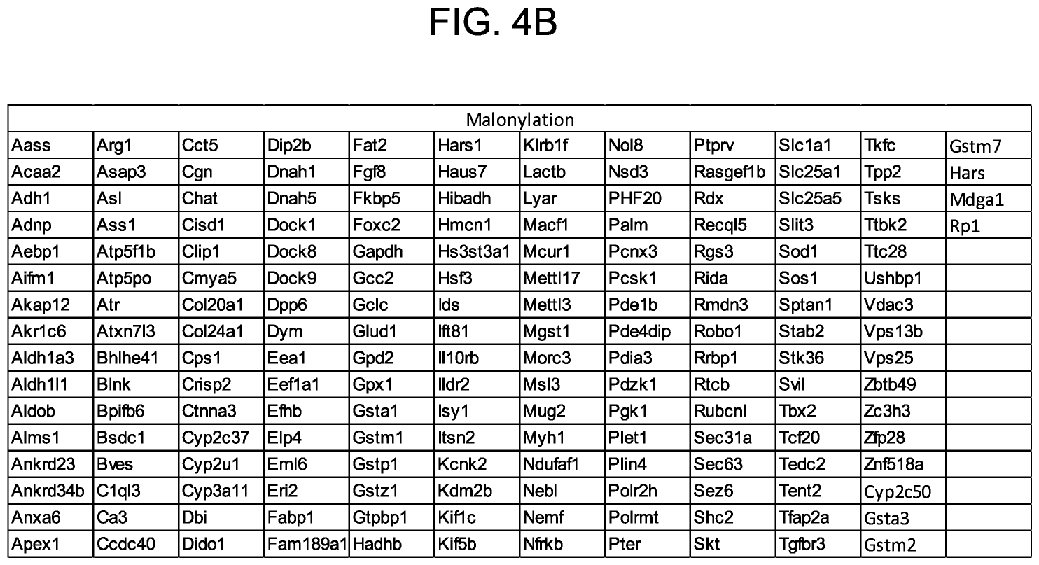

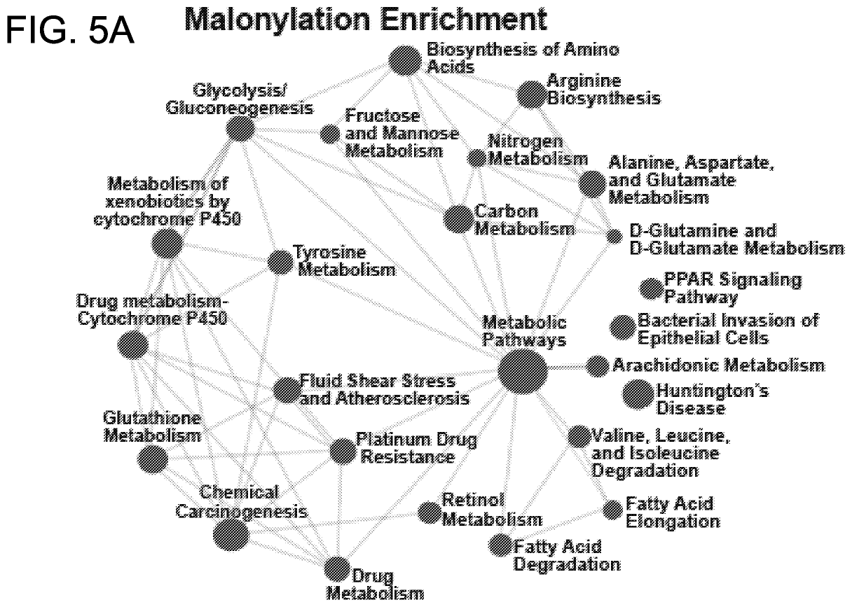

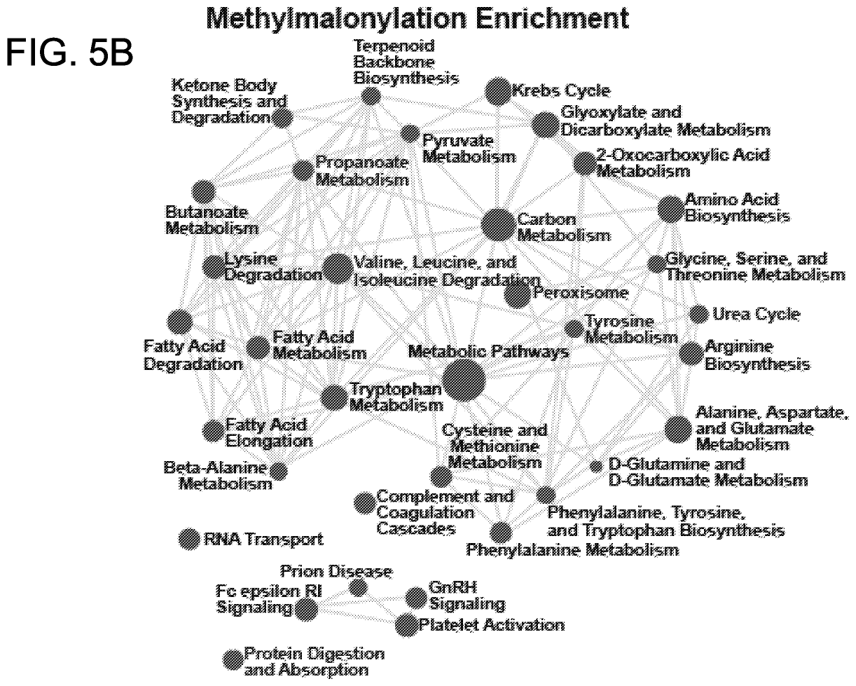

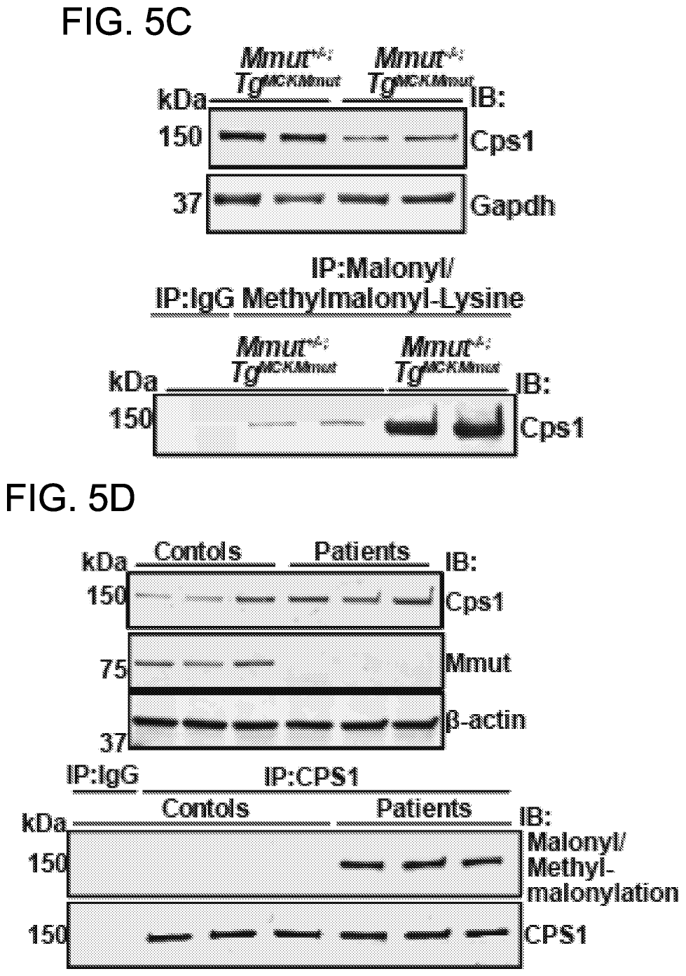

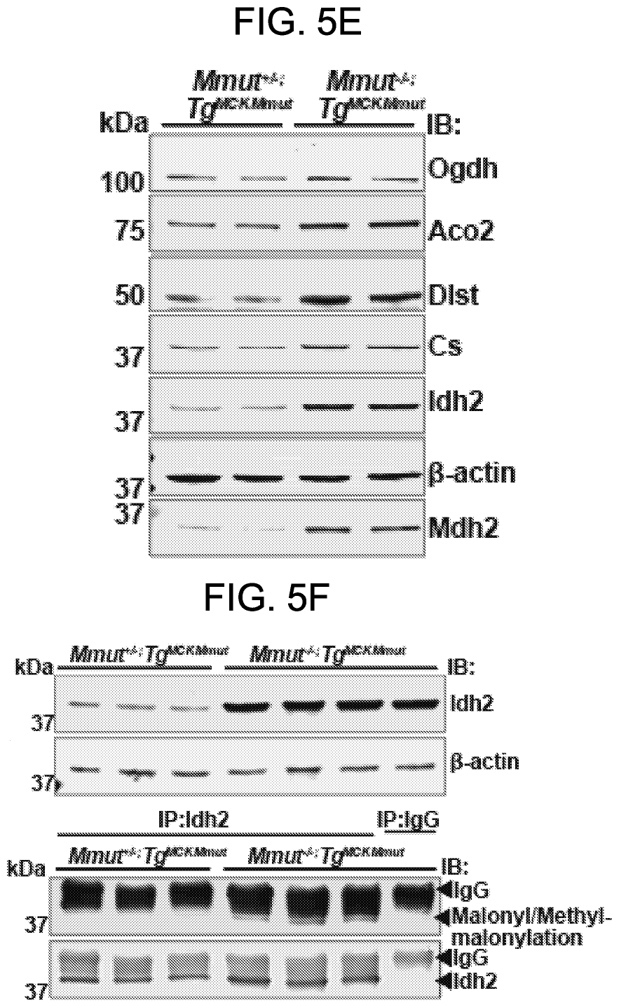

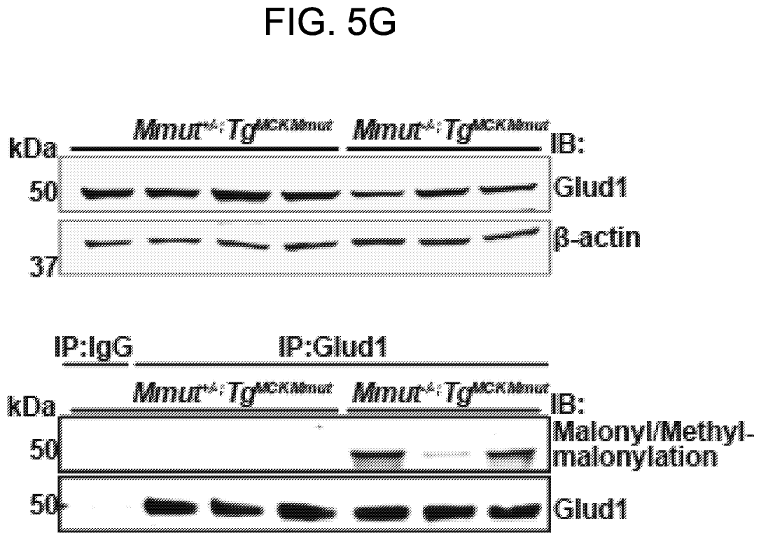

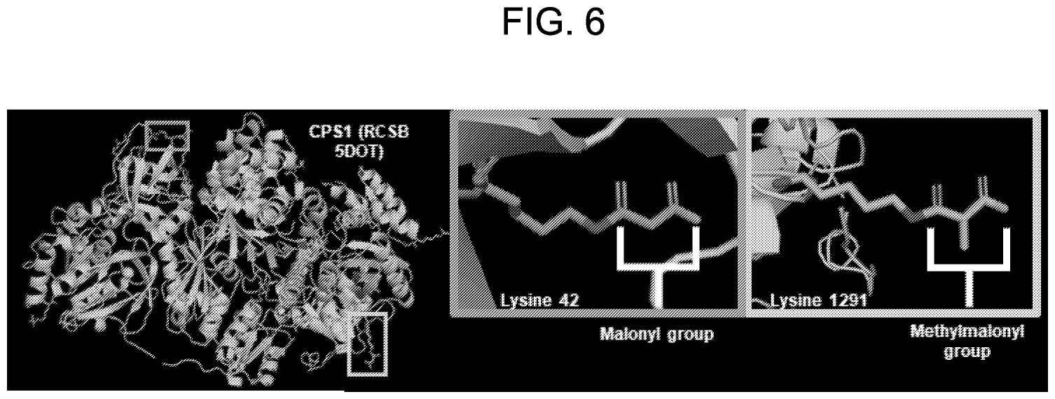

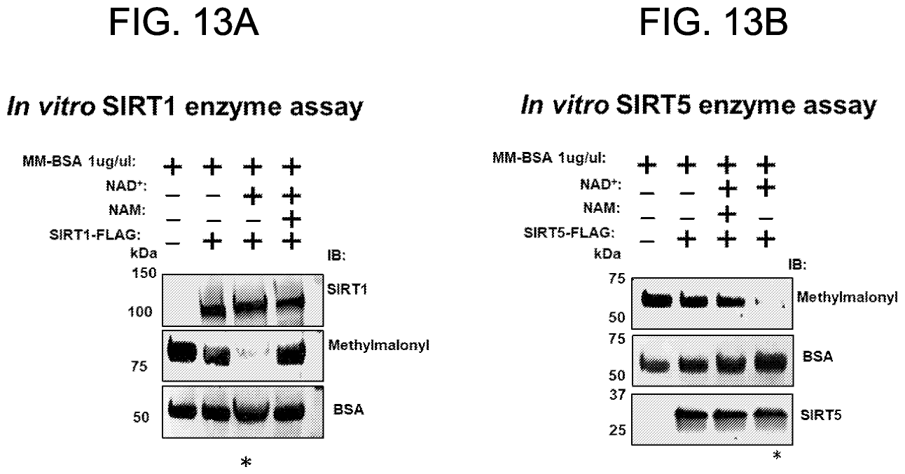

Using a mouse model that recapitulates the hepatic mitochondriopathy of MMA (Mmut^{-/-}; Tg^{INS-MCK-Mut}), and human liver tissues from MMA patients and controls, PTMs were evaluated in hepatic extracts with malonyl-lysine antibodies (FIGS. 2A-2D). Widespread hyperacylation in human and murine MMA tissues compared to controls, but not in animals with acyl-CoA synthetase family member 3 (Acsf3) deficiency, a disorder of acyl-CoA synthesis, was observed. Analysis of purified hepatic extracts from MMA and control mice using anti-PTM antibody columns and mass spectrometry to characterize the PTM proteome was performed (FIG. 3). Mass spectrometry followed by gene ontology analysis revealed excessive acylation of enzymes involved in a multitude of metabolic pathways such as glutathione, urea, arginine, lysine, tryptophan, valine, isoleucine, methionine, threonine, and fatty acid metabolism in MMA mice but not controls (FIGS. 4 and 5). This was validated via immunoprecipitation analysis and Western blotting. Immunoprecipitation experiments using MMA murine and MMA human hepatic extracts confirmed hyperacylation of urea cycle enzyme carbamoyl phosphate synthetase 1 (CPS1) compared to respective controls. Tandem mass spectrometry analysis indicated 15 sites of hyperacylation including sites associated with CPS1 inactivation (lysine 1291) and hyperammonemia (FIG. 6). Immunoprecipitation analysis also indicated novel hyperacylation of GCSH, an essential shuttle protein in the glycine cleavage pathway, which prevents placement of the activating lipoic acid PTM and pathway activity (FIGS. 11 and 13). Inactivation of GCSH has been linked to hyperglycinemia.

In addition, using nonenzymatic acylation reactions, PTM-modified BSA targets (methylmalonylated or propionylated) were generated for in vitro analyses. SIRT1-7 deacylase activity was analyzed using BSA-PTM standards to identify the SIRT(s) that most efficiently remove MMA related PTMs. SIRT5 was identified as the primary mediator of demethylmalonylation, with activity toward other PTMs, including propionylation. Because hyperacylation could be inhibitory to protein function, these observations indicate that hyperacylation of key enzymes in pathways dysregulated in MMA contributes to altered metabolism and identifies new targets for therapeutic intervention. The delineation of a PTM:SIRT axis in MMA and PA provides novel insights into disease mechanisms in MMA, and identifies a new approach, sirtuin modulation, for targeted therapies to treat all forms of MMA as well as other of the larger group of OAs and fatty acid oxidation disorders where acyl-CoA accretion occurs.

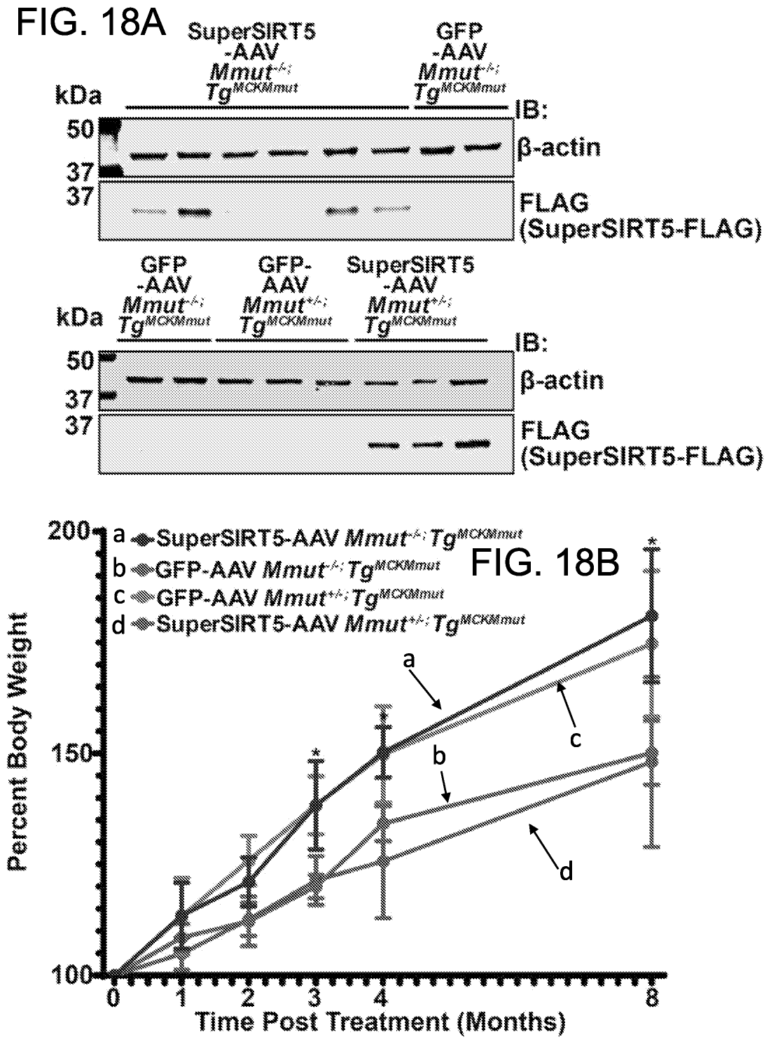

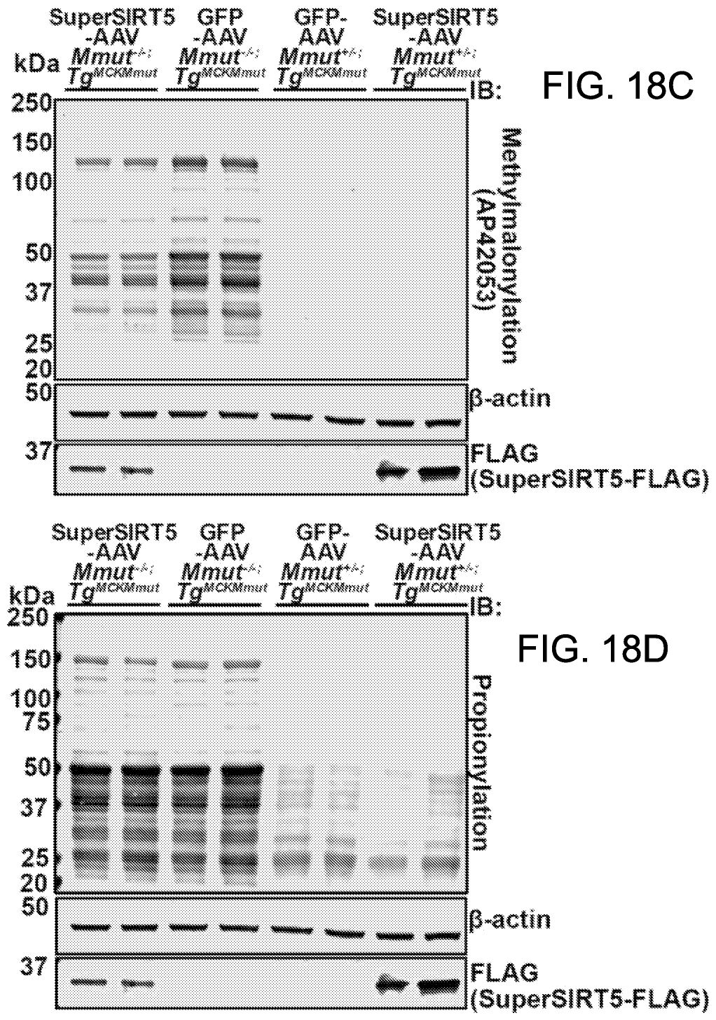

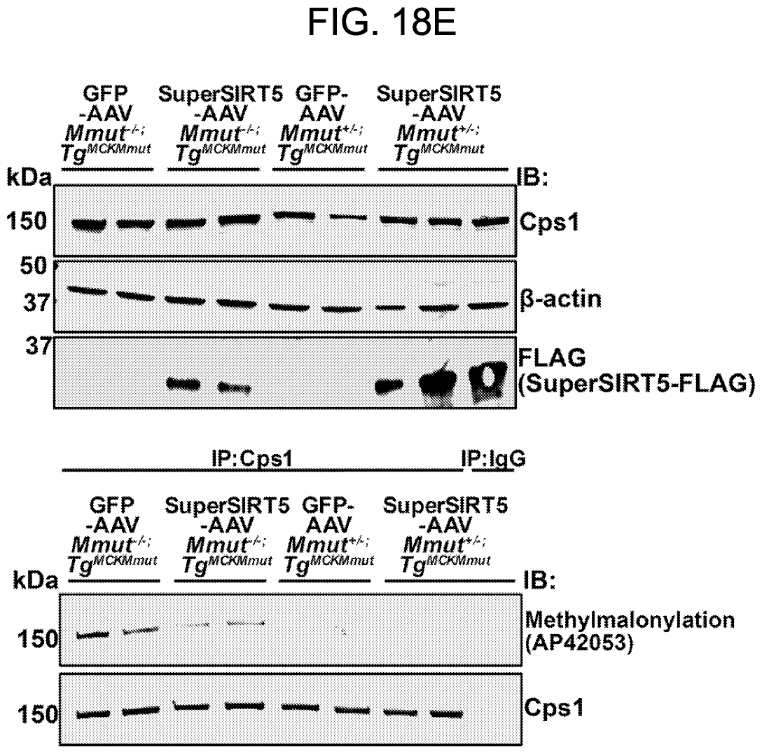

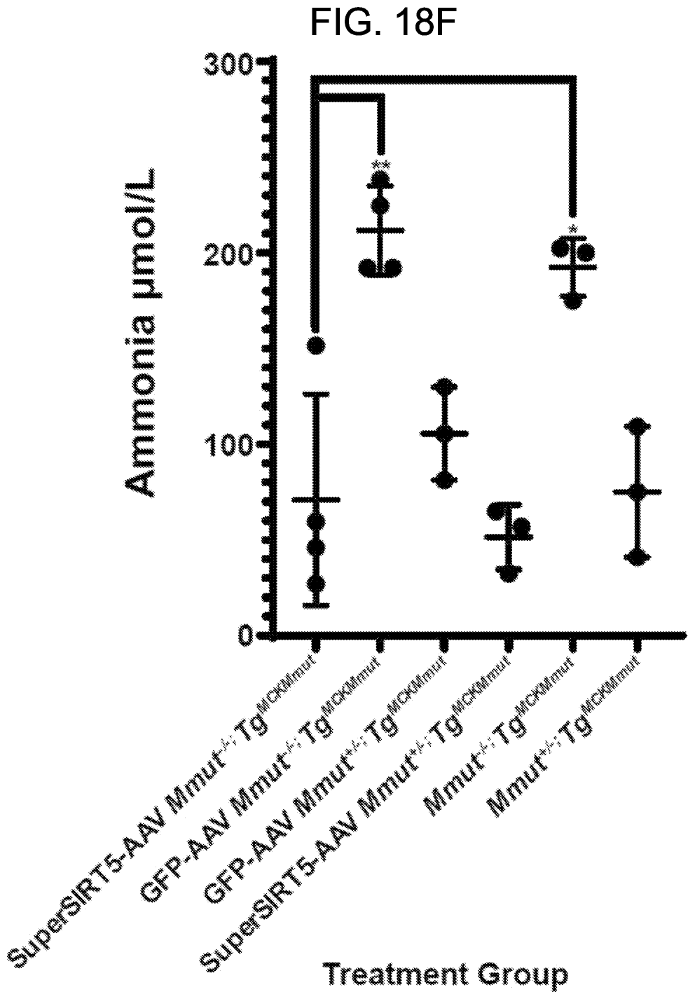

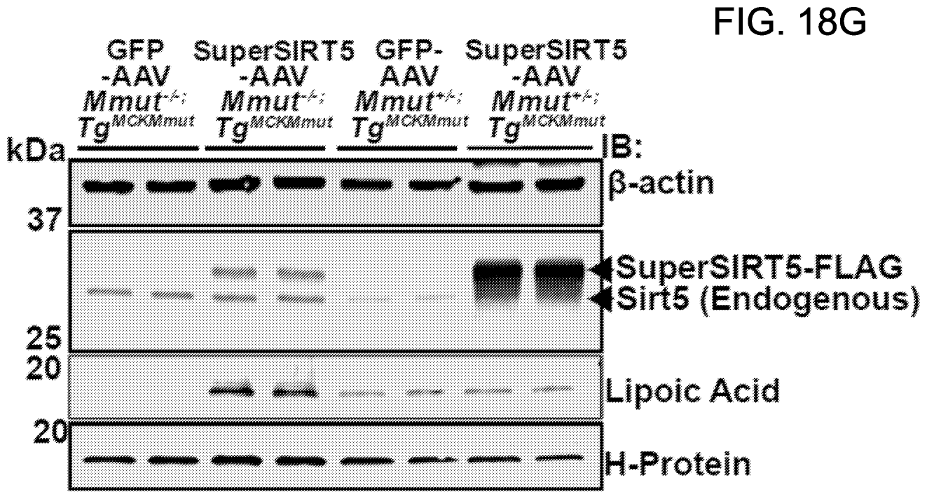

Based on these observations, a mutant SIRT5 having four lysines replaced by arginines was developed, which is resistant to inactivating acylation. Expression of the mutant SIRT5 having four lysines replaced by arginines from an AAV vector in MMA mice, increased body weight, diminished aberrant methylmalonylation, and reduced blood ammonia levels (FIGS. 18A-18G). Thus, the disclosed SIRT5 mutant can be used to treat OAs in vivo, for example using an mRNA-lipid nanoparticle or a viral gene therapy vector, such as AAV, to express the mutant protein. In addition, the new assays provided can be used to identify small molecules that stimulate removal of methylmalonyl- and propionyl-groups.

Provided herein are isolated mutant SIRT5 proteins having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 4 or SEQ ID NO: 10, wherein the mutant SIRT5 protein retains an arginine at one or more of positions 79, 112, 148, and 152 of SEQ ID NO: 4 or SEQ ID NO: 10. In some examples, an isolated mutant SIRT5 protein having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 4 or SEQ ID NO: 10 retains the arginine at all of positions 79, 112, 148, and 152 of SEQ ID NO: 4 or SEQ ID NO: 10. In some examples, an isolated mutant SIRT5 protein comprises or consists of the protein sequence of SEQ ID NO: 4 or SEQ ID NO: 10. In some examples, an isolated mutant SIRT5 protein further includes a purification tag, such as a FLAG-, GST- or Myc-tag (e.g., see SEQ ID NO: 10). Variants of the disclosed isolated mutant SIRT5 proteins, which retain an arginine at one or more of positions 79, 112, 148, and 152 of SEQ ID NO: 4 or SEQ ID NO: 10 (such as retains arginine at all four of positions 79, 112, 148, and 152 of SEQ ID NO: 4 or SEQ ID NO: 10), can be further modified, for example to include one or more additional amino acid substitutions, deletions, and/or additions (such as 1 to 30 of such modifications, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30), such as one or more conservative substitutions (such as 1 to 30 of such substitutions, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30), but retain deacylase activity (e.g., ability to remove malonylation from CPS1) when hyperacylated.

Also provided are isolated nucleic acid molecules that encode a disclosed mutant SIRTS protein. Such nucleic acid molecules can be DNA or RNA, such as mRNA and cDNA. In some examples, the isolated nucleic acid molecule has at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 3 or SEQ ID NO: 9, and encodes an arginine at one or more of positions 79, 112, 148, and 152 of SEQ ID NO: 4 or SEQ ID NO: 10, such as encodes an arginine at all of positions 79, 112, 148, and 152 of SEQ ID NO: 4 or SEQ ID NO: 10. The disclosed mutant SIRTS protein coding sequences can be operably linked to a promoter, such as a constitutive or activatable promoter. In some examples, the promoter is a tissue-specific promoter or organelle-specific promoter, such as a mitochondrial-specific promoter (e.g., the light-strand promoter (LSP) or the heavy-strand promoter (HSP) of the mitochondrial genome). Also provided are mutant SIRTS protein coding sequences (e.g., mRNA) that further include a lipid nanoparticle (LNP). Also provided are vectors that include a mutant SIRTS protein coding sequence, such as a plasmid or viral vector (e.g., adeno-associated virus (AAV) or lentivirus). Also provided are host cells that include such vectors or a mutant SIRTS protein coding sequence (for example as part of a lipid nanoparticle (LNP)), such as a bacterium, mammalian cell (e.g., human cell, such as a cell of a subject with OA, such as MMA or PA) or yeast cell.

Also provided are compositions that include one or more of the disclosed mutant SIRTS proteins, or one or more nucleic acid molecules encoding the one or more of the disclosed mutant SIRTS proteins (such as a vector or plasmid). The composition can further include a pharmaceutically acceptable carrier, such as water or saline. The disclosed compositions can further include one or more other therapeutic agents, such as those used to treat an OA, such as MMA or PA. In one example, a composition further includes one or more of L-carnitine, hydroxycobalamin, vitamin B12, an antibiotic, sodium benzoate, N-carbamylglutamate or combinations thereof. In one example, a composition further includes one or more of a MMUT, MMAA, MMAB, MMACHC, MMACHD, LMBRD1, MCEE, PCCA or PCCB protein or nucleic acid molecule encoding a MMUT, MMAA, MMAB, MMACHC, MMACHD, LMBRD1, MCEE, PCCA or PCCB protein (for example to provide enzyme replacement therapy). In one example, a composition includes one or more nucleic acid molecules encoding a disclosed mutant SIRTS protein (such as an mRNA, vector or plasmid), and a LNP. In some examples, a composition is liquid. In some examples, a composition is frozen. In some examples, a composition is lyophilized The disclosed compositions can be present in a vessel, such as a glass or plastic container, such as a syringe.

The disclosure also provides methods of diagnosis and treatment. For example, provided are methods of diagnosing an OA, such as MMA, IVA, GA1, or PA, a vitamin deficiency, such as vitamin B12 deficiency, or a metabolic disorder, such as a disorder in which the metabolism of vitamin B12 is impaired, such as deficiency of MMACHC (cb1C), MMADHC (cb1D), or LMBDR1 (cb1F). The method can include detecting or measuring one or more (such as 1, 2, 3, 4, 5, 6, 7, 8, 9 or 10) hyperacylated proteins (such as one or more of CPS1, GCSH, SIRT5, TFAM, OPA1, a protein listed in FIG. 4, or a protein belonging to any one of the pathways shown in FIG. 5A and/or FIG. 5B) in a sample (such as blood or a fraction thereof, or a liver sample) from a subject having or suspected of having an OA, vitamin deficiency, or metabolic disorder, wherein detecting one or more hyperacylated proteins diagnoses the subject as having an OA, vitamin deficiency, or metabolic disorder. Provided is a method of diagnosing an OA (such as MMA, IVA, GA1, or

PA), vitamin deficiency or metabolic disorder, by detecting one or more proteins posttranslationally modified with methylmalonyllysine in a sample from a subject having or suspected of having an OA, vitamin deficiency or metabolic disorder. Detecting one or more proteins posttranslationally modified with methylmalonyllysine diagnoses the subject with an OA (such as MMA, IVA, GA1, or PA), vitamin deficiency or metabolic disorder. Also provided is a method of monitoring an OA, vitamin deficiency or metabolic disorder by detecting one or more proteins posttranslationally modified with methylmalonyllation in a sample from the subject having OA, vitamin deficiency or metabolic disorder, wherein the subject having the OA previously received a liver and/or kidney transplant. In some embodiments, the OA is MMA or PA. In some examples of these methods, the one or more proteins are selected from the group consisting of CPS1, GCSH, SIRT5, TFAM, OPAL. In other examples of these methods, the one or more proteins (such as 1, 2, 3, 4, 5, 6, 7, 8, 9 or 10 proteins) are selected from the group consisting of Cps1, Aass, Atxn2, Cttn, F8, Hmgcl, Lrrn3, Nepro, Plin4, Rbm15, Tmem143, Argl, Cct5, Dip2b, Fat2, Harsl, Klrblf, No18, Ptprv, Slclal, Tkfc, Gstm7, Acaa2, Bclaf3, Cwc27, Fam184b, Hmgcs2, Mapls, Nipbl, Plxndl, Rbm27, Topors, Asap3, Cgn, Dnahl, Fgf8, Haus7, Lactb, Nsd3, Rasgeflb, Slc25a1, Tpp2, Hars, Acad8, Bdpl, Cyfip2, Fgr, Hnrnpc, Map2k6, Nipsnapl, Polq, Rev31, Trdn, Adhl, Asl, Chat, Dnah5, Fkbp5, Hibadh, Lyar, PHF20, Rdx, Slc25a5, Tsks, Mdgal, Acinl, C9, Depdc5, Fmrl, Hp, Mapkl, Nodl, Ppplr10, Rida, Trim21, Adnp, Assl, Cisd1, Dockl, Foxc2, Hmcnl, Macfl, Palm, Recq15, Slit3, Ttbk2, Rp1, Aco2, Ccdc40, Dhrsl, Gca, Hpfl, Mb12, Nono, Ppplrl2a, Rprdla, Aebpl, Atp5f1b, Clipl, Dock8, Gapdh, Hs3st3a1, Mcurl, Pcnx3, Rgs3, Sodl, Ttc28, Acsf2, Ccdc90b, Dhx9, Gldn, Hydin, Mctpl, Nudt13, Prcp, Rrsl, Ttn, Aifml, Atp5po, Cmya5, Dock9, Gcc2, Hsf3, Mett117, Pcskl, Sosl, Ushbpl, Adgrbl, Ccdc91, Dip2a, Gludl, Idh2, Mdh2, Nup50, Prdx5, Scnla, Tut7, Akap12, Atr, Col20a1, Dpp6, Gcic, Ids, Mett13, Pdelb, Rmdn3, Sptanl, Vdac3, Adsl, Cdk15, Dlst, Glyat, Igfn1, Mix23, Obscn, Prkcsh, Usp36, Akr1c6, Atxn713, Co124a1, Dym, Ift81, Mgstl, Pde4dip, Robol, Stab2, Vps13b, Agxt, Cep170, Dmgdh, Gott, Il4i1, Mmell, Optn, Prkdc, Slc7a3, Yeats2, Aldhla3, Bhlhe41, Eeal, Gpd2, Il10rb, Morc3, Pdia3, Rrbpl, Stk36, Vps25, Ankefl, Chmplbl, Dnajc14, Grk2, Inhba, Mmp13, Pask, Prr5, Smcla, Znf106, Aldhlll, Blnk, Crisp2, Eeflal, Gpxl, Ildr2, Ms13, Pdzkl, Rtcb, Svil, Zbtb49, Asx11, Cit, Dst, Gtf2e1, Inpp5e, Mmrnl, Pc, Psmb2, Smc4, Znf770, Aldob, Bpifb6, Ctnna3, Efhb, Gstal, Isyl, Mug2, Pgkl, Rubcnl, Tbx2, Zc3h3, Atg14, Claspl, Dyncllil, Hadh, Kiaa1109, Mnl, Pclo, Ptchd4, Snrk, Acatl, Almsl, Bsdcl, Cyp2c37, Elp4, Gstml, Itsn2, Myhl, Pletl, Sec31a, Tcf20, Zfp28, Atmin, Coll lag, Echl, Hadha, Lcp2, Mycbp, Pdia2, Rabepl, Sod2, Cs, Ankrd23, Byes, Cyp2u1, Em16, Gstpl, Kcnk2, Ndufafl, Sec63, Tedc2, Znf518a, Atp5pb, Col4al, Ecil, Hba, Lefl, Mycbp2, Piddl, Raetlb, Tbrgl, Ccdc58, Ankrd34b, Clq13, Cyp3al1, Eri2, Gstzl, Kdm2b, Nebl, Polr2h, Sez6, Tent2, Cyp2c50, Eppkl, Hibch, Lgr4, Naip5, Pla2g4c, Rapgef5, Tent4b, Certl, Anxa6, Ca3, Dbi, Fabpl, Gtpbpl, Kiflc, Nemf, Polrmt, Shc2, Tfap2a, Gsta3, Atp8b5, Ctdspl, Etfa, Hivepl, Lnpk, Nav3, Plaa, Rbbp6, Tlx2, Apexl, Didol, Fam189a1, Hadhb, Kif5b, Nfrkb, Pter, Skt, Tgfbr3, and Gstm2 (see FIG. 4). In other examples, the one or more proteins are selected from any of the proteins belonging to the protein pathways shown in FIGS. 5A-5B.

Further provided is a method of treating an OA (such as MMA, IVA, GA1, or PA), vitamin deficiency or metabolic disorder by detecting one or more proteins posttranslationally modified with methylmalonyllation in a sample from a subject having or suspected of having an OA, vitamin deficiency or metabolic disorder and administering a therapeutically effective amount of an isolated mutant SIRT5 protein, mutant SIRT5 protein coding sequence (such as an mRNA, other nucleic acid, or viral vector expressing such a coding sequence), or a composition comprising such molecules, to a subject having the

OA, vitamin deficiency or metabolic disorder, thereby treating the OA, vitamin deficiency or metabolic disorder. In some embodiments, the OA is MMA or PA. In some examples of this method, the one or more proteins are selected from the group consisting of CPS1, GCSH, SIRT5, TFAM, OPAL In other examples of this method, the one or more proteins (such as 1, 2, 3, 4, 5, 6, 7, 8, 9 or 10 proteins) are selected from the group consisting of Cpsl, Aass, Atxn2, Cttn, F8, Hmgcl, Lrrn3, Nepro, Plin4, Rbm15, Tmem143, Argl, Cct5, Dip2b, Fat2, Harsl, Klrblf, No18, Ptprv, Slclal, Tkfc, Gstm7, Acaa2, Bclaf3, Cwc27, Fam184b, Hmgcs2, Mapls, Nipbl, Plxndl, Rbm27, Topors, Asap3, Cgn, Dnahl, Fgf8, Haus7, Lactb, Nsd3, Rasgeflb, Slc25a1, Tpp2, Hars, Acad8, Bdpl, Cyfip2, Fgr, Hnrnpc, Map2k6, Nipsnapl, Polq, Rev31, Trdn, Adhl, Asl, Chat, Dnah5, Fkbp5, Hibadh, Lyar, PHF20, Rdx, Slc25a5, Tsks, Mdgal, Acinl, C9, Depdc5, Fmrl, Hp, Mapkl, Nodl, Ppplr10, Rida, Trim21, Adnp, Assl, Cisdl, Dockl, Foxc2, Hmcnl, Macfl, Palm, Recq15, S1it3, Ttbk2, Rp1, Aco2, Ccdc40, Dhrsl, Gca, Hpfl, Mb12, Nono, Ppplrl2a, Rprdla, Aebpl, Atp5f1b, Clipl, Dock8, Gapdh, Hs3st3a1, Mcurl, Pcnx3, Rgs3, Sodl, Ttc28, Acsf2, Ccdc90b, Dhx9, Gldn, Hydin, Mctpl, Nudt13, Prcp, Rrsl, Ttn, Aifml, Atp5po, Cmya5, Dock9, Gcc2, Hsf3, Mett117, Pcskl, Sosl, Ushbpl, Adgrbl, Ccdc91, Dip2a, Gludl, Idh2, Mdh2, Nup50, Prdx5, Scnla, Tut7, Akap12, Atr, Co120a1, Dpp6, Gcic, Ids, Mett13, Pdelb, Rmdn3, Sptanl, Vdac3, Adsl, Cdk15, Dlst, Glyat, Igfn1, Mix23, Obscn, Prkcsh, Usp36, Akr1c6, Atxn713, Co124a1, Dym, Ift81, Mgstl, Pde4dip, Robot, Stab2, Vps13b, Agxt, Cep170, Dmgdh, Gott, Il4i1, Mmell, Optn, Prkdc, S1c7a3, Yeats2, Aldhla3, Bhlhe41, Eeal, Gpd2, Il10rb, Morc3, Pdia3, Rrbpl, Stk36, Vps25, Ankefl, Chmplbl, Dnajc14, Grk2, Inhba, Mmp13, Pask, Prr5, Smcla, Znf106, Aldh111, Blnk, Crisp2, Eeflal, Gpxl, Ildr2, Ms13, Pdzkl, Rtcb, Svil, Zbtb49, Asx11, Cit, Dst, Gtf2e1, Inpp5e, Mmrnl, Pc, Psmb2, Smc4, Znf770, Aldob, Bpifb6, Ctnna3, Efhb, Gstal, Isyl, Mug2, Pgkl, Rubcnl, Tbx2, Zc3h3, Atg14, Claspl, Dynellil, Hadh, Kiaa1109, Mn1, Pclo, Ptchd4, Snrk, Acatl, Almsl, Bsdcl, Cyp2c37, Elp4, Gstml, Itsn2, Myhl, Pletl, Sec31a, Tcf20, Zfp28, Atmin, Coll1a2, Echl, Hadha, Lcp2, Mycbp, Pdia2, Rabepl, Sod2, Cs, Ankrd23, Byes, Cyp2u1, Em16, Gstpl, Kcnk2, Ndufafl, Sec63, Tedc2, Znf518a, Atp5pb, Col4al, Ecil, Hba, Left, Mycbp2, Piddl, Raetlb, Tbrgl, Ccdc58, Ankrd34b, Clq13, Cyp3all, Eri2, Gstzl, Kdm2b, Nebl, Polr2h, Sez6, Tent2, Cyp2c50, Eppkl, Hibch, Lgr4, Naip5, Pla2g4c, Rapgef5, Tent4b, Certl, Anxa6, Ca3, Dbi, Fabpl, Gtpbpl, Kiflc, Nemf, Polrmt, Shc2, Tfap2a, Gsta3, Atp8b5, Ctdspl, Etfa, Hivepl, Lnpk, Nav3, Plaa, Rbbp6, Tlx2, Apexl, Didol, Fam189a1, Hadhb, Kif5b, Nfrkb, Pter, Skt, Tgfbr3, and Gstm2 (see FIG. 4). In other examples, the one or more protein are selected from any of the proteins belonging to the protein pathways shown in FIGS. 5A-5B.

Also provided are methods of treating an OA, vitamin deficiency or metabolic disorder that include administering a therapeutically effective amount of an isolated mutant SIRT5 protein, mutant SIRT5 protein coding sequence (such as an mRNA, other nucleic acid, or viral vector expressing such a coding sequence), or a composition comprising such molecules, to a subject having an OA, vitamin deficiency or metabolic disorder, thereby treating the OA, vitamin deficiency or metabolic disorder. In some examples, the nucleic acid administered is part of a LNP. In some examples, the methods of treating an OA, vitamin deficiency or metabolic disorder further include detecting one or more hyperacylated proteins modified with methylmalonylation in a sample from a subject having or suspected of having an OA, vitamin deficiency or metabolic disorder. The methods also include monitoring an OA, vitamin deficiency or metabolic disorder in a subject, by detecting one or more hyperacylated proteins in a sample from the subject having the OA, vitamin deficiency or metabolic disorder, wherein the subject having the OA, vitamin deficiency or metabolic disorder previously received a liver and/or kidney transplant. In some examples, the one or more proteins (such as 1, 2, 3, 4, 5, 6, 7, 8, 9 or 10 proteins) are selected from CPS1, GCSH, SIRT5, TFAM,

OPA1, any protein listed in FIG. 4, and any protein belonging to any one of the protein pathways shown in FIG. 5A and/or FIG. 5B. In some examples, the detecting step includes contacting the sample with an anti-methylmalonyllysine specific antibody or detecting the one or more proteins using mass spectrometry. In some examples, the sample analyzed is a blood sample, plasma sample, urine sample, or liver biopsy sample.

Also provided are methods of reducing post-translational modifications (PTMs) (such as methylmalonylation, malonylation, and/or propionylation) of proteins (such as CPS1 or GCSH) in a subject having an OA, such as MMA or PA. The method can include administering a therapeutically effective amount of an isolated mutant SIRT5 protein, mutant SIRT5 protein coding sequence (such as a vector expressing such as coding sequence), or a composition comprising such molecules, to a subject having OA, thereby reducing PTMs of proteins in the subject having OA.

The disclosed methods can further include administering to the subject having OA a therapeutically effective amount of a MMUT, MMAA, MMAB, MMACHC, MMACHD, LMBRD1, or MCEE enzyme, nucleic acid encoding the enzyme, such as a vector encoding the enzyme (e.g., enzyme replacement therapy); a low-protein high calorie diet; a diet that avoids isoleucine, valine, threonine, and methionine; L-carnitine; hydroxycobalamin; vitamin B12; one or more antibiotics; sodium benzoate; N-carbamylglutamate; or combinations thereof.

Also provided are kits. In some examples, the kit includes an isolated SIRT5 protein or nucleic acid molecule encoding a SIRT5 protein (such as recombinant wildtype (WT) SIRT5 or the mutant SIRT5 K4R), ultra-pure, non-acylated BSA and ultra-pure acylated BSA, nicotinamide (NAM); nicotinamide adenine dinucleotide (NAD+); and/or an anti-acyllysine antibody (such as an antibody, for example a polyclonal antibody, that specifically binds to malonyllysine and methylmalonyllysine). In some examples, the kit further includes a liver extract, for example from a mammal, such as from a mammal with an OA, from a mammal without an OA, or extracts from both. In some examples, the mammal is a human, non-human primate, rat, rabbit, or mouse.

### Mutant SIRT5 Proteins

The present disclosure provides isolated mutant SIRT5 proteins that can include an arginine instead of a lysine at one or more of positions 79, 112, 148, and 152 of SEQ ID NO: 4, such as 1, 2, 3, or all four positions (e.g., a SIRT5 mutant having K79R, K112R, K148R, and K152R substitutions, wherein the numbering is relative to a native SIRT5 human sequence, such as SEQ ID NO: 4). The mutant SIRT5 protein can include one or more additional point mutations (such as amino acid substitutions, deletions, additions, or combinations thereof). In addition, a mutant SIRT5 protein can include a tag or linker, such as a purification tag at its N- or C-terminus (e.g., FLAG tag, HIS tag, Myc tag, see e.g., SEQ ID NO: 10). In some examples, the mutant SIRT5 protein includes an immunoglobulin Fc domain, such as a human Fc protein, such as the human IgG1 Fc (e.g., Czajkowsky et al., EMBO Mol. Med. 4:1015-28, 2012, herein incorporated by reference) at either N-terminal or C-terminal end, for example to enhance stability of the protein and therefore serum half-life, and/or can be used to as a means to purify the mutant SIRT5 protein on protein A or Protein G sepharose beads. Such proteins and corresponding coding sequences can be used in the methods provided herein. In some examples, the mutant SIRT5 protein includes cell penetrating peptide. The cell penetrating peptide can be at the N- or C-terminus of the mutant SIRT5 protein. Cell penetrating peptides are usually short peptides (40 amino acids or less) that are highly cationic and usually rich in arginine and lysine that can facilitate cellular intake/uptake of proteins. Exemplary cell penetrating peptides that can be used include hydrophilic peptides (e.g., TAT [YGRKKRRQRRR; SEQ ID NO: 22], SynB1 [RGGRLSYSRRRFSTSTGR; SEQ ID NO: 23], SynB3 [RRLSYSRRRF; SEQ ID NO: 24], PTD-4 [PIRRRKKLRRLK; SEQ ID NO: 25], PTD-5 [RRQRRTSKLMKR; SEQ ID NO: 26], FHV Coat-(35-49) [RRRRNRTRRNRRRVR; SEQ ID NO: 27], BMV Gag-(7-25) [KMTRAQRRAAARRNRWTAR; SEQ ID NO: 28], HTLV-II Rex-(4-16) [TRRQRTRRARRNR; SEQ ID NO: 29], D-Tat [GRKKRRQRRRPPQ; SEQ ID NO: 30], R9-Tat GRRRRRRRRRPPQ [SEQ ID NO: 31] and penetratin [RQIKWFQNRRMKWKK; SEQ ID NO: 32]), amphiphilic peptides (e.g., MAP [KLALKLALKLALALKLA; SEQ ID NO: 33], SBP [MGLGLHLLVLAAALQGAWSQPKKKRKV; SEQ ID NO: 34], FBP [GALFLGWLGAAGSTMGAWSQPKKKRKV; SEQ ID NO: 35], MPG ac-[GALFLGFLGAAGSTMGAWSQPKKKRKV-cya; SEQ ID NO: 36], MPG(ANLS) [ac-GALFLGFLGAAGSTMGAWSQPKSKRKV-cya; SEQ ID NO: 37], Pep-2 [ac-KETWFETWFTEWSQPKKKRKV-cya; SEQ ID NO: 38], and transportan [GWTLNSAGYLLGKINLKALAALAKKIL; SEQ ID NO: 39]), periodic sequences (e.g., pVec, polyarginines R×N (4<N<17) chimera, polylysines KxN (4<N<17) chimera, (RAca)6R, (RAbu)6R, (RG)6R, (RM)6R, (RT)6R, (RS)6R, R10, (RA)6R, R7, and pep-1 [ac-KETWWETWWTEWSQPKKKRKV-cya; SEQ ID NO: 40]), Cr10 (a cyclic pol-arginine CPP), TAT₄₈₋₅₇, TAT₄₇₋₅₇, or TAT₄₉₋₅₇; penetratin; Pep-1; substance P, SP; polyarginines, such as R5-R12; pVEC; transportan; MAP; diatos peptide vector 1047, DPV1047, VECTOCELL®; MPG; ADP ribosylation factor, ARF, such as ARF₁₋₂₂; BPrPr (such as BPrPr_{i—} 28); p28; VT5; Bac 7, such as Baci_24; C105Y; PFVYLI (SEQ ID NO: 41); and Pep-7. Mutant SIRT5 proteins can include an N-terminal cap such as formyl, acetyl, 2-18 carbons acyls, arylacyl (like benzoyl), heteroarylacyl (like 2-acetylpyridine), carbamates (like t-butylcarbamate), succinyl, alkyl or arylsulfonamide and/or a C-terminal cap, such as amide, acid, aldehyde, and esters (aryl, alkyl, heteroaryl, heteroalkyl like polyethylene glycols of 2-20 repeating units).

In some examples, the disclosed SIRT5 mutant proteins have similar, the same or improved de-acylating activity compared to mature native SIRT5 (e.g., SEQ ID NO: 2), such as no less than 80% of the deacylating activity of a native SIRT5 protein, no less than 90%, no less than 95%, no less than 98%, no less than 99%, at least 100%, at least 110% or at least 120% of the de-acylating activity of a native SIRT5 protein. In some examples, the disclosed SIRT5 mutant proteins have increased resistance to acylation inactivation, such as an increase of at least 20%, at least 30%, at least 40%, at least 50%, at least 75%, at least 80%, at least 90%, at least 95%, at least 98%, at least 99%, or at least 100%. For example, a mutant SIRT5 protein can have both the same or improved de-acylating activity of a native SIRT5 protein and increased resistance to acylation inactivation compared to a native SIRT5 protein without the modification. Methods of measuring de-acylating activity and acylation inactivation are provided herein. In some examples, the disclosed SIRT5 mutant proteins have similar, the same or increased reduction of aberrant methylmalonylation activity compared to mature native SIRT5 (e.g., SEQ ID NO: 2), such as no less than 80% of the ability to decrease methylmalonylation activity of a native SIRT5 protein, no less than 90%, no less than 95%, no less than 98%, no less than 99%, at least 100%, at least 110% or at least 120% of the ability to decrease methylmalonylation activity of a native SIRT5 protein. In some examples, the disclosed SIRT5 mutant proteins have similar, the same, or increased ability to reduce blood ammonia levels in an OA subject (such as a subject with MMA) compared to mature native SIRT5 (e.g., SEQ ID NO: 2), such as no less than 80% of the ability to reduce blood ammonia levels in an OA subject of a native SIRT5 protein, no less than 90%, no less than 95%, no less than 98%, no less than 99%, at least 100%, at least 110% or at least 120% of the ability of a native SIRT5 protein to reduce blood ammonia levels in an OA subject.

In some examples, a mutant SIRT5 protein includes at least 70 consecutive amino acids of SEQ ID NO: 4 or SEQ ID NO: 10, and has K79R, K112R, K148R, and K152R substitutions. Thus, in some examples, a mutant SIRT5 protein includes at least 71, at least 72, at least 73, at least 74, at least 75, at least 100, at least 125, at least 150, at least 200, at least 225, at least 250, at least 275, or at least 300 consecutive amino acid of SEQ ID NO: 4.

In some examples, a mutant SIRT5 protein is at least 70 amino acids in length, such as at least 100, at least 125, at least 130, at least 135, at least 140, at least 145, at least 150, at least 155, at least 160, at least 175, at least 200, at least 225, at least 250, at least 275, or at least 300 amino acids in length, such as 70-400, 125-350, 200-350, 250-350, 275-325, 300-400, 300-350, or 300-325 amino acids in length.

In some examples, a mutant SIRT5 protein includes an arginine instead of a lysine at one or more of positions 79, 112, 148, and 152 of SEQ ID NO: 4 or SEQ ID NO: 10, such as 1, 2, 3, or all four positions (e.g., a SIRT5 mutant having K79R, K112R, K148R, and K152R substitutions, wherein the numbering is relative to a native SIRT5 human sequence, such as SEQ ID NO: 2), and further includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35 or at least 40 amino acid substitutions (such as conservative amino acid substitutions), such as 1-30, 1-10, 4-8, 5-12, 5-10, 5-25, 10-30, 20-30, 1, 2, 3, 4, 5, 6, 7, 8, 9, 10, 11, 12, 13, 14, or 15 amino acid substitutions (such as conservative amino acid substitutions). Examples of amino acids which may be substituted for an original amino acid in a protein and which are regarded as conservative substitutions include: Ser for Ala; Lys for Arg; Gln or His for Asn; Glu for Asp; Ser for Cys; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val. In some examples, a mutant SIRT5 protein with additional amino acid substitutions retains the same or improved de-acylating activity of a native SIRT5 protein and increased resistance to acylation inactivation compared to a native SIRT5 protein without the modification.

A mutant SIRT5 protein that includes an arginine instead of a lysine at one or more of positions 79, 112, 148, and 152 of SEQ ID NO: 4 or SEQ ID NO: 10, such as 1, 2, 3, or all four positions (e.g., a SIRT5 mutant having K79R, K112R, K148R, and K152R substitutions, wherein the numbering is relative to a native SIRT5 human sequence, such as SEQ ID NO: 2), can further include one or more additional mutations, such as a single insertion, a single deletion, a single substitution, or combinations thereof. In some examples, the mutant SIRT5 protein further includes 1-30 insertions, 1-30 deletions, 1-30 substitutions, or any combination thereof (e.g., single insertion together with 1-29 substitutions). In some examples, the disclosure provides a variant of any disclosed mutant SIRT5 protein having 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 additional amino acid changes (but retains arginine instead of a lysine at one or more of positions 79, 112, 148, and 152 of SEQ ID NO: 4, such as 1, 2, 3, or all four positions (e.g., a SIRT5 mutant having K79R, K112R, K148R, and K152R substitutions)). In some examples, a mutant SIRT5 protein includes an arginine instead of a lysine at one or more of positions 79, 112, 148, and 152 of SEQ ID NO: 4 or SEQ ID NO: 10, such as 1, 2, 3, or all four positions (e.g., a SIRT5 mutant having K79R, K112R, K148R, and K152R substitutions, wherein the numbering is relative to a native SIRT5 human sequence, such as SEQ ID NO: 2), and further includes 1-50 insertions, 1-50 deletions, 1-50 substitutions, or any combination thereof (e.g., 1-20 amino acid deletions together with 1-20 amino acid substitutions). In some examples, the disclosure provides a variant of any of SEQ ID NOS: 4 and 10 having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 additional amino acid changes (but retains the K79R, K112R, K148R, and/or K152R substitutions).

More substantial changes can be made by using substitutions that are less conservative, e.g., selecting residues that differ more significantly in their effect on maintaining: (a) the structure of the polypeptide backbone in the area of the substitution, for example, as a sheet or helical conformation; (b) the charge or hydrophobicity of the polypeptide at the target site; or (c) the bulk of the side chain. The substitutions that in general are expected to produce the greatest changes in polypeptide function are those in which: (a) a hydrophilic residue, e.g., serine or threonine, is substituted for (or by) a hydrophobic residue, e.g., leucine, isoleucine, phenylalanine, valine or alanine; (b) a cysteine or proline is substituted for (or by) any other residue; (c) a residue having an electropositive side chain, e.g., lysine, arginine, or histidine, is substituted for (or by) an electronegative residue, e.g., glutamic acid or aspartic acid; or (d) a residue having a bulky side chain, e.g., phenylalanine, is substituted for (or by) one not having a side chain, e.g., glycine.

The effects of these amino acid substitutions (or other deletions or additions) can be assessed by analyzing the function of a mutant SIRT5 protein, by analyzing the ability of the variant protein to de-acylate a protein (such as CPS1 or GCSH), for example to depropionylate or demethylmalonylate the protein.

Specific exemplary SIRT5 mutant proteins are shown in SEQ ID NOS: 4 and 10 (wherein SEQ ID NO: 10 includes a FLAG tag at the C-terminus). In some examples, a SIRT5 mutant protein includes at least 80% sequence identity to SEQ ID NO: 4 or SEQ ID NO: 10, while retaining the arginine at one or more of positions 79, 112, 148, and 152 of SEQ ID NO: 4 or 10, such as 1, 2, 3, or all four positions (e.g., having K79R, K112R, K148R, and K152R substitutions). Thus, a SIRT5 mutant protein can have at least 85%, at least 90%, at least 95%, at least 96%, at least 97%, at least 98% or at least 99% sequence identity to SEQ ID NO: 4 or SEQ ID NO: 10, and retain the arginine at one or more of positions 79, 112, 148, and 152 of SEQ ID NO: 4 or SEQ ID NO: 10, such as 1, 2, 3, or all four positions (e.g., having K79R, K112R, K148R, and K152R substitutions). In some examples, the SIRT5 mutant protein includes or consists of SEQ ID NO: 4 or SEQ ID NO: 10. The disclosure encompasses variants of the disclosed SIRT5 mutant proteins, such as SEQ ID NO: 4 or SEQ ID NO: 10 having K79R, K112R, K148R, and/or K152R substitutions (in some examples all 4 substitutions), and 1 to 50, 1 to 40, 1 to 30, 1 to 20, 1 to 10, 1 to 5, 5 to 25, 2 to 25, 3 to 30, 5 to 15, or 5 to 10 mutations, such as conservative amino acid substitutions. The mutant SIRT5 proteins can be used to generate a chimeric or fusion protein including the SIRT5 mutant.

For example, variants of a mutant SIRT5 protein shown in SEQ ID NO: 4 or SEQ ID NO: 10 include those having at least 80%, at least 85%, at least 90%, at least 95%, at least 96%, at least 97%, at least 98%, or at least 99% sequence identity to SEQ ID NO: 4 or SEQ ID NO: 10 and retain the arginine at one or more of positions 79, 112, 148, and 152 of SEQ ID NO: 4 or SEQ ID NO: 10, such as 1, 2, 3, or all four positions (e.g., having K79R, K112R, K148R, and K152R substitutions), and retain the ability to de-acylate a protein (such as CPS1 or GCSH), for example to de-propionylate or de-methylmalonyllysine the protein, for example to treat an OA such as MMA or PA in a mammal. Thus, variants of a mutant SIRT5 protein shown in SEQ ID NO: 4 or SEQ ID NO: 10 include those having at least 80%, at least 85%, at least 90%, at least 95%, at least 96%, at least 97%, at least 98%, or at least 99% sequence identity to SEQ ID NO: 4 or SEQ ID NO: 10 and retain the arginine at one or more of positions 79, 112, 148, and 152 of SEQ ID NO: 4 or SEQ ID NO: 10, such as 1, 2, 3, or all four positions (e.g., having K79R, K112R, K148R, and K152R substitutions) are of use in the disclosed methods.

Although mutations to SIRT5 are noted by a particular amino acid, one skilled in the art will appreciate that the corresponding amino acid can be mutated in any SIRT5 sequence. For example, K79 of SEQ ID NO: 4 (human sequence) corresponds to K79 of the mouse SIRT5 sequence.

Generation of Proteins hi one example, variant SIRT5 proteins are produced by manipulating the nucleotide sequence encoding a peptide using procedures such as site-directed mutagenesis PCR. Such variants can also be chemically synthesized.

Isolation and purification of recombinantly expressed mutated SIRT5 proteins can be carried out by conventional means, such as preparative chromatography and immunological separations. Once expressed, mutated SIRT5 proteins can be purified according to standard procedures, including ammonium sulfate precipitation, affinity columns, column chromatography, and the like (see, generally, R. Scopes, Protein Purification, Springer-Verlag, N.Y., 1982). Substantially pure compositions of at least about 90 to 95% homogeneity are disclosed herein, and 98 to 99% or more homogeneity can be used for pharmaceutical purposes.

In addition to recombinant methods, mutated SIRT5 proteins disclosed herein can also be constructed in whole or in part using standard peptide synthesis. In one example, mutated SIRT5 proteins are synthesized by condensation of the amino and carboxyl termini of shorter fragments. Methods of forming peptide bonds by activation of a carboxyl terminal end (such as by the use of the coupling reagent N, N′-dicylohexylcarbodimide) can be used.

Mutated SIRT5 Nucleic Acid Molecules and Vectors Nucleic acid molecules encoding a mutated SIRT5 protein are encompassed by this disclosure. The nucleic acid molecules include DNA and RNA, such as cDNA and mRNA. Based on the genetic code, nucleic acid sequences coding for any mutated SIRT5 sequence, such as a SIRT5 mutant protein having at least 80%, at least 85%, at least 90%, at least 95%, at least 96%, at least 97%, at least 98% or at least 99% sequence identity to SEQ ID NO: 4 or SEQ ID NO: 10, and retaining the arginine at one or more of positions 79, 112, 148, and 152 of SEQ ID NO: 4 or SEQ ID NO: 10, such as 1, 2, 3, or all four positions (e.g., having K79R, K112R, K148R, and K152R substitutions), can be generated. In some examples, the coding sequence is optimized for expression in a host cell, such as a host cell used to express the mutant SIRT5 protein.

In one example, a mutant SIRT5 nucleic acid coding sequence has at least 60%, at least 70%, at least 75%, at least 80%, at least 85%, at least 90%, at least 92%, at least 95%, at least 96%, at least 97%, at least 98%, or at least 99% sequence identity to SEQ ID NO: 3 or SEQ ID NO: 9, can readily be produced using the amino acid sequences provided herein, and the genetic code. In addition, one of skill can readily construct a variety of clones containing functionally equivalent nucleic acids, such as nucleic acids which differ in sequence but which encode the same mutant SIRT5 protein sequence. In one example, a mutant SIRT5 nucleic acid coding sequence comprises or consists of the sequence of SEQ ID NO: 3 or SEQ ID NO: 9.

Nucleic acid molecules include DNA, cDNA and RNA sequences that encode a mutated SIRT5 peptide. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3′ Edition, W.H. 5 Freeman and Co., N.Y.).

Codon preferences and codon usage tables for a particular species can be used to engineer isolated nucleic acid molecules encoding a mutated SIRT5 protein (such as a nucleic acid molecule encoding a protein having at least 80%, at least 85%, at least 90%, at least 95%, at least 96%, at least 97%, at least 98% or at least 99% sequence identity to SEQ ID NO: 4 or SEQ ID NO: 10, and retaining the arginine at one or more of positions 79, 112, 148, and 152 of SEQ ID NO: 4 or SEQ ID NO: 10, such as 1, 2, 3, or all four positions (e.g., having K79R, K112R, K148R, and K152R substitutions)) that take advantage of the codon usage preferences of that particular species.

A nucleic acid encoding a mutant SIRT5 protein (such as one encoding a protein having at least 80%, at least 85%, at least 90%, at least 95%, at least 96%, at least 97%, at least 98% or at least 99% sequence identity to SEQ ID NO: 4 or SEQ ID NO: 10, and retains the arginine at one or more of positions 79, 112, 148, and 152 of SEQ ID NO: 4 or SEQ ID NO: 10, such as 1, 2, 3, or all four positions (e.g., having K79R, K112R, K148R, and K152R substitutions)) can be cloned or amplified by in vitro methods, such as the polymerase chain reaction (PCR), the ligase chain reaction (LCR), the transcription-based amplification system (TAS), the self-sustained sequence replication system (3SR) and the QI3 replicase amplification system (QB). A wide variety of cloning and in vitro amplification methodologies, as well as chemical synthesis methods, can be used. In addition, nucleic acid sequences encoding a mutant SIRT5 protein (such as a nucleic acid sequence encoding a protein having at least 80%, at least 85%, at least 90%, at least 95%, at least 96%, at least 97%, at least 98% or at least 99% sequence identity to SEQ ID NO: 4 or SEQ ID NO: 10, and retaining the arginine at one or more of positions 79, 112, 148, and 152 of SEQ ID NO: 4 or SEQ ID NO: 10, such as 1, 2, 3, or all four positions (e.g., having K79R, K112R, K148R, and K152R substitutions)) can be prepared by cloning techniques. Examples of appropriate cloning and sequencing techniques can be found in Sambrook et al. (ed.), Molecular Cloning: A Laboratory Manual 2nd ed., vol. 1-3, Cold Spring Harbor Laboratory Press, Cold Spring, Harbor, N.Y., 1989, and Ausubel et al., (1987) in “Current Protocols in Molecular Biology,” John Wiley and Sons, New York, N.Y.

Nucleic acid sequences encoding a mutated SIRT5 protein (such as a nucleic acid sequence encoding a protein having at least 80%, at least 85%, at least 90%, at least 95%, at least 96%, at least 97%, at least 98% or at least 99% sequence identity to SEQ ID NO: 4 or SEQ ID NO: 10, and retaining the arginine at one or more of positions 79, 112, 148, and 152 of SEQ ID NO: 4 or SEQ ID NO: 10, such as 1, 2, 3, or all four positions (e.g., having K79R, K112R, K148R, and K152R substitutions)) can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide, which can be converted into double stranded DNA by hybridization with a complementary sequence, or by polymerization with a DNA polymerase using the single strand as a template.

In one example, a mutant SIRTS protein (such as a protein having at least 80%, at least 85%, at least 90%, at least 95%, at least 96%, at least 97%, at least 98% or at least 99% sequence identity to SEQ ID NO: 4 or SEQ ID NO: 10, and retaining the arginine at one or more of positions 79, 112, 148, and 152 of SEQ ID NO: 4 or SEQ ID NO: 10, such as 1, 2, 3, or all four positions (e.g., having K79R, K112R, K148R, and K152R substitutions)) is prepared by inserting a cDNA encoding the mutant SIRTS protein into a vector.

The insertion can be made so that the mutant SIRTS protein is read in frame so that the mutant SIRTS protein is produced.

The mutated SIRTS protein nucleic acid coding sequence (such as a nucleic acid sequence encoding a protein having at least 80%, at least 85%, at least 90%, at least 95%, at least 96%, at least 97%, at least 98% or at least 99% sequence identity to SEQ ID NO: 4 or SEQ ID NO: 10, and retaining the arginine at one or more of positions 79, 112, 148, and 152 of SEQ ID NO: 4 or SEQ ID NO: 10, such as 1, 2, 3, or all four positions (e.g., having K79R, K112R, K148R, and K152R substitutions)) can be inserted into an expression vector including, but not limited to a plasmid, virus or other vehicle that can be manipulated to allow insertion or incorporation of sequences and can be expressed in either prokaryotes or eukaryotes. In one example, a vector comprising the nucleic acid molecule encoding a mutated SIRTS protein has at least 80%, at least 85%, at least 90%, at least 95%, at least 96%, at least 97%, at least 98%, at least 99%, or 100% sequence identity to SEQ ID NO: 11 or SEQ ID NO: 12. Hosts can include microbial, yeast, insect, plant and mammalian cells and organisms. The vector can encode a selectable marker, such as a thymidine kinase gene, or an antibiotic resistance gene. Nucleic acid sequences encoding a mutated SIRTS protein (such as a nucleic acid sequence encoding a protein having at least 80%, at least 85%, at least 90%, at least 95%, at least 96%, at least 97%, at least 98% or at least 99% sequence identity to SEQ ID NO: 4 or SEQ ID NO: 10, and retaining the arginine at one or more of positions 79, 112, 148, and 152 of SEQ ID NO: 4 or SEQ ID NO: 10, such as 1, 2, 3, or all four positions (e.g., having K79R, K112R, K148R, and K152R substitutions)) can be operatively linked to expression control sequences. An expression control sequence operatively linked to a mutated SIRT5 protein coding sequence is ligated such that expression of the mutant SIRT5 protein coding sequence is achieved under conditions compatible with the expression control sequences. The expression control sequences include, but are not limited to appropriate promoters, enhancers, transcription terminators, a start codon (i.e., ATG) in front of a mutated SIRT5 protein-encoding gene, splicing signal for introns, maintenance of the correct reading frame of that gene to permit proper translation of mRNA, and stop codons. In one example the promoter is a constitutive promoter. In one example the promoter is an inducible promoter. In one example the promoter is a tissue- or organelle-specific promoter, such as a liver-specific promoter, or mitochondrial-specific promoter.

In one embodiment, vectors are used for expression in yeast such as S. cerevisiae, P. pastoris, or Kluyveromyces lactis. Several promoters are known to be of use in yeast expression systems such as the constitutive promoter plasma membrane H+-ATPase (PMA1), glyceraldehyde-3-phosphate dehydrogenase (GPD), phosphoglycerate kinase-1 (PGK1), alcohol dehydrogenase-1 (ADH1), and pleiotropic drug-resistant pump (PDR5). In addition, many inducible promoters are of use, such as GAL1-10 (induced by galactose), PHO5 (induced by low extracellular inorganic phosphate), and tandem heat shock HSE elements (induced by temperature elevation to 37° C.). Promoters that direct variable expression in response to a titratable inducer include the methionine-responsive MET3 and MET25 promoters and copper-dependent CUP1 promoters. Any of these promoters may be cloned into multicopy (2μ) or single copy (CEN) plasmids to give an additional level of control in expression level. The plasmids can include nutritional markers (such as URA3, ADE3, HIS1, and others) for selection in yeast and antibiotic resistance (AMP) for propagation in bacteria. Plasmids for expression on K. lactis are known, such as pKLAC1. Thus, in one example, after amplification in bacteria, plasmids can be introduced into the corresponding yeast auxotrophs by methods similar to bacterial transformation. The nucleic acid molecules encoding a mutated SIRTS protein (such as a nucleic acid molecule encoding a protein having at least 80%, at least 85%, at least 90%, at least 95%, at least 96%, at least 97%, at least 98%, or at least 99% sequence identity to SEQ ID NO: 4 or SEQ ID NO: 10, and retaining the arginine at one or more of positions 79, 112, 148, and 152 of SEQ ID NO: 4 or SEQ ID NO: 10, such as 1, 2, 3, or all four positions (e.g., having K79R, K112R, K148R, and K152R substitutions)) can be expressed in insect cells.

A mutated SIRTS protein (such as a protein having at least 80%, at least 85%, at least 90%, at least 95%, at least 96%, at least 97%, at least 98% or at least 99% sequence identity to SEQ ID NO: 4 or SEQ ID NO: 10, and retaining the arginine at one or more of positions 79, 112, 148, and 152 of SEQ ID NO: 4 or SEQ ID NO: 10, such as 1, 2, 3, or all four positions (e.g., having K79R, K112R, K148R, and K152R substitutions)) can be expressed in a variety of yeast strains. For example, seven pleiotropic drug-resistant transporters, YOR1, SNQ2, PDR5, YCF1, PDR10, PDR11, and PDR15, together with their activating transcription factors, PDR1 and PDR3, have been simultaneously deleted in yeast host cells, rendering the resultant strain sensitive to drugs. Yeast strains with altered lipid composition of the plasma membrane, such as the erg6 mutant defective in ergosterol biosynthesis, can also be utilized. Proteins that are highly sensitive to proteolysis can be expressed in a yeast cell lacking the master vacuolar endopeptidase Pep4, which controls the activation of other vacuolar hydrolases. Heterologous expression in strains carrying temperature-sensitive (ts) alleles of genes can be employed if the corresponding null mutant is inviable.

Viral vectors, such as an AAV vector, that encode a mutated SIRT5 protein (such as one encoding a protein having at least 80%, at least 85%, at least 90%, at least 95%, at least 96%, at least 97%, at least 98% or at least 99% sequence identity to SEQ ID NO: 4 or SEQ ID NO: 10, and retaining the arginine at one or more of positions 79, 112, 148, and 152 of SEQ ID NO: 4 or SEQ ID NO: 10, such as 1, 2, 3, or all four positions (e.g., having K79R, K112R, K148R, and K152R substitutions)) are provided (e.g., see SEQ ID

NOS: 11 and 12, which provide AAV vectors containing a K4R mutant SIRT5 protein coding sequence). In one example, a viral vector encoding a mutated SIRT5 protein has at least 80%, at least 85%, at least 90%, at least 95%, at least 96%, at least 97%, at least 98% or at least 99% sequence identity to SEQ ID NO: 11 or SEQ ID NO: 12, wherein the encoded mutant SIRTS includes an arginine at one or more of positions 79, 112, 148, and 152 of SEQ ID NO: 4 or 10, such as 1, 2, 3, or all four positions (e.g., encodes K79R, K112R,

K148R, and K152R substitutions). In some examples, such a vector is expressed in the liver.

Exemplary viral vectors include adeno-associated virus (AAV), polyoma, SV40, adenovirus, vaccinia virus, herpes viruses including HSV and EBV, Sindbis viruses, alphaviruses and retroviruses of avian, murine, and human origin. Baculovirus (Autographa californica multinuclear polyhedrosis virus; AcMNPV) vectors are also known in the art, and may be obtained from commercial sources. Other suitable vectors include retrovirus vectors, orthopox vectors, avipox vectors, fowlpox vectors, capripox vectors, suipox vectors, adenoviral vectors, herpes virus vectors, alpha virus vectors, baculovirus vectors, Sindbis virus vectors, vaccinia virus vectors and poliovirus vectors. Specific exemplary vectors are poxvirus vectors such as vaccinia virus, fowlpox virus and a highly attenuated vaccinia virus (MVA), adenovirus, baculovirus and the like. Pox viruses of use include orthopox, suipox, avipox, and capripox virus. Orthopox include vaccinia, ectromelia, and raccoon pox. One example of an orthopox of use is vaccinia. Avipox includes fowlpox, canary pox and pigeon pox. Capripox include goatpox and sheeppox. In one example, the suipox is swinepox. Other viral vectors that can be used include other DNA viruses such as herpes virus, adeno-associated virus, and adenoviruses, and RNA viruses such as retroviruses and polio. Viral vectors that encode a mutated SIRTS protein (such as a viral vector encoding a protein having at least 80%, at least 85%, at least 90%, at least 95%, at least 96%, at least 97%, at least 98% or at least 99% sequence identity to SEQ ID NO: 4 or SEQ ID NO: 10, and retaining the arginine at one or more of positions 79, 112, 148, and 152 of SEQ ID NO: 4 or SEQ ID NO: 10, such as 1, 2, 3, or all four positions (e.g., having K79R, K112R, K148R, and K152R substitutions)) can include at least one expression control element operationally linked to the nucleic acid sequence encoding the mutated SIRTS protein. The expression control elements are inserted in the vector to control and regulate the expression of the nucleic acid sequence. Examples of expression control elements of use in these vectors include, but are not limited to, lac system, operator and promoter regions of phage lambda, yeast promoters and promoters derived from polyoma, adenovirus, retrovirus or SV40. Additional operational elements include, but are not limited to, leader sequence, termination codons, polyadenylation signals and any other sequences necessary for the appropriate transcription and subsequent translation of the nucleic acid sequence encoding the mutated SIRT5 protein in the host system. The expression vector can contain additional elements necessary for the transfer and subsequent replication of the expression vector containing the nucleic acid sequence in the host system. Examples of such elements include, but are not limited to, origins of replication and selectable markers. Such vectors can be constructed using methods provided in Ausubel et al., (1987) in “Current Protocols in Molecular Biology,” John Wiley and Sons, New York, N.Y., and are commercially available. Basic techniques for preparing recombinant DNA viruses containing a heterologous DNA sequence encoding the mutated SIRT5 protein (such as one encoding a protein having at least 80%, at least 85%, at least 90%, at least 95%, at least 96%, at least 97%, at least 98%, or at least 99% sequence identity to SEQ ID NO: 4 or SEQ ID NO: 10, and retaining the arginine at one or more of positions 79, 112, 148, and 152 of SEQ ID NO: 4 or SEQ ID NO: 10, such as 1, 2, 3, or all four positions (e.g., having K79R, K112R, K148R, and K152R substitutions)) can be used. Such techniques involve, for example, homologous recombination between the viral DNA sequences flanking the DNA sequence in a donor plasmid and homologous sequences present in the parental virus. The vector can be constructed for example by steps known in the art, such as by using a unique restriction endonuclease site that is naturally present or artificially inserted in the parental viral vector to insert the heterologous DNA.

When the host is a eukaryote, such methods of transfection of DNA as calcium phosphate coprecipitates, conventional mechanical procedures such as microinjection, electroporation, insertion of a plasmid encased in liposomes, or virus vectors can be used. Eukaryotic cells can also be co-transformed with polynucleotide sequences encoding a mutated SIRT5 protein (such as a polynucleotide sequence encoding a protein having at least 80%, at least 85%, at least 90%, at least 95%, at least 96%, at least 97%, at least 98%, or at least 99% sequence identity to SEQ ID NO: 4 or SEQ ID NO: 10, and retaining the arginine at one or more of positions 79, 112, 148, and 152 of SEQ ID NO: 4 or SEQ ID NO: 10, such as 1, 2, 3, or all four positions (e.g., having K79R, K112R, K148R, and K152R substitutions)), and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic

Viral Vectors, Cold Spring Harbor Laboratory, Gluzman ed., 1982). One of skill in the art can readily use an expression systems such as plasmids and vectors of use in producing mutated SIRT5 proteins in cells including higher eukaryotic cells such as the COS, CHO, HeLa and myeloma cell lines.

### Cells Expressing or Containing a Mutated SIRT5 Protein

A nucleic acid molecule encoding a mutated SIRT5 protein disclosed herein, can be used to transform cells and make transformed cells. In other examples, a mutated SIRT5 protein, such as one including a cell penetrating peptide, is administered to a subject, such that cells of the subject uptake the mutated SIRT5 protein. Thus, cells expressing or containing a mutated SIRT5 protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions), are disclosed. Cells expressing or containing a mutated SIRT5 protein disclosed herein can be eukaryotic or prokaryotic. Examples of such cells include, but are not limited to bacteria, archea, plant, fungal, yeast, insect, and mammalian cells, such as Lactobacillus, Lactococcus, Bacillus (such as B. subtilis), Escherichia (such as E. coli), Clostridium, Saccharomyces or Pichia (such as S. cerevisiae or P. pastoris), Kluyveromyces lactis, Salmonella typhimurium, SF9 cells, C129 cells, 293 cells, Neurospora, and immortalized mammalian myeloid and lymphoid cell lines. In one example the cell is a mammalian liver cell.

Cells expressing a mutated SIRT5 protein can be transformed or recombinant cells. Such cells can include at least one exogenous nucleic acid molecule that encodes a mutated SIRT5 protein, for example a nucleic acid molecule encoding a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions. It is understood that all progeny may not be identical to the parental cell since there may be mutations that occur during replication. A method of stable transfer, meaning that the foreign DNA is continuously maintained in the host cell, can be used.

Transformation of a host cell with recombinant DNA may be carried out by conventional techniques. Where the host is prokaryotic, such as E. coli, competent cells which are capable of DNA uptake can be prepared from cells harvested after exponential growth phase and subsequently treated, for example by the CaC1₂ method. Alternatively, MgC1₂ or RbC1 can be used. Transformation can also be performed after forming a protoplast of the host cell if desired, or by electroporation. Techniques for the propagation of mammalian cells in culture are known (see, Jakoby and Pastan (eds), 1979, Cell Culture. Methods in Enzymology, volume 58, Academic Press, Inc., Harcourt Brace Jovanovich, N.Y.). Exemplary mammalian host cell lines are VERO cells, HeLa cells, CHO cells, and WI38, BHK, and COS cell lines, although cell lines may be used, such as cells designed to provide higher expression desirable glycosylation patterns, or other features. Techniques for the transformation of yeast cells, such as polyethylene glycol transformation, protoplast transformation and gene guns, can also be utilized.

### Pharmaceutical Compositions Containing Mutated SIRT5

Pharmaceutical compositions that include a mutated SIRT5 protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4 or SEQ ID NO: 10, wherein the protein retains an arginine at one or more of positions 79, 112, 148, and 152 of SEQ ID NO: 4, such as at all four positions), or a nucleic acid molecule encoding a mutated SIRT5 protein (such as a mRNA, plasmid, or viral vector, such as a nucleic acid molecule having at least 90%, at least 91%, at least 92%, at least 93%, at least 94%, at least 95%, at least 96%, at least 97%, at least 98%, at least 99%, or 100% sequence identity to any of SEQ ID NOS: 3, 9, 11 and 12) can be formulated with an appropriate pharmaceutically acceptable carrier, depending upon the particular mode of administration chosen.

In some embodiments, the pharmaceutical composition consists essentially of a mutated SIRT5 protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4 or SEQ ID NO: 10, wherein the protein retains an arginine at one or more of positions 79, 112, 148, and 152 of SEQ ID NO: 4 or 10, such as at all four positions), or a nucleic acid molecule encoding a mutated SIRTS protein (such as a mRNA, plasmid, or viral vector, such as a nucleic acid molecule having at least 90%, at least 91%, at least 92%, at least 93%, at least 94%, at least 95%, at least 96%, at least 97%, at least 98%, at least 99%, or 100% sequence identity to any one of SEQ ID NOS: 3, 9, 11 and 12) and a pharmaceutically acceptable carrier. In these embodiments, additional therapeutically effective agents are not included in the compositions.

In other embodiments, the pharmaceutical composition includes a mutated SIRTS protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4 or SEQ ID NO: 10, wherein the protein retains an arginine at one or more of positions 79, 112, 148, and 152 of SEQ ID NO: 4 or SEQ ID NO: 10, such as at all four positions), or a nucleic acid molecule encoding a mutated SIRTS protein (such as a mRNA, plasmid, or viral vector, such as a nucleic acid molecule having at least 90%, at least 91%, at least 92%, at least 93%, at least 94%, at least 95%, at least 96%, at least 97%, at least 98%, at least 99%, or 100% sequence identity to any one of SEQ ID NOS: 3, 9, 11 and 12) and a pharmaceutically acceptable carrier. Additional therapeutic agents, such as agents for the treatment or management of an OA (such as MMA or PA), can be included. Thus, the pharmaceutical compositions can include a therapeutically effective amount of another agent. Examples of such agents include, without limitation, L-carnitine, hydroxycobalamin, vitamin B12, an antibiotic (e.g., metronidazole), sodium benzoate, N-carbamylglutamate or combinations thereof for MMA, or L-carnitine, vitamin B12, an antibiotic (e.g., metronidazole), or combinations thereof, for PA. In some examples, the pharmaceutical compositions containing a mutated SIRTS protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4 or SEQ ID NO: 10, wherein the protein retains an arginine at one or more of positions 79, 112, 148, and 152 of SEQ ID NO: 4 or SEQ ID NO: 10, such as at all four positions), or a nucleic acid molecule encoding a mutated SIRTS protein (such as a mRNA, plasmid, or viral vector, such as a nucleic acid molecule having at least 90%, at least 91%, at least 92%, at least 93%, at least 94%, at least 95%, at least 96%, at least 97%, at least 98%, at least 99%, or 100% sequence identity to any one of SEQ ID NOS: 3, 9, 11 and 12) can further include a therapeutically effective amount of other enzyme (or enzyme coding sequence) missing or defective in the OA patient, such as MMUT, MMAA, MMAB, MMACHC, MMACHD, LMBRD1, or MCEE in an MMA patient, or PCCA (propionyl-CoA carboxylase subunit alpha) or PCCB (propionyl-CoA carboxylase subunit beta) in a PA patient.

The pharmaceutically acceptable carriers and excipients useful in this disclosure are conventional. See, e.g., Remington: The Science and Practice of Pharmacy, The University of the Sciences in

Philadelphia, Editor, Lippincott, Williams, & Wilkins, Philadelphia, PA, 21St Edition (2005). For instance, parenteral formulations usually include injectable fluids that are pharmaceutically and physiologically acceptable fluid vehicles such as water, physiological saline, other balanced salt solutions, aqueous dextrose, glycerol or the like. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example sodium acetate or sorbitan monolaurate. Excipients that can be included are, for instance, other proteins, such as human serum albumin or plasma preparations.

In some embodiments, a mutated SIRTS protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4 or SEQ ID NO: 10, wherein the protein retains an arginine at one or more of positions 79, 112, 148, and 152 of SEQ ID NO: 4 or SEQ ID NO: 10, such as at all four positions), or a nucleic acid molecule encoding a mutated SIRTS protein (such as a mRNA, plasmid, or viral vector, such as a nucleic acid molecule having at least 90%, at least 91%, at least 92%, at least 93%, at least 94%, at least 95%, at least 96%, at least 97%, at least 98%, at least 99%, or 100% sequence identity to any one of SEQ ID NOS: 3, 9, 11 and 12) is included in a controlled release formulation, for example, a microencapsulated formulation. Various types of biodegradable and biocompatible polymers, methods can be used, and methods of encapsulating a variety of synthetic compounds, proteins and nucleic acids, have been well described in the art (see, for example, U.S. Patent Publication Nos. 2007/0148074; 2007/0092575; and 2006/0246139; U.S. Pat. Nos. 4,522, 811; 5,753,234; and 7,081,489; PCT Publication No. WO/2006/052285; Benita, Microencapsulation: Methods and Industrial Applications, 2^{nd} ed., CRC Press, 2006).

In other embodiments, a mutated SIRTS protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4 or SEQ ID NO: 10, wherein the protein retains an arginine at one or more of positions 79, 112, 148, and 152 of SEQ ID NO: 4 or SEQ ID NO: 10, such as at all four positions), or a nucleic acid molecule encoding a mutated SIRTS protein (such as a mRNA, plasmid, or viral vector, such as a nucleic acid molecule having at least 90%, at least 91%, at least 92%, at least 93%, at least 94%, at least 95%, at least 96%, at least 97%, at least 98%, at least 99%, or 100% sequence identity to any one of SEQ ID NOS: 3, 9, 11 and 12) is included in a nanodispersion system. See, e.g., U.S. Pat. No. 6,780,324; U.S. Pat. Publication No. 2009/0175953. For example, a nanodispersion system includes a biologically active agent and a dispersing agent (such as a polymer, copolymer, or low molecular weight surfactant). Exemplary polymers or copolymers include polyvinylpyrrolidone (PVP), poly(D,L-lactic acid) (PLA), poly(D,L-lactic-co-glycolic acid (PLGA), poly(ethylene glycol). Exemplary low molecular weight surfactants include sodium dodecyl sulfate, hexadecyl pyridinium chloride, polysorbates, sorbitans, poly(oxyethylene) alkyl ethers, poly(oxyethylene) alkyl esters, and combinations thereof. In one example, the nanodispersion system includes PVP and ODP or a variant thereof (such as 80/20 w/w). In some examples, the nanodispersion is prepared using the solvent evaporation method, see for example, Kanaze et al., Drug Dev. Indus. Pharm. 36:292-301, 2010; Kanaze et al., J. Appl. Polymer Sci. 102:460-471, 2006. With regard to the administration of nucleic acids, one approach is direct treatment with plasmid DNA, such as with a mammalian expression plasmid. In one example, the composition includes an mRNA encoding the mutant SIRTS protein (such as a nucleic acid molecule having at least 90%, at least 91%, at least 92%, at least 93%, at least 94%, at least 95%, at least 96%, at least 97%, at least 98%, at least 99%, or 100% sequence identity to any one of SEQ ID NOS: 3, 9, 11 and 12) as part of a LNP. As described above, the nucleotide sequence encoding a mutated SIRTS protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4 or SEQ ID NO: 10, wherein the protein retains an arginine at one or more of positions 79, 112, 148, and 152 of SEQ ID NO: 4 or SEQ ID NO: 10, such as at all four positions), such as a nucleic acid molecule having at least 90%, at least 91%, at least 92%, at least 93%, at least 94%, at least 95%, at least 96%, at least 97%, at least 98%, at least 99%, or 100% sequence identity to any one of SEQ ID NOS: 3, 9, 11 and 12, can be placed under the control of a promoter, for example to increase expression of the mutant SIRTS protein.

In one example, a viral vector, such as AAV, is used to deliver a mutant SIRTS coding sequence (such as a nucleic acid molecule having at least 90%, at least 91%, at least 92%, at least 93%, at least 94%, at least 95%, at least 96%, at least 97%, at least 98%, at least 99%, or 100% sequence identity to any one of

SEQ ID NOS: 3, 9, 11 and 12), for example as part of a LNP. The nucleotide sequence encoding a mutated SIRTS protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4 or SEQ ID NO: 10, wherein the protein retains an arginine at one or more of positions 79, 112, 148, and 152 of SEQ ID NO: 4 or SEQ ID NO: 10, such as at all four positions), such as a nucleic acid molecule having at least 90%, at least 91%, at least 92%, at least 93%, at least 94%, at least 95%, at least 96%, at least 97%, at least 98%, at least 99%, or 100% sequence identity to any one of SEQ ID NOS: 3, 9, 11 and 12, can be placed under the control of a promoter, for example to increase expression of the mutant SIRTS protein (for example in the liver). In one example, the dose of AAV encoding the mutant SIRTS protein is at least 1×10¹⁰ genome copies/kg (GC/kg), at least 1×10¹¹ GC/kg, at least 1×10¹² GC/kg, at least 1×10¹³ GC/kg, or at least 1×10¹⁴ GC/kg, such as 1×10¹⁰-1×10¹⁴ GC/kg, 1×10¹⁰-1×10¹³ GC/kg, or 2×10¹⁰-2×10¹³.

Many types of release delivery systems can be used. Examples include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems, such as lipids including sterols such as cholesterol, cholesterol esters and fatty acids or neutral fats such as mono-di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which a mutated SIRT5 protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions), or a nucleic acid molecule encoding such a mutated SIRTS protein (such as a nucleic acid molecule having at least 90%, at least 91%, at least 92%, at least 93%, at least 94%, at least 95%, at least 96%, at least 97%, at least 98%, at least 99%, or 100% sequence identity to any one of SEQ ID NOS: 3, 9, 11 and 12), is contained in a form within a matrix such as those described in U.S. Pat. Nos. 4,452,775; 4,667,014; 4,748,034; 5,239,660; and 6,218,371 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

Use of a long-term sustained release implant may be particularly suitable for treatment of chronic conditions, such as an OA, such as MMA or PA. Long-term release, as used herein, means that the implant is constructed and arranged to deliver therapeutic levels of the active ingredient for at least 30 days, such as at least 60 days, at least 90 days, or at least 120 days. Long-term sustained release implants include some of the release systems described above. These systems have been described for use with nucleic acids (see U.S. Pat. No. 6,218,371). For use in vivo, nucleic acids and peptides can be relatively resistant to degradation (such as via endo- and exo-nucleases). Thus, modifications of the disclosed mutated SIRTS proteins, such as the inclusion of a C-terminal amide, can be used.

The dosage form of the pharmaceutical composition can be determined by the mode of administration chosen. For instance, in addition to injectable fluids, topical, inhalation, oral and suppository formulations can be employed. Topical preparations can include eye drops, ointments, sprays, patches and the like. Inhalation preparations can be liquid (e.g., solutions or suspensions) and include mists, sprays and the like. Oral formulations can be liquid (e.g., syrups, solutions or suspensions), or solid (e.g., powders, pills, tablets, or capsules). Suppository preparations can also be solid, gel, or in a suspension form. For solid compositions, conventional non-toxic solid carriers can include pharmaceutical grades of mannitol, lactose, cellulose, starch, or magnesium stearate. Actual methods of preparing such dosage forms are known, or will be apparent, to those skilled in the art.

The pharmaceutical compositions that include a mutated SIRT5 protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4 or SEQ ID NO: 10, wherein the protein retains an arginine at one or more of positions 79, 112, 148, and 152 of SEQ ID NO: 4 or SEQ ID NO: 10, such as at all four positions) can be formulated in unit dosage form, suitable for individual administration of precise dosages. In one non-limiting example, a unit dosage contains from about 1 mg to about 1 g of a mutated SIRT5 protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions), such as at least 1 mg, at least 10 mg, at least 100 mg, or at least 1 g, such as about 10 mg to about 100 mg, about 50 mg to about 500 mg, about 100 mg to about 900 mg, about 250 mg to about 750 mg, or about 400 mg to about 600 mg. In other examples, a therapeutically effective amount of a mutated SIRT5 protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4 or SEQ ID NO: 10, wherein the protein retains an arginine at one or more of positions 79, 112, 148, and 152 of SEQ ID NO: 4 or SEQ ID NO: 10, such as at all four positions), is about 0.01 mg/kg to about 50 mg/kg, for example, about 0.5 mg/kg to about 25 mg/kg or about 1 mg/kg to about 10 mg/kg. In other examples, a therapeutically effective amount of a mutated SIRT5 protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4 or SEQ ID NO: 10, wherein the protein retains an arginine at one or more of positions 79, 112, 148, and 152 of SEQ ID NO: 4 or SEQ ID NO: 10, such as at all four positions) is about 1 mg/kg to about 5 mg/kg, for example about 2 mg/kg. In a particular example, a therapeutically effective amount of a mutated SIRT5 protein (such as a protein having at least 90%, at least 91%, at least 92%, at least 93%, at least 94%, at least 95%, at least 96%, at least 97%, at least 98%, at least 99%, or 100% sequence identity to

SEQ ID NO: 4 or 10, wherein the protein retains an arginine at one or more of positions 79, 112, 148, and 152 of SEQ ID NO: 4 or 10, such as at all four positions) includes about 1 mg/kg to about 10 mg/kg, such as about 2 mg/kg.

### Treatment of OA Using Mutated SIRT5

The disclosed mutated SIRT5 proteins (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions), or a nucleic acid molecule encoding such a mutated SIRT5 protein (such as a nucleic acid molecule having at least 90%, at least 91%, at least 92%, at least 93%, at least 94%, at least 95%, at least 96%, at least 97%, at least 98%, at least 99%, or 100% sequence identity to any one of SEQ ID NOS: 3, 9, 11 and 12), can be administered to a subject at a therapeutically effective dose, for example to treat an OA, such as MMA, IVA, GA1, or PA, for example by increasing the deacylation (e.g., depropionylation, demethylmalonylation, and/or demalonylion) of proteins, such as enzymes in pathways involved in such disorders, such as CPS1 and GCSH. In one example, the method decreases aberrant methylmalonylation in the treated subject. In one example, the method decreases blood ammonia levels in the treated subject.

### Modes of Administration

The compositions of this disclosure that include a mutated SIRT5 protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions), or a nucleic acid molecule encoding such a mutated SIRT5 protein, can be administered to humans or other animals by any means, including orally, intravenously, intramuscularly, intraperitoneally, intranasally, intradermally, intrathecally, subcutaneously, via inhalation or via suppository. In one non-limiting example, the composition is administered via injection. In some examples, site-specific administration of the composition can be used, for example by administering a mutated SIRT5 protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions), or a nucleic acid molecule encoding such a mutated SIRT5 protein, to liver tissue (for example by using a pump, or by implantation of a slow release form at the site of the liver). The particular mode of administration and the dosage regimen can be selected by the attending clinician, taking into account the particulars of the case (e.g. the subject, the disease, the disease state involved, the particular treatment, and whether the treatment is prophylactic). Treatment can involve daily or multi-daily or less than daily (such as weekly, monthly, bi-monthly, yearly, quarterly, etc.) doses over a period of a few days to months, or even years. For example, a therapeutically effective amount of a mutated SIRT5 protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions), or a nucleic acid molecule encoding such a mutated SIRT5 protein, can be administered in a single dose, for example daily, weekly, monthly, bi-monthly, quarterly, or yearly, or in several doses, for example two or more doses (such as 2, 3, 4, 5 or 6 doses) daily, weekly, monthly, quarterly, or yearly. In a particular non-limiting example, treatment involves a monthly dose. In a particular non-limiting example, treatment involves a dose administered at a time of instability for the patient. The amount of mutated SIRT5 protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions), or a nucleic acid molecule encoding such a mutated SIRT5 protein, administered can be dependent on the subject being treated, the severity of the affliction, and the manner of administration. Within these bounds, the formulation to be administered will contain a quantity of the mutated SIRT5 protein (or nucleic acid sequence encoding such) in amounts effective to achieve the desired effect in the subject being treated. A therapeutically effective amount of mutated SIRT5 protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions), or a nucleic acid molecule encoding such a mutated SIRTS protein, can be the amount is necessary to treat the OA, such as MMA or PA, such as an amount needed to increase the de-acylation (e.g., de-propionylation, de-methylmalonylation, and/or de-malonylation) of proteins, such as enzymes in pathways involved in such disorders, such as CPS1 (for example an increase of at least 5%, at least 10%, at least 20%, at least 30%, at least 40%, at least 50%, at least 60%, at least 75%, at least 90%, at least 95%, at least 100%, at least 200%, at least 300%, at least 400%, or at least 500%, for example relative to no administration of mutant SIRTS). In one example, a therapeutically effective amount of mutated SIRTS protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions), or a nucleic acid molecule encoding such a mutated SIRTS protein, can be the amount necessary to treat the OA, such as MMA or PA, such as an amount needed to decrease aberrant methylmalonylation of proteins, such as enzymes in pathways involved in such disorders, such as CPS1 (for example a decrease of at least 5%, at least 10%, at least 20%, at least 30%, at least 40%, at least 50%, at least 60%, at least 75%, at least 90%, at least 95%, or at least 100%, for example relative to no administration of mutant SIRTS). In one example, a therapeutically effective amount of mutated SIRTS protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions), or a nucleic acid molecule encoding such a mutated SIRTS protein, can be the amount necessary to treat the OA, such as MMA or PA, such as an amount needed to decrease blood ammonia level in the treated subject, for example a decrease of at least 5%, at least 10%, at least 20%, at least 30%, at least 40%, at least 50%, at least 60%, at least 75%, or at least 90%, for example relative to no administration of mutant SIRTS).

### Exemplary Dosages

In one example, a dose of about 1 mg to about 1 g of a mutated SIRTS protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions), such as at least 1 mg, at least 10 mg, at least 100 mg, or at least 1 g, such as about 10 mg to about 100 mg, about 50 mg to about 500 mg, about 100 mg to about 900 mg, about 250 mg to about 750 mg, or about 400 mg to about 600 mg is administered to the subject. In other examples, a therapeutically effective amount of a mutated SIRTS protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions), is about 0.01 mg/kg to about 50 mg/kg, for example, about 0.5 mg/kg to about 25 mg/kg or about 1 mg/kg to about 10 mg/kg. In other examples, a therapeutically effective amount of a mutated SIRTS protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions) is about 1 mg/kg to about 5 mg/kg, for example about 2 mg/kg. In a particular example, a therapeutically effective amount of a mutated SIRTS protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions) includes about 1 mg/kg to about 10 mg/kg, such as about 2 mg/kg.

When a viral vector is utilized for administration of an nucleic acid encoding a mutated SIRTS protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions), such as a vector having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 11 or 12 (and encode a mutant SIRT protein having an arginine at one or more of positions 79, 112, 148, and 152 of SEQ ID NO: 4, such as at all four positions) the recipient can receive a dosage of each recombinant virus in the composition in the range of from about 1×10¹⁰ to about 1×10¹⁴ GC/kg, although a lower or higher dose can be administered. Examples of methods for administering the composition into mammals include, but are not limited to, exposure of cells to the recombinant virus ex vivo, or injection of the composition into the affected tissue or intravenous, subcutaneous, intradermal or intramuscular administration of the virus. In one example, a recombinant viral vector or combination of recombinant viral vectors can be administered locally by direct injection into the liver, for example in a pharmaceutically acceptable carrier. In some examples, the viral vector encoding the mutant SIRTS protein is targeted to a particular tissue, such as the liver (e.g., SEQ ID NOS: 11 and 12). In some examples, the viral vector encoding the mutant SIRTS protein is not targeted to a particular tissue (e.g., is generally expressed throughout the body).

Generally, the quantity of recombinant viral vector, carrying the mutated SIRTS coding sequence administered (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions) is based on the titer of virus particles. An exemplary dose administered to a subject having an OA (such as isolated MMA or PA) is at least 10⁵, at least 10⁶, at least 10⁷, at least 10⁸, at least 10⁹,or at least 10¹⁰ virus particles per mammal, such as a human. Another exemplary dose administered to a subject having an OA (such as isolated MMA or PA) is at least 1×10¹⁰ genome copies per kg (gc/kg), such as at least 1×10¹¹ gc/kg, at least 1×10¹² gakg, at least 1×10¹³gc/kg, or at least 1×10¹⁴ gc/kg, such as 1×10¹² gc/kg to 1×10¹³gc/kg or 1×10¹⁰ gc/kg to 1×10²⁰ gc/kg.

In one example, an mRNA encoding a mutated SIRTS protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions) is administered to the subject having an OA (such as isolated MMA or PA), for example as part of a lipid nanoparticle (LNP), such as lipofectamine (Lf), LNP lacking cationic lipids (nLNPs), a cationic LNP (cLNP) or ionizable cationic LNP (icLNP) (see for example Truong et al., PNAS 116:21150-9, 2019, herein incorporated by reference in its entirety). In some example, the LNP has an average diameter of at least 100 nm, at least 150 nm, at least 200 nm, or at least 300 nm, such as 100 nm to 200 nm. In one example, a dose of at least 0.05 mg/kg, at least 0.5 mg/kg, at least 1 mg/kg, or at least 2 mg/kg is used, such as 0.05 mg/kg to 2 mg/kg could be used

### Administration of Additional Agents/Treatments

In some examples, a mutated SIRTS protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions), or a nucleic acid encoding the mutated SIRTS protein, is administered in combination (such as sequentially or simultaneously or contemporaneously) with one or more other agents, such as those useful in the treatment of OA (such as isolated MMA or PA) or one or more symptoms thereof.

In some examples, a mutated SIRTS protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions), or a nucleic acid encoding the mutated SIRTS protein, can be administered in combination with effective doses of other therapeutic agents (such as those currently used to treat MMA or PA). The term “administration in combination” or “co-administration” refers to both concurrent and sequential administration of the active agents. Administration of mutated SIRT5 protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions) or a nucleic acid encoding such a mutant SIRT5 protein, may also be in combination with a low fat, high calorie diet and/or a diet that avoids isoleucine, valine, threonine, and methionine.

Additional agents that can be used in combination with the disclosed mutated SIRT5 proteins to treat MMA include, without limitation, the missing enzyme in the subject (for example as a protein, nucleic acid encoding the enzyme, such as a vector and/or mRNA encoding the enzyme, such as MMUT, MMAA, MMAB, MMACHC, MMACHD, LMBRD1, or MCEE); L-carnitine (e.g., enterally administered at 50-100mg/kg/day); hydroxycobalamin (e.g., 1 mg, intramuscularly daily); vitamin B12; one or more antibiotics (for example to reduce intestinal flora, e.g., oral metronidazole); one or more probiotics; sodium benzoate; N-carbamylglutamate; or combinations thereof. In some examples, the MMA subject has previously received a liver and/or kidney transplant. In some examples, the MMA subject could have previously received conventional AAV gene addition therapy, AAV mediated MMUT gene editing at albumin, systemic lentiviral gene therapy targeting the liver, or hybrid transposon AAV gene therapy

Additional agents that can be used in combination with the disclosed mutated SIRTS proteins to treat PA include, without limitation, the missing enzyme in the subject (for example as a protein, nucleic acid encoding the enzyme, such as a vector encoding the enzyme, such as PCCA (propionyl-CoA carboxylase subunit alpha), or PCCB (propionyl-CoA carboxylase subunit beta)); L-carnitine (e.g., enterally administered at 50-100mg/kg/day); vitamin B12; one or more antibiotics (e.g., oral metronidazole); or combinations thereof. In some examples, the MMA subject has previously received a liver and/or kidney transplant.

### Exemplary Subjects

In some embodiments, methods are provided for treating MMA, for example by reducing methylmalonic acid levels in the blood and/or urine, in a subject by administering a therapeutically effective amount of a composition including a mutated SIRTS protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions), or a nucleic acid encoding the mutated SIRTS protein, to the subject. In some embodiments, the method for treating MMA, increases the de-acylation (e.g., de-propionylation, de-methylmalonylation and/or de-malonylation) of proteins, such as enzymes in pathways involved in such disorders, such as CPS1 and/or GCSH (for example increases de-acylation of such proteins by at least 5%, at least 10%, at least 20%, at least 30%, at least 40%, at least 50%, at least 60%, at least 75%, at least 90%, at least 95%, at least 100%, at least 200%, at least 300%, at least 400%, or at least 500%, for example relative to no administration of mutant SIRTS). The subject can have any MMA, such as isolated MMA. The subject can be any mammalian subject, including human subjects and veterinary subjects such as cats and dogs. The subject can be a child or an adult. The subject can also be administered one or more other treatments for OA (such as one or more of those provided herein). The method can include measuring blood or urine methylmalonic levels.

In some examples, the method includes selecting a subject with MMA, such as isolated MMA. These subjects can be selected for treatment with a disclosed mutant SIRT5 protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions), or a nucleic acid encoding the mutated SIRT5 protein. In some examples, a subject with MMA may be selected for treatment or diagnosis with the disclosed methods, by detecting elevated methylmalonic acid in the blood or urine, as shown Table 1 (see Keyfi et al., Rep Biochem Mol Biol. 2016 Oct; 5(1): 1-14, herein incorporated by reference in its entirety). In some examples, the method includes measuring or detecting propionylation, malonylation or methylmalonylation on one or more enzymes, such as CPS1 or GCSH in a sample from the subject, such as a blood, urine or liver sample.

TABLE 1
Methylmalonic acid concentrations in
urine for different subtypes of MMA

Methylmalonic acid concentration

| | Urine | |
|---|---|---|
| Subtype | (mmol/mol creatinine) | Blood (μM) |
| mut-, mut 0 | 1000-10000 | 100-1000 |
| cblB, cblA, cblD | 10-100 | 5-100 |
| MCEE deficiency, | 50-1500 | 7 |
| SUCLA2 | | |
| Normal | <4 | <0.27 |

Thus, in some examples, a subject with MMA treated or diagnosed using the methods provided herein has a concentration of methylmalonic acid in the urine of at least 10 mmol/mol creatinine, at least 50 mmol/mol creatinine, at least 100 mmol/mol creatinine, at least 1000 mmol/mol creatinine, or at least 5000 mmol/mol creatinine, prior to treatment. In some examples, a subject with MMA treated or diagnosed using the methods provided herein have a concentration of methylmalonic acid in the blood of at least 5 μM, at least 7 μM, at least 10 μM, at least 50 μM, or at least 100 μM, prior to treatment. Thus, in some examples, the disclosed methods of treatment or diagnosis can include measuring the concentration of methylmalonic acid in the urine or blood. In some examples, a subject with MMA treated or diagnosed using the methods provided herein have elevated levels of ketone bodies, such as acetone, in the blood (ketonemia) or in the urine (ketonuria) prior to treatment. In some examples, a subject with MMA treated or diagnosed using the methods provided herein have an elevated level of ammonia in the blood (hyperammonemia) prior to treatment. In some examples, a subject with MMA treated or diagnosed using the methods provided herein have excessive levels of glycine in the blood (hyperglycinemia) and in the urine (hyperglycinuria) prior to treatment. In some examples, a subject with MMA treated or diagnosed using the methods provided herein have a lower concentration than normal of white blood cells, blood platelets and red blood cells prior to treatment. In some examples, a subject with MMA treated or diagnosed using the methods provided herein have low blood sugar (hypoglycemia) prior to treatment.

In some embodiments, methods are provided for treating PA, for example by reducing propionyl-CoA, C3 (propionylcarnitine), propionic acid, ketones, ammonia, and/or plasma amino acids (e.g., glycine) in the blood and/or 3-hydroxypropionate, methylcitrate, tiglylglycine, propionylglycine, and/or lactic acid urine, in a subject by administering a therapeutically effective amount of a composition including a mutated SIRT5 protein (such as a protein having at least 90%, at least 91%, at least 92%, at least 93%, at least 94%, at least 95%, at least 96%, at least 97%, at least 98%, at least 99%, or 100% sequence identity to SEQ ID

NO: 4, wherein the protein retains an arginine at one or more of positions 79, 112, 148, and 152 of SEQ ID NO: 4, such as at all four positions), or a nucleic acid encoding the mutated SIRT5 protein, to the subject. In some embodiments, the method for treating PA increases the de-acylating, de-propionylating, de-methylmalonyllysating, and/or de-malonyllysating of proteins, such as enzymes in pathways involved in such disorders, such as CPS1 and/or GCSH (for example an increase of at least 5%, at least 10%, at least 20%, at least 30%, at least 40%, at least 50%, at least 60%, at least 75%, at least 90%, at least 95%, at least 100%, at least 200%, at least 300%, at least 400%, or at least 500%, for example relative to no administration of mutant SIRTS). The subject can have PA due to one more mutations in PCCA or PCCB. The subject can be any mammalian subject, including human subjects and veterinary subjects such as cats and dogs. The subject can be a child or an adult. The subject can also be administered insulin. The method can include measuring levels of one or more of propionyl-CoA, C3 (propionylcarnitine), propionic acid, ketones, ammonia, and plasma amino acids (e.g., glycine) in the blood. The method can include measuring levels of one or more of organic acids in urine, such as 3-hydroxypropionate, methylcitrate, tiglylglycine, propionylglycine, and lactic acid.

In some examples, the method includes selecting a subject with PA. These subjects can be selected for treatment with a disclosed mutant SIRTS protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions), or a nucleic acid encoding the mutated SIRTS protein. In some examples, a subject with PA may be selected for treatment or diagnosis with the disclosed methods, by detecting elevated propionyl-CoA, C3 (propionylcarnitine), propionic acid, ketones, ammonia, and/or plasma amino acids (e.g., glycine) in the blood and/or 3-hydroxypropionate, methylcitrate, tiglylglycine, propionylglycine, and/or lactic acid urine. In some examples, the method includes measuring or detecting propionylation, malonylation, or methylmalonylation on one or more enzymes, such as CPS1, SIRTS, and/or GCSH in a sample from the subject, such as a blood, urine or liver sample.

Thus in some examples, a subject with PA treated or diagnosed using the methods provided herein have elevated propionyl-CoA in the blood, elevated C3 (propionylcarnitine) in the blood, elevated propionic acid in the blood, elevated ketones in the blood, elevated ammonia in the blood, elevated plasma amino acids (e.g., glycine), elevated 3-hydroxypropionate in the urine, elevated methylcitrate in the urine, elevated tiglylglycine in the urine, elevated propionylglycine in the urine, and/or elevated lactic acid in the urine, prior to treatment.

### Therapeutic Effects

In some examples, treating MMA includes one or more of increasing body weight of the subject (for example an increase of at least 10%, at least 20%, at least 50%, at least 75%, at least 100%, at least 2-fold, at least 3-fold, at least 4-fold, at least 5-fold, or more, for example relative to no administration of the mutant SIRTS), decreasing aberrant methylmalonylation of proteins in the liver (for example a decrease of at least 10%, at least 20%, at least 50%, or at least 75% or more), decreasing methylmalonic acid in the blood (for example a decrease of at least 10%, at least 20%, at least 50%, or at least 75% or more, for example relative to no administration of the mutant SIRTS), decreasing methylmalonic acid in the urine (for example a decrease of at least 10%, at least 20%, at least 50%, or at least 75%or more, for example relative to no administration of the mutant SIRTS), decreasing ketone bodies (such as acetone) in the blood (for example a decrease of at least 10%, at least 20%, at least 50%, or at least 75%,or more, for example relative to no administration of the mutant SIRTS), decreasing ketone bodies (such as acetone) in the urine (for example a decrease of at least 10%, at least 20%, at least 50%, or at least 75%or more, for example relative to no administration of the mutant SIRTS), decreasing ammonia in the blood (for example a decrease of at least 10%, at least 20%, at least 50%, or at least 75%or more, for example relative to no administration of the mutant SIRTS), decreasing glycine in the blood (for example a decrease of at least 10%, at least 20%, at least 50%, or at least 75% or more, for example relative to no administration of the mutant SIRTS), decreasing glycine in the urine (for example a decrease of at least 10%, at least 20%, at least 50%, or at least 75%or more, for example relative to no administration of the mutant SIRTS), increasing white blood cells in the blood (for example an increase of at least 10%, at least 20%, at least 50%, at least 75%, at least 100%, at least 2-fold, at least 3-fold, at least 4-fold, at least 5-fold, at least 10-fold, at least 50-fold, at least 100-fold, at least 1000-fold, or more, for example relative to no administration of the mutant SIRTS), increasing red blood cells in the blood (for example an increase of at least 10%, at least 20%, at least 50%, at least 75%, at least 100%, at least 2-fold, at least 3-fold, at least 4-fold, at least 5-fold, at least 10-fold, at least 50-fold, at least 100-fold, at least 1000-fold or more, for example relative to no administration of the mutant SIRTS), increasing platelets in the blood (for example an increase of at least 10%, at least 20%, at least 50%, at least 75%, at least 100%, at least 2-fold, at least 3-fold, at least 4-fold, at least 5-fold, at least 10-fold, at least 50-fold, at least 100-fold, at least 1000-fold, or more, for example relative to no administration of the mutant SIRTS), and increasing blood glucose (for example an increase of at least 10%, at least 20%, at least 50%, at least 75%, at least 100%, at least 2-fold, at least 3-fold, at least 4-fold, at least 5-fold, at least 10-fold, at least 50-fold, at least 100-fold, at least 1000-fold or more, for example relative to no administration of the mutant SIRTS).

In some examples, treating PA includes one or more of decreasing propionyl-CoA in the blood (for example a decrease of at least 10%, at least 20%, at least 50%, or at least 75%or more, for example relative to no administration of the mutant SIRTS), decreasing C3 in the blood (for example a decrease of at least 10%, at least 20%, at least 50%, or at least 75%or more, for example relative to no administration of the mutant SIRTS), decreasing propionic acid in the blood (for example a decrease of at least 10%, at least 20%, at least 50%, or at least 75%or more, for example relative to no administration of the mutant SIRTS), decreasing ketones in the blood (for example a decrease of at least 10%, at least 20%, at least 50%, or at least 75% or more, for example relative to no administration of the mutant SIRTS), decreasing ammonia in the blood (for example a decrease of at least 10%, at least 20%, at least 50%, or at least 75% or more, for example relative to no administration of the mutant SIRTS), decreasing plasma amino acids (e.g., glycine) in the blood (for example a decrease of at least 10%, at least 20%, at least 50%, or at least 75% or more, for example relative to no administration of the mutant SIRTS), decreasing 3-hydroxypropionate in the urine (for example a decrease of at least 10%, at least 20%, at least 50%, or at least 75%or more, for example relative to no administration of the mutant SIRTS), decreasing methylcitrate in the urine (for example a decrease of at least 10%, at least 20%, at least 50%, or at least 75%or more, for example relative to no administration of the mutant SIRTS), decreasing tiglylglycine in the urine (for example a decrease of at least 10%, at least 20%, at least 50%, or at least 75% or more, for example relative to no administration of the mutant SIRTS), decreasing propionylglycine in the urine (for example a decrease of at least 10%, at least 20%, at least 50%, or at least 75%or more, for example relative to no administration of the mutant SIRTS), and decreasing lactic acid in the urine (for example a decrease of at least 10%, at least 20%, at least 50%, or at least 75%, or more, for example relative to no administration of the mutant SIRTS).

In some examples, administration of a mutated SIRTS protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions), or nucleic acid molecule encoding such, treats an OA, such as MMA or PA, by increasing the de-acylation (e.g., de-propionylation, de-methylmalonylation and/or de-malonylation) of proteins, such as enzymes in pathways involved in such disorders, such as CPS1 and/or GCSH (for example an increase of at least 5%, at least 10%, at least 20%, at least 30%, at least 40%, at least 50%, at least 60%, at least 75%, at least 90%, at least 95%, at least 100%, at least 200%, at least 300%, at least 400%, or at least 500%, for example relative to no administration of mutant SIRTS).

In some embodiments, the disclosed methods include comparing one or more indicators of an OA (such as hyperpropionylation, hypermethylmalonylation, and/or hypermalonylation of CPS1 and/or GCSH, or methylmalonic acid in the blood or urine, or other indicators provided herein) to a control (such as no administration of a mutated SIRTS protein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions), or a nucleic acid molecule encoding such), wherein an increase or decrease in the particular indicator relative to the control (as discussed above) indicates effective treatment of OA. The control can be any suitable control against which to compare the indicator of OA in a subject. In some embodiments, the control is a sample obtained from a healthy subject (such as a subject without an OA). In some embodiments, the control is a historical control or standard reference value or range of values (such as a previously tested control sample, such as a group of subjects with OA, or group of samples from subjects that do not have an OA). In further examples, the control is a reference value, such as a standard value obtained from a population of normal individuals that is used by those of skill in the art. Similar to a control population, the value of the sample from the subject can be compared to the mean reference value or to a range of reference values (such as the high and low values in the reference group or the 95% confidence interval). In other examples, the control is the subject (or group of subjects) treated with placebo compared to the same subject (or group of subjects) treated with the therapeutic compound in a cross-over study. In further examples, the control is the subject (or group of subjects) prior to treatment.

The disclosure is illustrated by the following non-limiting Examples.

### Example 1

### Identification of Post-Translational Modifications (PTMs) in Isolated MMA

This example describes methods used to identify a novel inhibitory PTM, methylmalonylation, produced as a consequence of MMA and present in abundance in the liver tissue in isolated MMA patients (FIG. 1).

Methylmalonylated BSA was prepared as follows. Briefly, fatty acid free, nonacetylated, ultra-pure

BSA was resuspended in Tris-Cl buffer (50 mM Tris-Cl and 150 mM NaC1 at a pH of 8 at 37 C) at a stock concentration of 10 mg/ml. Methylmalonyl-CoA (Sigma, M1762) was suspended in Tris-Cl solution (same as above) at a stock concentration of 5mM. BSA was then incubated at a concentration of 1 mg/ml with or without increasing concentrations of methylmalonyl-CoA (0.5, 1.0, 1.5, 2.0, and 2.5 mM) in a 40 μ1 reaction volume. Reactions were placed in a thermomixer (Benchmark Multi-Therm™ Heat-Shake) for 9 hours at 37

C for 9 hours at 600 RPM. Reactions were then denatured in SDS, boiled, and run on SDS-PAGE gel. Resulting western blots were stained for succinyl-lysine (PTM biolabs, PTM-401), and then stripped and reprobed with malonyl-lysine (PTM biolabs, PTM-901). Only malonyl-lysine antibody exhibited bi-reactivity to methylmalonyl-lysine on BSA substrate demonstrating methylmalonylation can be placed non-enzymatically on protein substrate under alkaline conditions.

The ability of the methylmalonylated BSA to bind to commercial anti-malonyllysine antibody or anti-succinyllysine antibody (negative control) was examined in vitro. As shown in FIG. 2A, methylmalonylated BSA was recognized by an anti-malonyllysine antibody, but not by anti-succinyllysine antibodies. This demonstrated that the anti-malonyllysine antibody exhibited a bi-reactivity towards methylmalonylation and malonylation, likely due to the high similarity in structure of the two PTMs. Tandem mass spectrometry analysis also indicated this bi-reactivity occurs as there was a mass shift for methylmalonylated peptides consistent with methylmalonylation of lysine compared to malonylated residues (FIG. 3).

Hepatic extracts from Mmut^{-/-}; Tg^{INS-MCK-Mmut} a mouse model of severe MMA, where expression of the methymalonyl-CoA mutase enzyme is limited to the skeletal muscle were next examined with malonyl-lysine antibodies. Whole liver tissue extracts were made by mechanical homogenization of liver tissue from two Mmut^{-/-}; Tg^{INS-MCK-Mmmuat} (Mmut control) and two Mmut^{-/-}; Tg^{INS-MCK-Mmut} (Mmut mutant) mice as well as two control human hepatic extracts (C5/C28) and 2 MMA patient hepatic extracts (P4/P5) in chemical lysis buffer T-PER™ (Thermo Scientific, 78510) with protease inhibitor cocktail (Roche, cOmplete tablets, Mini EDTA-free, easy pack) added. Whole liver tissue extracts were measured and normalized for protein content, boiled in SDS and run on SDS-PAGE gel. The resulting blots were stained with malonyl-lysine antibody and β-actin antibody (Proteintech, 66009-1-1g) to serve as a loading control.

As shown in FIG. 2B, widespread hyper-acylation in the livers of MMA mice, but not littermate controls, was observed. Similar observations were made using liver extracts prepared from isolated MMA patients compared to unrelated liver donor controls (FIG. 2B). That is, liver extracts from isolated MMA patients showed PTM accumulation, while liver extracts from normal patients (controls) did not. Additionally, when the methylmalonyl and malonyl-CoA pools are depleted through loss of Acsf3 expression, there is also reduced methylmalonylation and malonylation. ACSF3 is the enzyme responsible for converting methylmalonic and malonic acid to methylmalonyl-CoA and malonyl-CoA respectively. Without ACSF3, these metabolites accumulate in body fluids without the accumulation of the CoAs as seen in isolated MMUT type MMA. CMAMMA is generally milder than MMA, with neurological manifestations that may not present until adulthood. Acsf3^{-/-}mice do not exhibit isolated MMA pathophysiology and have reduced methylmalonylation and malonylation compared to WT mice and Mmut^{-/-}; Tg^{INS-MCK-Mmut} (FIG. 2C). This genetic model further supports the bireactivity of the antimalonyl-lysine antibody, and shows that the reactivity comes from the respective CoA moieties.

Anti-malonyl antibody columns were used to purify hepatic extracts from Mmut^{-/-}; Tg^{INS-MCK-Mut}, Mmut^{-/-}; Tg^{INS-MCK-Mmut} and WT mice (FIG. 3). Due to its bi-reactivity to methylmalonyl-lysine but not to succinyl-lysine, proteins with malonylation and methylmalonylation PTMs were purified and the type of acylation at each lysine residue on any given protein was distinguished by tandem mass spectrometry analysis. Mass spectrometry was then used to characterize the PTM proteome. As shown in FIGS. 4 and 5A-5B, these IPs followed by tandem mass spectrometry analysis revealed enrichment of malonylation and methylmalonylation PTMs on glutathione S-transferases, urea cycle enzymes, arginine biosynthesis enzymes, and oxidoreductase pathway proteins compared to WT mice.

Based on a murine proteomic analysis performed using the anti-(methyl)malonyllysine antibody to enrich modified proteins in Mmut^{-/-}; Tg^{INS-MCK-Mmut} liver extracts, Cps1/CPS1, a critical enzyme involved in ureagenesis (a pathway that is perturbed in MMA and PA patients), was examined in liver extracts from patients with isolated MMA. Hepatic extracts prepared as described above from three human control and three human MMA patient livers were run on western blot and stained for CPS1 antibody and β-actin antibody (a loading control). Lysates were also immunoprecipitated for CPS1 by conjugating CPS1 antibody (abcam, ab129076) to protein A beads (Roche, 11134515001). Here lysates containing 3 mg of total protein were pre-cleared by 3 hours incubation with protein A bead incubation end-over-end at 4° C. followed by incubation with CPS1 antibody conjugated beads end-over-end overnight at 4° C. The following morning, beads were washed 4 times in lysis buffer, boiled and run on SDS page. The resulting western was stained with malonyl-lysine antibody then stripped and reprobed for total CPS1 levels.

As shown in FIG. 2D, while liver extracts from patients with isolated MMA have increased amounts of CPS1 compared to controls (see top panel of FIG. 2D), the enzyme is heavily modified by methylmalonylation/malonylation (see bottom panel of FIG. 2D). A specific methylmalonylation of lysine 1291 was detected in the MMA mice but not controls (FIG. 5C and FIG. 6). Furthermore, because CPS1 is inactivated by acylation PTMs, these results indicate that in isolated MMA, CPS1 function, and subsequently ureagenesis, may be dysregulated by the PTM:SIRT axis. Hyperammonemia is a known complication experienced by patients with isolated MMA, including MMUT deficiency, and these results suggest urea cycle dysfunction experienced by MMA patients may depend, in part, on aberrant modification of CPS1 by methylmalonylation.

Therefore, disease specific hyper-malonylation/methylmalonylation of CPS1 and other urea cycle enzymes could account for pathway inactivation and hyperammonemia. Inborn errors of metabolism including MMA often also display defects in the Krebs cycle which was originally attributed to inhibition by toxic metabolites or alternatively to reduced expression of Krebs cycle enzymes, however, increased total levels of Krebs cycle enzymes were found in MMA mice compared to controls, likely due to disease related increases in mitochondrial mass and evidence indicated inhibition of this pathway is due to increased acylation PTMs (FIG. 5E). Mass spectrometry analysis revealed increased methylmalonylation on Krebs cycle proteins Mdh2, Idh2, Dlst, and Aco2 and all of these proteins along with Ogdh had increased total levels in hepatic extracts from Mmut^{-/-}; Tg^{INS-MCK-Mmut} mice compared to control mice. Follow up immunoprecipitation analysis confirmed increased malonylation/methylmalonylation on one such protein Idh2 in MMA mice and not controls (FIGS. 5E-5F).

One protein that also stood out on mass spectrometry analysis was Gludl which exhibited both methylmalonylation and malonylation, specifically at lysine 503. Acetylation of this lysine is thought to increase enzymatic activity due to its location in the GTP binding pocket of the enzyme. This pocket is an allosteric binding site, and when occupied by GTP, inhibits enzymatic activity of GLUD1. Acetylation of

K503 may allow for binding to the negatively charged phosphate group of inhibitor GTP, however, acetylation can neutralize the positive charge and lower binding of GTP thus increasing GLUD1 activity. Malonylation or methylmalonylation would not only neutralize the positive charge of K503 but provide a net negative charge which would further repel the negatively charged GTP. Thus, in this instance, increased malonyl or methylmalonylation of K503 would increase enzymatic activity and could contribute to hypoglycemia commonly observed in patients with MMA. The disclosed study confirmed increased malonyl and or methylmalonylation of GLUD1 when purified from MMA patient liver tissue samples compared to control samples (FIG. 5G).

Overall, the mass spectrometry data revealed that aberrant acylation could account for the dysregulation of many metabolic pathways in MMA and therefore contribute to disease pathophysiology.

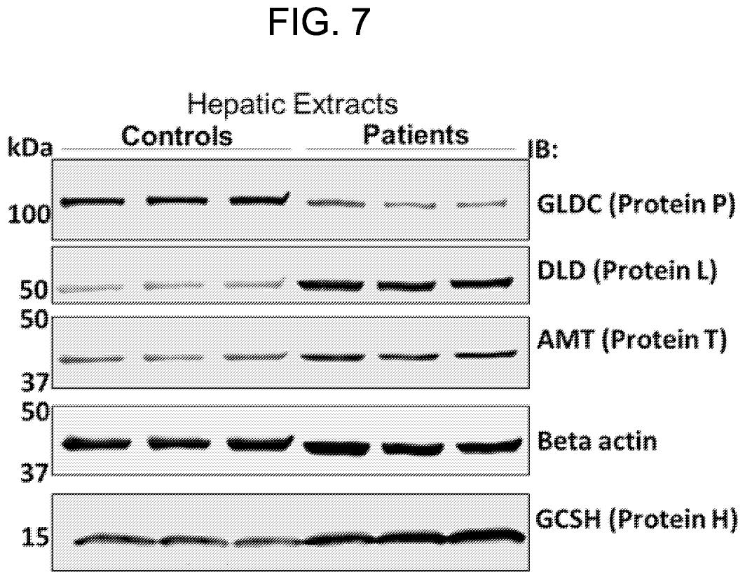

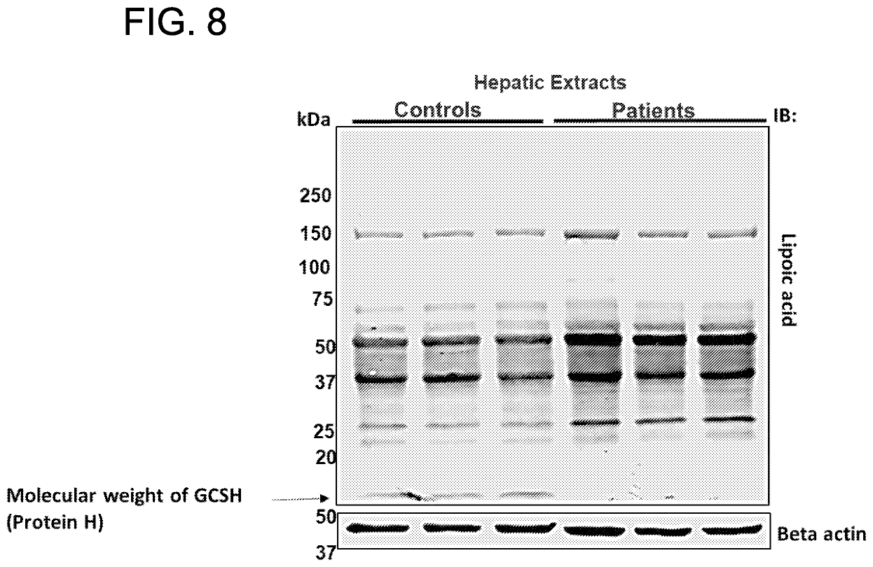

Additionally, liver extracts from patients with isolated MMA have increased methylmalonylation/malonylation of glycine cleavage enzyme, GCSH, which inhibited placement of the activating lipoylation PTM which is seen in control but not patient extracts (FIGS. 7-10). Because hyperacylation of GCSH would prevent placement of lipoic acid, glycine cleavage is dysregulated by this PTM:SIRT axis as well leading to hyperglycinemia.

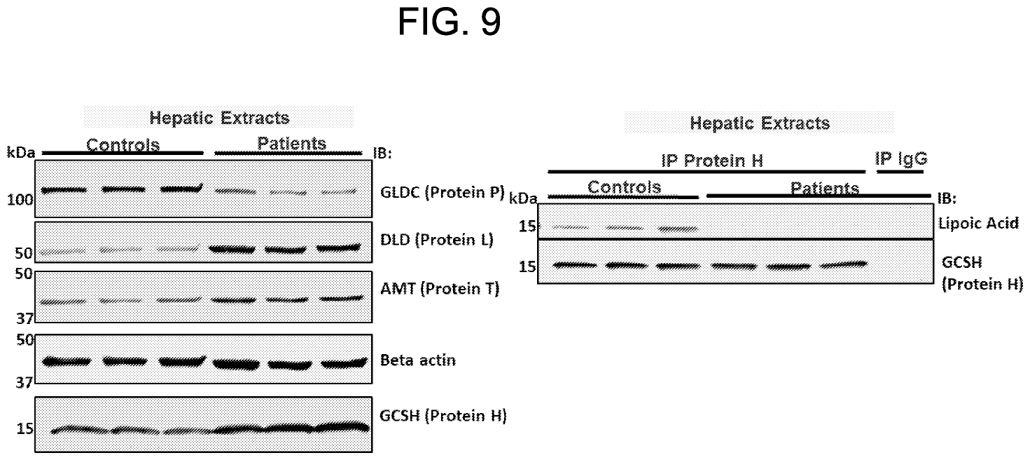

Hepatic extracts prepared as described above from two to three human control and two to three human MMA patient livers were run on western blot and stained for GCSH antibody, DLD antibody, AMT antibody, GCLD antibody, and β-actin antibody (a loading control) (FIG. 7). Next, global lipoylation was assessed by probing the extracts with an anti-lipoic antibody; a specific band suspected to be protein H at—15 kD was missing from the patients, suggesting that while protein H was present in the patient samples (FIG. 7, bottom lane), it was not modified properly. Lysates were also immunoprecipitated for GCSH by conjugating GCSH antibody (ProteinTech 16726-1-AP) to protein A beads (Roche, 11134515001) (FIG. 9).

Here lysates containing 3 to 6 mg of total protein were pre-cleared by 3 hours incubation with protein A bead incubation end-over-end at 4° C. followed by incubation with GCSH antibody conjugated beads end-over-end overnight at 4° C. The following morning, beads were washed 4 times in lysis buffer, boiled and run on SDS page. The resulting western was stained with lipoic acid antibody (abcam, ab58724) then stripped and reprobed for total GCSH levels or with malonyl-lysine antibody then stripped and reprobed for total GCSH levels.

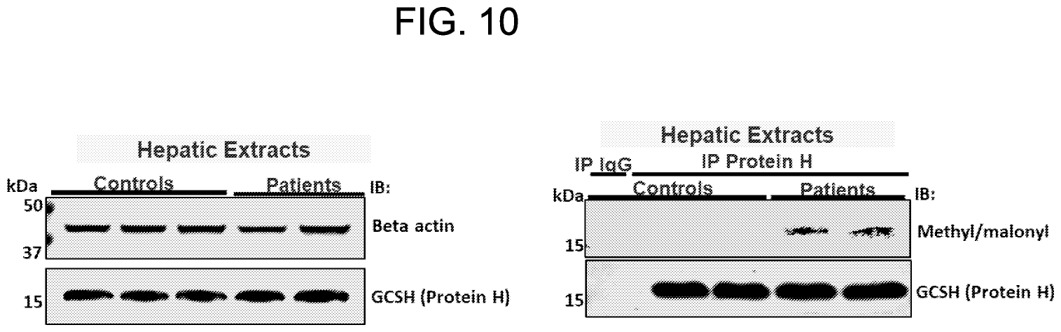

The results show that the patient samples have increased levels of immunoreactive GCSH (FIG. 9, left) that is not properly modified by lipoylation (FIG. 9, right). These same samples were studied for the presence of a PTM using the bi-reactive (methyl)malonyl-lysine antibody (PTM Biolabs, PTM-901) (FIG. 10) and confirm that while GCSH (Protein H) is present and, in contrast to the controls, is modified with an aberrant PTM on the active site lysine residue, the normal site of lipoylation. A model of the aberrant modification is presented in FIG. 11.

Based on these observations, it was concluded that methymalonyllysine is a newly identified PTM in isolated MMA patients, which inactivates CPS1 and GCSH (Protein H), and leads to hyperammonemia and hyperglycinemia.

As mass spectrometry has reduced sensitivity for the detection of low abundance proteins such as proteins involved in mitochondrial DNA stability, transcription, and copy number regulation as well as mitochondrial structure, these pathways and whether they may be targets of aberrant methylmalonylation were further explored.

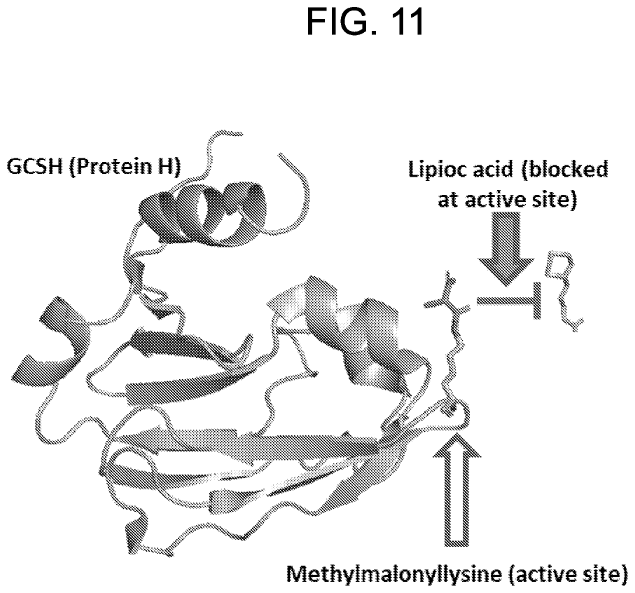

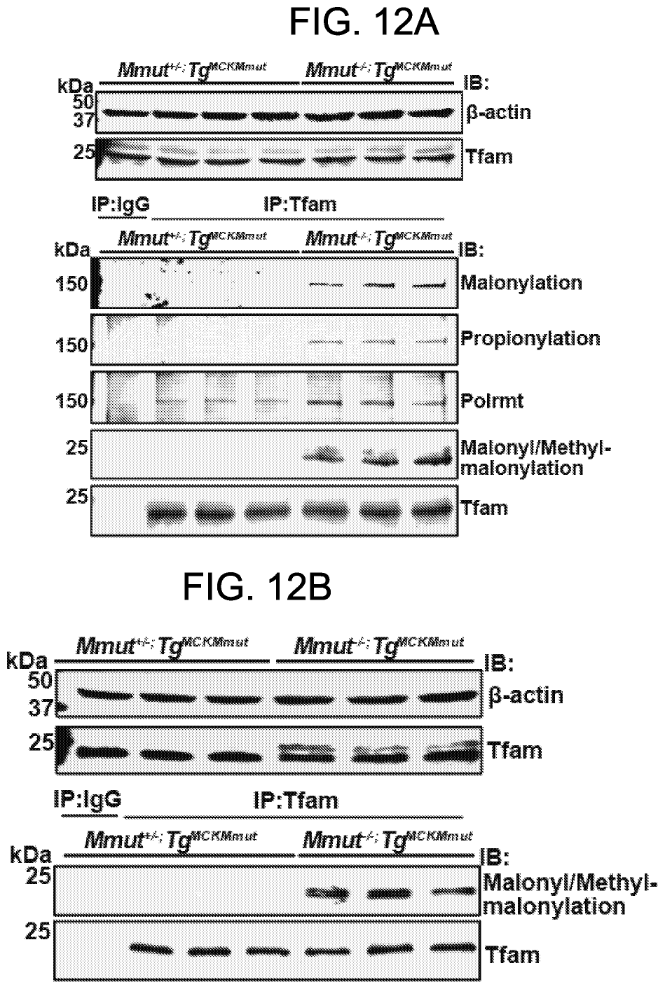

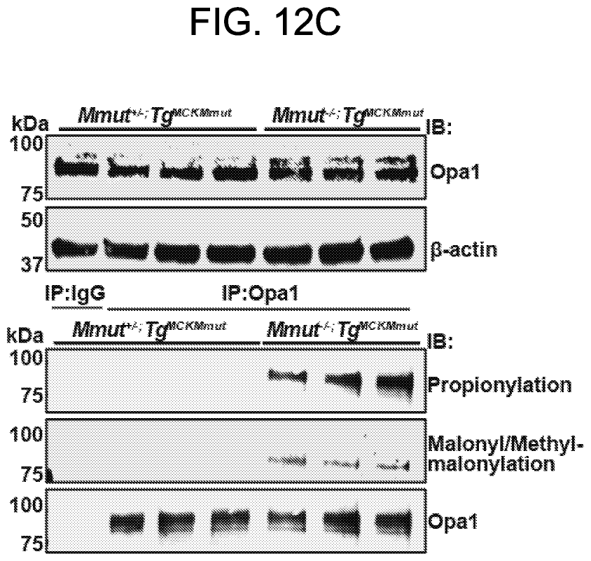

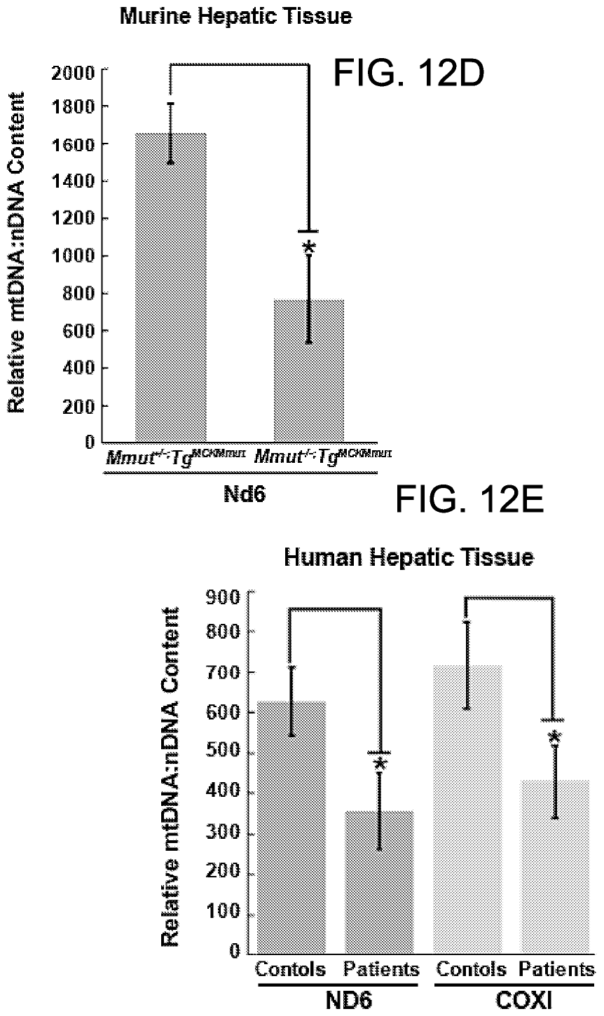

In MMA, megamitochondria have been documented in hepatic and renal tissues and often display loss of cristae structures as well as reduced expression of electron transport chain proteins encoded by mitochondrial DNA (mtDNA). But the underlying mechanisms for these deficiencies were still unclear. It was hypothesized that aberrant acylation of enzymes responsible for mtDNA stability, transcription and copy number as well as enzymes involved in the maintenance of mitochondrial structure would lead to reduced protein functionality and account for these disease phenotypes. First examined was mitochondrial transcription factor A (Tfam), which localizes to the mitochondria where it stabilizes mtDNA and therefore maintains mtDNA copy number and regulates the expression of mtDNA genes such as electron transport chain subunits. Overall Tfam protein levels were not reduced in MMA mouse hepatic or renal tissues compared to controls (FIGS. 12A-12B) indicating loss of mtDNA expression is not the result of reduced Tfam. To determine if Tfam was aberrantly modified, immunoprecipitation experiments against Tfam from both hepatic and renal tissues from MMA mice and controls were performed using a Tfam-specific antibody (Boster Biological Technology, PB9447). In both instances, increased malonylation/methylmalonylation on Tfam purified from MMA mice but not control mice was observed (FIGS. 12A-12B). Additionally observed was increased co-immunoprecipitation of mitochondrial DNA-directed RNA polymerase (Polrmt), an essential mtDNA transcription enzyme recruited to mtDNA promoters by Tfam where it forms a transcription complex with Tfam, and mitochondrial transcription factor B2 (Tfb2m), in MMA mouse hepatic extracts compared to controls (FIG. 12A). Tandem mass spectrometry analysis identified Polrmt as a protein with increased malonylation in MMA hepatic extracts compared to controls, and this result was confirmed via co-immunoprecipitation with Tfam and it was additionally discovered that Polrmt exhibits increased propionylation in MMA mice compared to controls (FIG. 12A). Increased acylation of these transcription factors, especially negatively charged PTMs such as malonylation and methylmalonylation could lead to reduced protein activity as both acylation and phosphorylation (a negatively charge PTM) have been implicated in altering Tfam function through altering DNA binding affinity and promotor localization. Reduced function of Tfam and Polrmt would lead to reduced mtDNA copy number as well as reduced transcription of mtDNA encoded enzymes such as those that compose portions of the electron transport chain. To examine this possibility, mtDNA copy number in both human and mouse MMA hepatic extracts was assessed compared to controls (FIGS. 12C-12D). Both human and mouse MMA tissue exhibited significantly reduced mtDNA copy number compared to their respective controls as determined by performing real-time PCR (RT-PCR) for L strand encoded ND6 and/or H strand encoded COXI further indicating aberrant MMA specific acylation of mitochondrial regulatory proteins could contribute to pathophysiology (FIGS. 12C-12D). However, loss of expression of electron transport chain complexes could also be attributed to loss of mitochondrial cristae folds.

Cristae folds are maintained by optic atrophy 1 (OPAL), a dynamin-related GTPase which also regulates mitochondrial fission and fusion. These functions are regulated by both short and long isoforms of OPA1, the long isoforms are integrated into the membrane of the mitochondrial matrix while short isoforms remain soluble. Both long and short isoforms have been implicated in cristae fold formation and hyperacetylation has been documented to reduce OPA1 function. Additionally, morphological changes in mitochondrial structure seen in MMA tissues (e.g., megamitochondria and loss of cristae) are highly similar to mitochondrial structural changes observed in fruit fly wings treated with Opal RNAi. Therefore, if OPA1 exhibited increased acylation in MMA, it could mimic the inhibitory effects of acetylation leading to loss of OPA1 function resulting in loss of cristae folds and potentially dysregulated mitochondrial fusion/fission. To examine this possibility, immunoprecipitation analysis of Opal from renal tissue extracts obtained from MMA and control mice was performed (FIG. 12E). Interestingly, long isoforms of Opal demonstrated increased propionylation while short isoforms exhibited increased malonylation/methylmalonylation in

MMA mice compared to controls (FIG. 12E). As demonstrated in prior examples, hyper-acylation can alter protein charge which disrupts function, however MMA associated hyper-acylation can also result in protein dysregulation by blocking the placement of other regulatory PTMs that also take place on lysine residues.

This data further demonstrates the use of targeted pathway analysis to further extend and complement proteomic discovery to identify targets for therapeutic intervention.

It was proposed that aberrant PTMs cannot be sufficiently removed by sirtuins (SIRTs), which leads to the disruption of target activity, and subsequent disease manifestations. Using the in vitro deacylation assay described above, candidate SIRTs were overexpressed, then purified and used to assess demethylmalonylation of pre-modified BSA. Briefly, methylmalonylated BSA substrate was incubated with individual purified SIRT proteins, +/−NAM, +/−NAD⁺, and the presence of PTMs analyzed using anti-malonyllysine antibody and Western blotting. Methylmalonylated BSA or propionylated BSA was prepared as described above with 2.5 mM methylmalonyl-CoA. FLAG-SIRT1 (Addgene, 1791), SIRT2-FLAG (Addgene, 102623), SIRT3-FLAG (Addgene, 13814), SIRT4-FLAG (Addgene, 13815), SIRT5-FLAG (Addgene, 13816; SEQ ID NO: 8), SIRT6-FLAG (Addgene, 13817), or SIRT7-FLAG (Addgene, 13818) was overexpressed in 293T cells using lipofectamine 2000 chemical transfection reagent and immunoprecipitated from cell lysates prepared using M-PER lysis reagent (Thermo Scientific, 78501) with protease inhibitor cocktail using anti-Flag antibody conjugated Beads (Sigma, EZview™ Red ANTI-FLAG® beads). Sirtuin enzyme was eluted from Flag beads by incubating sirtuin-flag bound beads with 3 μl of 3× FLAG peptide stock (5 mg/ml) per 500 ul of cell lysate (Sigma, FLAG® peptide F3290-4MG). Eluted FLAG-SIRT1, SIRT2-FLAG, SIRT3-FLAG, SIRT4-FLAG, SIRTS-FLAG, SIRT6-FLAG, or SIRT7-FLAG protein was quantified by Coomassie stain. Approximately 15 μg of modified BSA was incubated with or without 1 μg of FLAG-SIRT1, SIRT2-FLAG, SIRT3-FLAG, SIRT4-FLAG, SIRTS-FLAG, SIRT6-FLAG, or SIRT7-FLAG, with or without 0.1 mM NAD (sirtuin co-factor), and with or without sirtuin inhibitor nicotinamide (NAM) at 40 μM. Reactions were incubated on a thermomixer at 30° C. for 3 hours at 600 RPM. Reactions were stopped by boiling in SDS buffer and run on SDS page. Resulting westerns were stained with ponceau to find total BSA protein levels and then stained for methylmalonyl-lysine or propionyl-lysine and SIRT1 (Proteintech, 13161-1-AP), SIRT2 (Proteintech, 19655-1-AP), SIRT3 (Proteintech, 10099-1-AP), SIRT4 (GeneTex, GTX51798), SIRT5 (Proteintech, 15122-1-AP), SIRT6 (Novus, NB100-2522), or SIRT7 (Proteintech, 12994-1-AP).

As shown in FIGS. 12A and 12B, SIRT1 (FIG. 13A) and SIRT5 (FIG. 13B) were the most effective at removing methylmalonylation from the methylmalonylated BSA substrates (e.g., only SIRT1 and SIRT5 demonstrated the strongest deacylase activity against methylmalonylation).

### Example 2

### SIRT5 Mediates De-Propionylation Activity

Based on the observation in Example 1 that endogenous Sirtuins fail remove the hyperacylation from CPS1 in isolated MMA patient samples, it was investigated whether SIRT5 itself is inhibited via hyperacylation.

Propionylated BSA was prepared as described in Example 1 with 2.5 mM propionyl-CoA (Sigma, P5397-10mg). SIRT5-FLAG was purified and quantitated as described in in Example 1 and then incubated with or without 2.5 mM propionyl-CoA in Tris-Cl buffer (50 mM Tris-Cl and 150 mM NaCl at a pH of 8 at 37 C) at a concentration of 1 μg/μl on a thermomixer for 9 hours at 37° C. at 600 RPM. Modified and unmodified SIRT5-FLAG was concentrated using Micron®-10 centrifugal filters Ultracel® PL-10 units (Millipore). Approximately 15 ng of modified BSA was incubated with or without 1 ng of unmodified SIRT5-FLAG or modified SIRT5-FLAG, with or without 0.1 mM NAD (sirtuin co-factor), and with or without sirtuin inhibitor nicotinamide (NAM) at 40 μM. Reactions were incubated on a thermomixer at 30° C. for 3 hours at 600 RPM. Reactions were stopped by boiling in SDS buffer and run on SDS page.

Resulting westerns were stained with ponceau to find total BSA protein levels and then immunoblotted for propionyl-lysine and SIRT5.

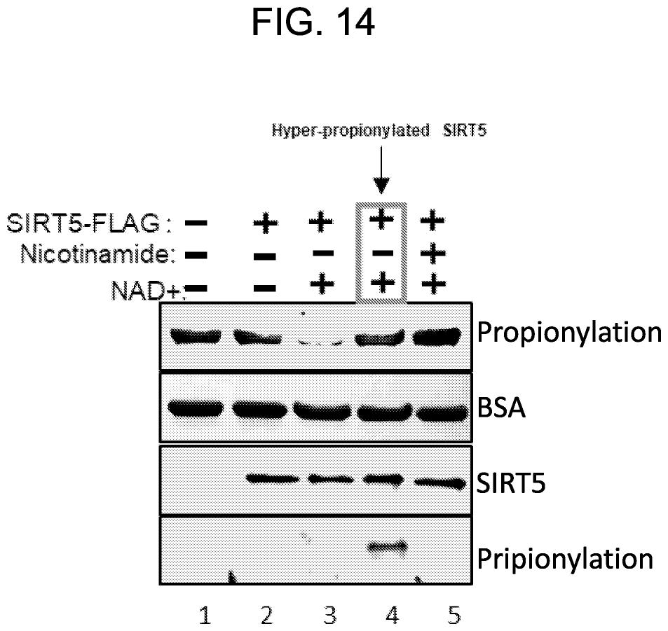

As shown in lane 3 of FIG. 14, SIRT5 mediates the depropionylation of BSA substrate. However, this activity is impaired when SIRT5 is propionylated (lane 4 of FIG. 14). These experiments demonstrate a novel enzymatic activity for SIRT5 originally only seen for SIRT1 (depropionylation), and indicate that hyper-acylation of SIRT5 results in lower enzymatic activity compared to unmodified SIRT5.

Based on these observations, it was concluded that in isolated MMA patients, SIRT5 is likely hyperacylated (hypermethylmalonylated, hyperpropionylated, and/or hypermalonylated) and thus inactivated, which prevents SIRT5 from deacylating and activating CPS1. That is, the PTMs on SIRT5 reduce or prevent native deacylase activity (e.g., deacylation of CPS1 and GCSH (Protein H)) in isolated

MMA patients resulting in hyperammonemia and hyperglycinemia.

### Example 3

### SIRT5 K4R Mutant Resistant to Inhibition after Hyper-Methylmalonylation/Acylation Modifications

In view of the data in Example 2 showing that the SIRTs themselves can be inactivated by hyperacylation, a SIRT5 was developed that is resistant to acylation inactivation. Critical lysine residues were changed to arginine, a deacylated lysine mimic

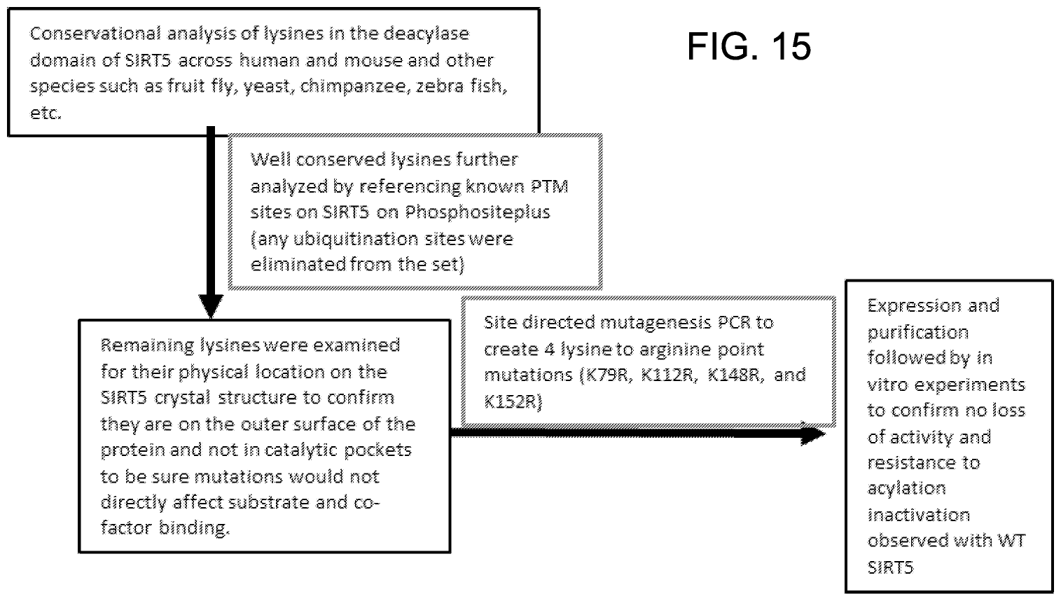

As shown in FIG. 15, using conservation analysis and considering the structure of SIRT5, four highly conserved external lysine residues in the deacylase domain were identified (K79, K112, K148, K152, see SEQ ID NO: 2). Briefly, human SIRT5 protein has 14 lysine residues that were tested for conservation via Blastp analysis of SIRT5 from human, chimpanzee, mouse, frog, chicken, zebra fish and fruit fly. Residues with low conservation or residues in the mitochondrial leader sequence or outside of the deacylase enzymatic domain were eliminated. PhosphoSitePlus® was used to eliminate lysine residues in SIRT5 involved in ubiquitination/protein turnover. The published 3D structure of human SIRT5 protein (RCSB 3RIY) showed that four of the remaining surface lysine residues around the catalytic SIRT5 deacylase domain were to be mutated to arginine (mimic of non-acylated lysine). Using sited directed mutagenesis and the following primers, K79, K112, K148, and K152 were mutated to arginine and validated by whole plasmid sequencing (plasmids and AAV sequences shown in SEQ ID NOS: 11 and 12).

K79R forward:
(SEQ ID NO: 13)
5′-GTTATTGGAGAAGATGGCAAGCC-3′
K79R reverse:
(SEQ ID NO: 14)
5′-GGCTTGCCATCTTCTCCAATAAC-3′
K112R forward:
(SEQ ID NO: 15)
5′-CATGGGGAGCAGGGAGCCCAACG-3′
K112R reverse:
(SEQ ID NO: 16)
5′-CGTTGGGCTCCCTGCTCCCCATG-3′
K148/152R forward:
(SEQ ID NO: 17)
5′-CTGCACCGCAGGGCTGGCACCAGGAACCTTCTG-3′
K148/152R reverse:
(SEQ ID NO: 18)
5′-CAGAAGGTTCCTGGTGCCAGCCCTGCGGTGCAG-3′

K79, K112, K148, and K152 were mutated to arginine by site-directed mutagenesis, and tested for enzymatic activity before and after hyper-methylmalonylation using the methods provided in Example 2. The resulting mutated SIRT5 has K79R, K112R, K148R, and K152R point mutations, and is referred to herein as K4R SIRT5 (SEQ ID NO: 4). These mutated residues cannot accept acyl groups.

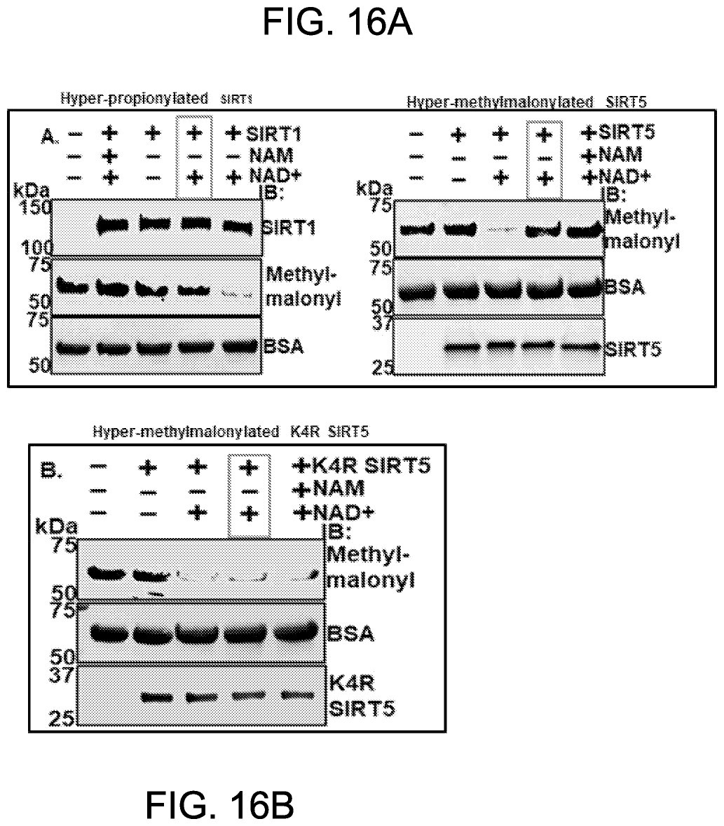

FLAG-SIRT1 or SIRT5-FLAG was modified with methylmalonylation and assayed for enzymatic activity in vitro as described Example 2. As shown in FIG. 16A, native SIRT5 can remove methylmalonylation from BSA (lane 3 of FIG. 16A, right panel), but is unable to do so when methylmalonylated (lane 4 in FIG. 16A, right panel). Similar results were observed for SIRT1 (FIG. 16A, left panel). K4R mutated SIRT5-FLAG was also purified and modified in the same fashion and its enzymatic activity tested on methylmalonyl-BSA substrate. In contrast, SIRT5 K4R did not exhibit enzymatic inhibition after hyper-methylmalonylation/acylation modifications (lane 4 of FIG. 16B) and exhibited resistance to inhibition by nicotinamide. That is, mutant SIRT5 K4R had the ability to remove methylmalonylation from BSA regardless of whether SIRT5 K4R was acylated or not.

### Example 4

### Deacylation Activity of SIRT5 K4R on Liver Extracts

The following method was used to determine the enzymatic activity of SIRT5 K4R (SEQ ID NO: 4) in vitro on non-BSA methylmalonylated/malonylated protein targets. Liver tissue extract from one Mmut^{-/-}; Tg^{INS-MCK-Mmut} mouse was split into three separate tubes (30 μg total protein per tube) and incubated without SIRT5 K4R, with 1 μg SIRT5 K4R and NAD+, and with 1 μg SIRT5 K4R, NAD+ and Nicotinamide (NAM). The in vitro deacylation reactions were performed at 37° C. shaking at 400 RPM for 6 hours before stopping the reaction by adding SDS loading buffer and boiling for 5 minutes. Reactions were run on SDS-Page gels alongside 30 ug of Mmut^{-/-}; Tg^{INS-MCK-Mmut} control sample. The resulting gels were blotted and stained for with anti-malonyllysine antibody, anti-proionyllysine antibody, f3-actin antibody, and SIRT5 antibody.

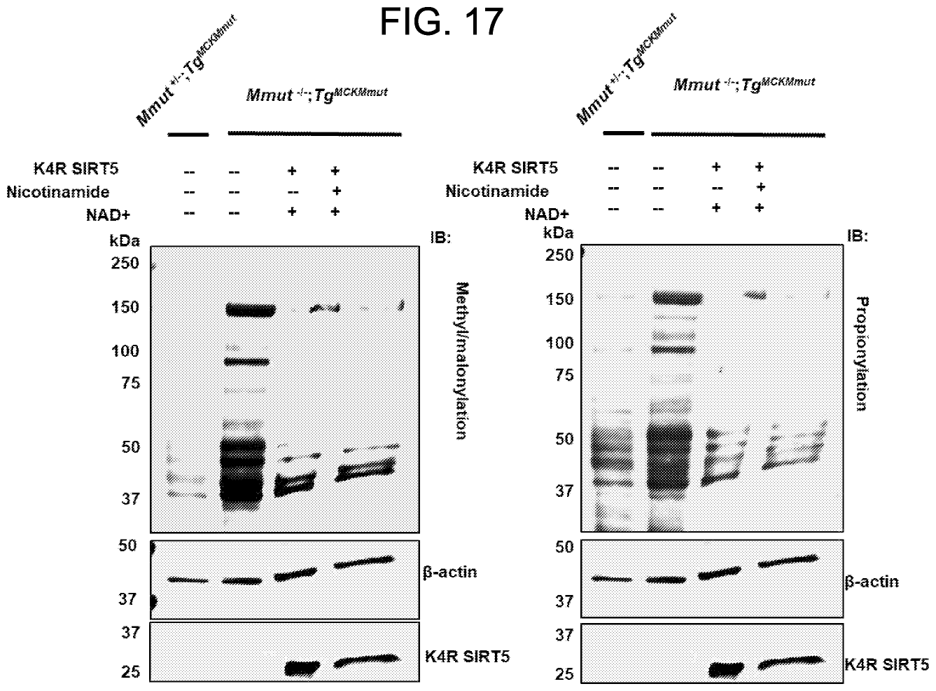

As shown in FIG. 17, the presence of the SIRT5 K4R mutant in liver extracts from MMA model mice demonstrated significantly less acylation (methyl/malonylation and propionylation, see lanes 4 and 8), as compared to such activity without the SIRT5 K4R mutant (lanes 2 and 6). These results demonstrate that SIRT5 mutants with lysine residues mutated to arginines that retain de-acylation activity when hyperacylated, can be used to deacylate proteins (such as CPS1 and GCSH) in subjects with an OA, such as MMA.

### Example 5

### Treatment of MMA Mice with Mutant SIRT5

This example describes methods for treating OA in Mmut^{-/-}; Tg^{INS-MCK-Mmut} mice, by expressing SIRT5 K4R in the mice from an AAV vector (e.g., SEQ ID NO: 11 or 12) and/or by administration of one or more SIRT5 activators.

### Methods

The following methods were used to examine the effect of SIRT5 K4R expression in MMA mice (Mmut^{-/-}; Tg^{INS-MCK-Mmut}).

### Immunoprecipitation:

Harvested liver tissue was homogenized using sterile homogenizer tubes and pestles on ice in T-PER™ (ThermoFisher Scientific) supplemented with fresh protease inhibitors (cOmplete™ Protease Inhibitor Cocktail, EDTA-free Sigma). Homogenates were centrifuged at 16,000 RCF for 10-15 minutes. Supernatant was collected and measured for protein content by Braford assay. Target proteins from 3 mg of lysate were captured with CPS1 (Abcam, ab129076) targeted antibody or IgG antibody (normal rabbit IgG EMD Millipore 12-370) conjugated to protein A agarose beads (Invitrogen) following pre-clear with unconjugated beads. Complexes were washed four times with IP washing buffer (T-PER™) supplemented with protease inhibitors before denaturation in 5x SDS loading buffer and SDS-PAGE analysis. Detection was performed with the Odyssey system using the following secondary antibodies: IRDye® 800CW Donkey anti-Rabbit IgG Secondary Antibody (LI-COR) and IRDye® 680RD Donkey anti-Mouse IgG (LI-COR). The resulting Western blots were stained with β-actin antibody (Proteintech, 66009-1-Ig), anti-CPS1 (Abcam, ab129076), anti-FLAG (Sigma, F3165-1MG), and anti-acyllysine antibody AP42053, a polyclonal antibody generated to detect methylmalonylation. From the proteome data gained from tandem mass spectrometry experiments, a subset of methylmalonylated peptides were selected to generate a motif for polyclonal methylmalonylation antibody production in rabbits. The sequences used were as follows: KKAKNKQLGHEEDYALGKD (SEQ ID NO: 42), KKKEKEVKK (SEQ ID NO: 43), KTAHIVLEDGTKMKG (SEQ ID NO: 44), KISLPHPMEIGENLDGTLKSRKRRK (SEQ ID NO: 45), KKKNDFEQGELYLKE (SEQ ID NO: 46), KDKYKQIFLGGVDKR (SEQ ID NO: 47),

KGKKLVKKKIGKKDAGKKEGKC (SEQ ID NO: 48), KKNSEGLLKNKEKNQKL (SEQ ID NO: 49), KDAYIKKQNLEKA (SEQ ID NO: 50), KAFKNKETLIIEPEKN (SEQ ID NO: 51), KDVEKKLNKVTKF (SEQ ID NO: 52), KELGEKISQLKDELKT (SEQ ID NO: 53), KKIVAENHLKKI (SEQ ID NO: 54), RKKVETEAKIKQKL (SEQ ID NO: 55), KKETKGPAAENLEAKPVQAPTVKKAEKD (SEQ ID NO: 56), KKFGGQDIFMTEEQKKYYNAMKKL (SEQ ID NO: 57), KKDTQTKSIISETSNKIDTEIASLKTLMESSKL (SEQ ID NO: 58), KLGKMDRVVLGWTAVFWLTAMVEGLQVTVPDKKK (SEQ ID NO: 59), KYKIKTIQDLVSLKE (SEQ ID NO: 60), and KRKMRKGQHLDLKA (SEQ ID NO: 61). Resulting polyclonal antibodies were tested against acylated BSA substrates and human liver tissue extracts from control and MMA patient samples to determine which antibody had an optimal reactivity profile for further experimental use. Detection was performed with the Odyssey imaging system using the following secondary antibodies: IRDye® 800CW Donkey anti-Rabbit IgG Secondary Antibody (LI-COR) and IRDye® 680RD Donkey anti-Mouse IgG (LI-COR).

### Ammonia Assay:

The ammonia assays were performed using the ammonia assay kit from Millipore Sigma (AA0100-1KT). Serum from terminal mouse retro-orbital bleeds were filtered on Microcon-10kDa Centrifugal Filter Units with Ultracel-10 membrane from Millipore Sigma (MRCPRT010) to deproteinate followed by ammonia level measurement using the ammonia assay kit provided instructions.

### Immunoblot:

Harvested liver tissue was homogenized using sterile homogenizer tubes and pestles on ice in T-PER™ (ThermoFisher Scientific) supplemented with fresh protease inhibitors (cOmplete™ Protease Inhibitor Cocktail, EDTA-free Sigma). Homogenates were centrifuged at 16,000 RCF for 10-15 minutes. Supernatant was collected and measured for protein content by Braford assay. Samples were then denatured in 5× SDS loading buffer and run on SDS-PAGE gels. Resulting blots were immunoblotted with one or more of the indicated antibodies: β-actin antibody (Proteintech, 66009-1-Ig), anti-propionyl antibody (PTM Biolabs, PTM-203), anti-SIRT5 (Proteintech, 15122-1-AP), anti-methylmalonyl antibody (AP42053), anti-CPS1 (Abcam, ab129076), anti-GCSH (Proteintech, 16726-I-AP), anti-lipoic acid (Abcam, ab58724), and anti-FLAG (Sigma, F3165-1MG). Detection was performed with the Odyssey imaging system using the following secondary antibodies: IRDye® 800CW Donkey anti-Rabbit IgG Secondary Antibody (LI-COR) and IRDye® 680RD Donkey anti-Mouse IgG (LI-COR).

### Results

Six Mmut^{-/-}; Tg^{INS-MCK-Mmut} and three Mmut^{-/-}; Tg^{INS-MCK-Mut} mice were treated with 1×10¹³ GC/kg of an AAV8 vector comprised of a transgene that uses the TBG promoter to drive the expression of SIRTS K4R (SEQ ID NO: 11) in the liver, abbreviated AAV8 TBG SIRT5 K4R. FiveMmut^{-/-}; Tg^{INS-MCK-Mmut} and three Mmut^{-/-}; Tg^{INS-MCK-Mmut} were treated with 1×10¹³ GC/_{kg} of an AAV8 TBG SIRT5 K4R or an AAV8 TBG GFP control (FIG. 18A). Mice were between 2 and 3 months of age and were weighed before treatment and then twice a week every week following treatment. As shown in FIG. 18B, after 2 months of treatment, the SIRTS K4R-treated Mmut^{-/-}; Tg^{INS-MCK-Mmut} exhibited a significant increase in percent body weight compared to the GFP-treated Mmut^{-/-}; Tg^{INS-MCK-Mmut} control mice as determined by student t-test (* P-value<0.05).

In addition, organ tissues can be harvested to examine SIRTS K4R expression, PTMs, and other markers of metabolic homeostasis. Further analysis revealed that the AAV8 TBG SIRTS K4R compared to AAV8 TBG GFP control treated mice had robust SIRTS K4R expression (FIG. 18A), increased body weight in the case of AAV8 TBG SIRTS K4R-treated Mmut-/-;TgMCKMmut mice (FIG. 18B), and diminished methylmalonylation in hepatic extracts (FIG. 18C). Propionylation was relatively unaffected (FIG. 18D).

A series of further studies to examine specific targets of methylmalonylation, specifically directed toward Cpsl, were also performed. As shown in FIG. 18E (top), all groups of mice expressed Cpsl, however immunoprecipitation with subsequent probing with an anti-methylmalonyllysine antibody showed that only the AAV8 TBG SIRTS K4-treated Mmut-/-;TgMCKMmut mice, but not controls or those treated with an AAV that encoded GFP, had removal of the aberrant acylation of Cpsl (FIG. 18E, bottom). Theses AAV8 TBG SIRTS K4R-treated Mmut-/-;TgMCKMmut mice had lower serum ammonia (FIG. 18F). Furthermore, SIRTS K4R treatment rescued lipoylation of H-protein in the livers of Mmut^{-/-}; Tg^{INS-MCK-Mmut} compared to GFP-treated controls (FIG. 18G), further demonstrating that Sirtuin-based therapies can rescue secondary effects of MMA pathophysiology.

One skilled in the art will appreciate that other AAV vectors encoding SIRTS K4R can be used, such as SEQ ID NO: 12, where the promoter is the enhanced chicken beta actin.

### Example 6

### Kits to measure SIRT5 Activity toward Acyl-PTMs

This example describes methods and kits that can be used to measure SIRT5 activity toward the removal of methylmalonylation and other acyl-PTMs. Using the methods and techniques presented herein, purified SIRT5 or a SIRT5 mutant provided herein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions) can be used with acylated BSA, such as methylmalonylBSA. For example, purified SIRTS or SIRTS K4R can be added to or contacted with a sample that is suspected to be acylated, and the metabolism of said sample monitored in parallel with a control containing the acylated BSA (e.g., methylmalonylated BSA) and SIRTS or a SIRTS mutant provided herein. Exemplary samples include an organ (e.g., liver) or cellular extract, such as an extract obtained after or with treatment or with co-incubation, of small molecule drugs, toxins, nucleic acids, or prodrugs. The control in the kit (e.g,. methylmalonylated BSA) or an affected hepatic extract (FIG. 17) is processed in an identical fashion and compared to the test sample to measure metabolism by, or inhibition of, SIRTS or a SIRTS mutant provided herein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions). Thus, in some examples, the kit includes purified SIRTS, purified SIRTS mutant provided herein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions) (or both WT and mutant SIRTS), acylated BSA (such as methylmalonylated BSA), and optionally an extract, such as a liver extract from a normal or MMA subject. Such components of the kit can be in separate containers, such as glass or plastic vials.

In another example, the conditions are related to a nutritional or absorptive defect of vitamin B12, or related to a metabolic disorder where the metabolism of vitamin B12 is impaired, such as deficiency of MMACHC (cb1C), MMADHC (cb1D), or LMBDR1 (cb1F). In another embodiment, the sample is an extract and the assay is coupled to the release 2′-O-Methylmalonyl-ADP-ribose and 3′-O-Methylmalonyl-ADP-ribose (OMMADPr) or related 2′-O-Acyl-ADP-ribose and 3′-O-Acyl-ADP-ribose (OAADPr) by the action of SIRTS or a SIRTS mutant provided herein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions), and measured in a high throughput fashion to identify activators or inhibitors of SIRTS or mutant SIRTS enzyme activity. Compounds identified can be new drugs that mitigate the untoward effects of organic acid metabolism (i.e., activators), or potentiate it (i.e., inhibitors).

In yet another embodiment, the sample is a cell line and the SIRTS or a SIRTS mutant provided herein are expressed as nucleic acids, and the cell line is then subjected to a screen to measure the release of 2′-O-Methylmalonyl-ADP-ribose and 3′-O-Methylmalonyl-ADP-ribose (OMMADPr) or related 2′-O-Acyl-ADP-ribose and 3′-O-Acyl-ADP-ribose (OAADPr) by the action of SIRTS or a SIRTS mutant provided herein, and measured in a high throughput fashion to identify activators and inhibitors of SIRT5 or a SIRT5 mutant provided herein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions) enzyme activity. Compounds identified could be new drugs that mitigate the untoward effects of organic acid metabolism (i.e., activators), or potentiate it (i.e., inhibitors).

In yet another embodiment, the sample is a cell line and the SIRT5 or a SIRT5 mutant provided herein (such as a protein having at least 90%, at least 91%, at least 92%, at least 93%, at least 94%, at least 95%, at least 96%, at least 97%, at least 98%, at least 99%, or 100% sequence identity to SEQ ID NO: 4, wherein the protein retains an arginine at one or more of positions 79, 112, 148, and 152 of SEQ ID NO: 4, such as at all four positions) are expressed as nucleic acids, and the cell line is then subjected to a screen to measure the release of 2′-O-Methylmalonyl-ADP-ribose and 3′-O-Methylmalonyl-ADP-ribose (OMMADPr) or related 2′-O-Acyl-ADP-ribose and 3′-O-Acyl-ADP-ribose (OAADPr) by the action of other nucleic acids delivered as siRNAs, transgenes, or genome editing agents (Cas/CRISPR). siRNAs, transgenes, or genome editing agents and their corresponding encoded genes or targets could encode and identify new genes that encode activities including those that promote the untoward effects of organic acid metabolism (i.e., activators), or potentiate it (i.e., inhibitors). The corresponding genes, mRNAs, and their encoded enzymes represent new targets.

In view of the many possible embodiments to which the principles of the disclosure may be applied, it should be recognized that the illustrated embodiments are only examples of the disclosure and should not be taken as limiting the scope of the invention. Rather, the scope of the invention is defined by the following claims. We therefore claim as our invention all that comes within the scope and spirit of these claims.

1. An isolated mutant sirtuin 5 (SIRTS) protein comprising at least 80%, at least 81%, at least 82%, at least 83%, at least 84%,at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 4 or SEQ ID NO: 10, wherein the protein retains an arginine at one or more of positions 79, 112, 148, and 152 of SEQ ID NO: 4 or SEQ ID NO: 10.

2. The isolated mutant SIRTS protein of claim 1, wherein the protein retains an arginine at all of positions 79, 112, 148, and 152 of SEQ ID NO: 4 or SEQ ID NO: 10.

3. The isolated mutant SIRTS protein of claim 1, wherein the protein comprises or consists of the protein sequence of SEQ ID NO: 4 or SEQ ID NO: 10.

4. The isolated mutant SIRTS protein of claim 1, wherein the protein further comprises a purification tag.

5. An isolated nucleic acid molecule encoding the isolated mutant SIRTS protein of any one of claim 1.

6. The isolated nucleic acid molecule of claim 5, wherein the isolated nucleic acid molecule comprises at least 80%, at least 81%, at least 82%, at least 83%, at least 84%,at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3 or SEQ ID NO: 9, and encodes an arginine at one or more of positions 79, 112, 148, and 152 of SEQ ID NO: 4 or SEQ ID NO: 10.

7. The isolated nucleic acid molecule of claim 5, wherein the isolated nucleic acid molecule comprises at least 80%, at least 81%, at least 82%, at least 83%, at least 84%,at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3 or SEQ ID NO: 9, and encodes an arginine at all of positions 79, 112, 148, and 152 of SEQ ID NO: 4 or SEQ ID NO:

10.

8. The isolated nucleic acid molecule of claim 5, further comprising a promoter operably linked to the isolated nucleic acid molecule encoding the protein.

9. A vector comprising the isolated nucleic acid molecule of claim 5.

10. The vector of claim 9, comprising at least 80%, at least 81%, at least 82%, at least 83%, at least 84%,at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 11 or SEQ ID NO: 12 and encoding an arginine at 1, 2, 3, or 4 of positions 79, 112, 148, and 152 of SEQ ID NO: 4 or SEQ ID NO: 10.

11. The vector of claim 9, wherein the vector is a plasmid vector or a viral vector.

12. The vector of claim 11, wherein the viral vector is adeno-associated virus.

13. A host cell comprising the vector of claim 9.

14. The host cell of claim 13, wherein the host cell is a bacterium, human, or yeast cell.

15. A composition, comprising:
the isolated protein of claim 1, or a nucleic acid molecule encoding the isolated protein; and
a pharmaceutically acceptable carrier.

16. The composition of claim 15, further comprising:
one or more of L-carnitine, hydroxycobalamin, vitamin B12, an antibiotic, sodium benzoate, N-carbamylglutamate or combinations thereof; and/or
one or more of a MMUT, MMAA, MMAB, MMACHC, MMACHD, LMBRD1, MCEE, PCCA or PCCB protein or nucleic acid molecule encoding a MMUT, MMAA, MMAB, MMACHC, MMACHD, LMBRD1, MCEE, PCCA or PCCB protein.

17-18. (cancelled)

19. A method of treating an organic acidemia (OA), vitamin B12 deficiency, or metabolic disorder, comprising:
administering a therapeutically effective amount of the nucleic acid molecule of claim 5 to a subject having an OA, vitamin B12 deficiency, or metabolic disorder, thereby treating the OA, vitamin B12 deficiency, or metabolic disorder.

20. A method of treating an organic acidemia (OA), vitamin B12 deficiency, or metabolic disorder, comprising:
detecting one or more proteins posttranslationally modified with methylmalonyllation in a sample from a subject having or suspected of having an OA, vitamin B12 deficiency, or metabolic disorder; and
administering a therapeutically effective amount of the nucleic acid molecule of claim 5 to the subject having or suspected of having the OA, vitamin B12 deficiency, or metabolic disorder, thereby treating the OA, vitamin B12 deficiency, or metabolic disorder.

21. (canceled)

22. A method of reducing post-translational modifications (PTMs) of proteins in a subject having an OA, vitamin B12 deficiency, or metabolic disorder, or a method of reducing blood glycine and/or urine glycine by at least 10% in a subject having an OA, or a method of reducing blood ammonia by at least 10% in a subject having an OA, comprising:
administering a therapeutically effective amount the nucleic acid molecule of claim 5 to the subject having the OA, vitamin B12 deficiency, or metabolic disorder, thereby reducing PTMs of proteins in the subject having OA, a vitamin B12 deficiency, or metabolic disorder, or reducing blood and/or urine glycine by at least 10% in the subject having OA, or reducing blood ammonia by at least 10% in the subject having OA.

23-24. (canceled)

25. The method of any one of claims 20, wherein the OA is methylmalonic acidemia (MMA), isovaleric acidemia (IVA), glutaric acidemia type 1 (GA1), or propionic acidemia (PA).

26. The method of claim 20, wherein the one or more proteins are selected from the group consisting of carbamoyl phosphate synthetase (CPS1), glycine cleavage system H protein (GCSH), SIRT1, SIRT3, SIRT4, SIRT5, mitochondrial transcription factor A (TFAM), and optic atrophy 1 (OPA1).

27. The method of claim 20, wherein the one or more proteins are selected from the group consisting of: Cpsl, Aass, Atxn2, Cttn, F8, Hmgcl, Lrrn3, Nepro, Plin4, Rbm15, Tmem143, Argl, Cct5, Dip2b, Fat2, Harsl, Klrblf, No18, Ptprv, Slclal, Tkfc, Gstm7, Acaa2, Bclaf3, Cwc27, Fam184b, Hmgcs2, Mapls, Nipbl, Plxndl, Rbm27, Topors, Asap3, Cgn, Dnahl, Fgf8, Haus7, Lactb, Nsd3, Rasgeflb, Slc25a1, Tpp2, Hars, Acad8, Bdpl, Cyfip2, Fgr, Hnrnpc, Map2k6, Nipsnapl, Polq, Rev31, Trdn, Adhl, Asl, Chat, Dnah5, Fkbp5, Hibadh, Lyar, PHF20, Rdx, Slc25a5, Tsks, Mdgal, Acinl, C9, Depdc5, Fmrl, Hp, Mapkl, Nodl, Ppplr10, Rida, Trim21, Adnp, Assl, Cisdl, Dockl, Foxc2, Hmcnl, Macfl, Palm, Recq15, Slit3, Ttbk2, Rp1, Aco2, Ccdc40, Dhrsl, Gca, Hpfl, Mb12, Nono, Ppplrl2a, Rprdla, Aebpl, Atp5f1b, Clipl, Dock8, Gapdh, Hs3st3a1, Mcurl, Pcnx3, Rgs3, Sodl, Ttc28, Acsf2, Ccdc90b, Dhx9, Gldn, Hydin, Mctpl, Nudt13, Prcp, Rrsl, Ttn, Aifml, Atp5po, Cmya5, Dock9, Gcc2, Hsf3, Mett117, Pcskl, Sosl, Ushbpl, Adgrbl, Ccdc91, Dip2a, Gludl, Idh2, Mdh2, Nup50, Prdx5, Scnla, Tut7, Akap12, Atr, Co120a1, Dpp6, Gcic, Ids, Mett13, Pdelb, Rmdn3, Sptanl, Vdac3, Adsl, Cdk15, Dlst, Glyat, Igfn1, Mix23, Obscn, Prkcsh, Usp36, Akr1c6, Atxn713, Co124a1, Dym, Ift81, Mgstl, Pde4dip, Robol, Stab2, Vps13b, Agxt, Cep170, Dmgdh, Got2, Il4i1, Mmell, Optn, Prkdc, Slc7a3, Yeats2, Aldhla3, Bhlhe41, Eeal, Gpd2, Il10rb, Morc3, Pdia3, Rrbpl, Stk36, Vps25, Ankefl, Chmplbl, Dnajc14, Grk2, Inhba, Mmp13, Pask, Prr5, Smcla, Znf106, Aldh111, Blnk, Crisp2, Eeflal, Gpxl, Ildr2, Ms13, Pdzkl, Rtcb, Svil, Zbtb49, Asx11, Cit, Dst, Gtf2e1, Inpp5e, Mmrnl, Pc, Psmb2, Smc4, Znf770, Aldob, Bpifb6, Ctnna3, Efhb, Gstal, Isyl, Mug2, Pgkl, Rubcnl, Tbx2, Zc3h3, Atg14, Claspl, Dyncllil, Hadh, Kiaa1109, Mn1, Pclo, Ptchd4, Snrk, Acatl, Almsl, Bsdcl, Cyp2c37, Elp4, Gstml, Itsn2, Myhl, Pletl, Sec31a, Tcf20, Zfp28, Atmin, Coll1a2, Echl, Hadha, Lcp2, Mycbp, Pdia2, Rabepl, Sod2, Cs, Ankrd23, Byes, Cyp2u1, Em16, Gstpl, Kcnk2, Ndufafl, Sec63, Tedc2, Znf518a, Atp5pb, Col4a1, Ecil, Hba, Lefl, Mycbp2, Piddl, Raetlb, Tbrgl, Ccdc58, Ankrd34b, C1q13, Cyp3all, Eri2, Gstzl, Kdm2b, Nebl, Polr2h, Sez6, Tent2, Cyp2c50, Eppkl, Hibch, Lgr4, Naip5, Pla2g4c, Rapgef5, Tent4b, Certl, Anxa6, Ca3, Dbi, Fabpl, Gtpbpl, Kiflc, Nemf, Polrmt, Shc2, Tfap2a, Gsta3, Atp8b5, Ctdspl, Etfa, Hivepl, Lnpk, Nav3, Plaa, Rbbp6, Tlx2, Apexl, Didol, Fam189a1, Hadhb, Kif5b, Nfrkb, Pter, Skt, Tgfbr3, and Gstm2.

28. The method of claim 20, where the detecting comprises contacting the sample with an anti-methylmalonyllysine specific antibody or detecting the one or more proteins using mass spectrometry.

29. The method of claim 20, wherein the sample is a blood sample, plasma sample, urine sample, or liver biopsy sample.

30-31. (canceled)

32. The method of claim 20, further comprising administering a therapeutically effective amount of:
a MMUT, MMAA, MMAB, MMACHC, MMACHD, LMBRD1, or MCEE enzyme, a nucleic acid encoding the enzyme, or a vector encoding the enzyme;
a low-protein high calorie diet;
a diet that avoids isoleucine, valine, threonine, and methionine;
L-carnitine;
hydroxycobalamin;
vitamin B12;
one or more antibiotics;
sodium benzoate;
N-carbamylglutamate; or
combinations thereof.

33. The method of claim 20, wherein:
the metabolic disorder is methylmalonic acidemia, propionic acidemia, isovaleric acidemia, glutaric acidemia type 1, glutaric acidemia type 2, or methylglutaconic acidemia.

34. A kit, comprising:
an isolated native SIRT5 protein or nucleic acid molecule encoding an isolated native SIRT5 protein;
the isolated mutant SIRT5 protein of claim 1, or a nucleic acid molecule encoding the mutant SIRT5 protein;
nicotinamide (NAM);
nicotinamide adenine dinucleotide (NAD+);
an acylated protein; and/or an anti-acyllysine antibody.

35. The kit of claim 34, wherein:
the acylated protein is acylated bovine serum albumin (BSA); and/or
the anti-acyllysine antibody is specific for malonyllysine and methylmalonyllysine.

36-37. (canceled)

38. The kit of claim 34, further comprising:
a non-acylated version of the acylated protein;
a liver extract from a mammal with an OA, a liver extract from a normal mammal not having an OA, or both; and/or
a tissue, blood or urine sample from a subject with vitamin B12 deficiency.

39. (canceled)
